(19) United States
(12) Patent Application Publication
(10) Pub. No.: US 2024/0050512 A1
(43) Pub. Date: Feb. 15, 2024

(54) REDUCTION OF VIRAL INFECTIONS

(71) Applicant: PHOXBIO LIMITED, Dublin (IE)

(72) Inventors: Mohammed Abid Masood, London (GB); Rakesh Uppal, London (GB); Angela Russell, London (GB); Laurence Silpa, London (GB)

(21) Appl. No.: 18/265,981

(22) PCT Filed: Nov. 25, 2021
(86) PCT No.: PCT/EP2021/083029

§ 371 Date: Jun. 8, 2023

(30) Foreign Application Priority Data
Nov. 25, 2020 (GB) 2018571.6
Dec. 21, 2020 (GB) 2020320.4
Apr. 18, 2021 (GB) 2105509.0
Jul. 2, 2021 (WO) PCT/EP2021/068425

(51) Int. Cl. A61K 36/9068; A61K 36/61; A61K 36/53; A61K 36/88; A61K 36/185; A61K 36/71; A61K 38/40; A61K 33/30; A61K 33/26; A61K 9/08; A61K 45/06; A61P 31/14

(52) U.S. Cl. CPC A61K 36/9068; A61K 36/61; A61K 36/53; A61K 36/88; A61K 36/185; A61K 36/71; A61K 38/40; A61K 33/30; A61K 33/26; A61K 9/08; A61K 45/06; A61P 31/14

(57) ABSTRACT

The present invention relates to a composition comprising one or more of clove oil, eucalyptus oil, basil oil, ginger oil, an extract or component of any of these, or a combination thereof, and to a buffer composition having a pH of from 6.7 to 7.9 at a temperature of 37° C., both for use in the treatment, prophylactic treatment or amelioration of an airborne viral infection, or for use in reducing viral replication in a subject infected with an airborne virus or exposed to an airborne virus capable of causing an airborne viral infection in the subject. The present invention also relates to an aqueous composition either comprising a buffer and an essential oil with antiviral, antibacterial or antifungal activity; or comprising a buffer, hydrogen carbonate ions, and zinc ions; or comprising a buffer, hydrogen carbonate ions, and transferrin and/or iron ions. The present invention also relates to methods of preparing the compositions, and to concentrates of the compositions. The compositions can be used in a method of treatment, prophylactic treatment or amelioration of an airborne viral infection in a subject, and a method of reducing viral replication in a subject infected with an airborne virus or exposed to an airborne virus capable of causing an airborne viral infection in the subject. Airborne viruses include RNA viruses, such as coronaviruses, such as MERS-CoV, SARS-CoV, and SARS-COV-2. The compositions can be administered by nasal spray, inhaler or nebulizer, or in the form of a cream, gel or emulsion, and the invention therefore also relates to nasal sprays, inhalers, nebulizers, creams, gels or emulsions comprising the compositions. The compositions can also be applied to a mask or other face covering thereby reducing the risk of viral infection with an airborne virus, and the invention therefore also relates to sprays comprising the compositions. The compositions can also be used in a receptacle through which an oxygen-containing gas is bubbled prior to inhalation by a subject, and the invention therefore also relates to receptacles containing the compositions.

### FIELD OF THE INVENTION

The present invention relates to a composition comprising one or more of clove oil, eucalyptus oil, basil oil, ginger oil, an extract or component of any of these, or a combination thereof, and to a buffer composition having a pH of from 6.7 to 7.9 at a temperature of 37° C., both for use in the treatment, prophylactic treatment or amelioration of an airborne viral infection, or for use in reducing viral replication in a subject infected with an airborne virus or exposed to an airborne virus capable of causing an airborne viral infection in the subject. The present invention also relates to an aqueous composition either comprising a buffer and an essential oil with antiviral, antibacterial or antifungal activity; or comprising a buffer, hydrogen carbonate ions, and zinc ions; or comprising a buffer, hydrogen carbonate ions, and transferrin and/or iron ions. The present invention also relates to methods of preparing the compositions, and to concentrates of the compositions.

The compositions can be used in a method of treatment, prophylactic treatment or amelioration of an airborne viral infection in a subject, and a method of reducing viral replication in a subject infected with an airborne virus or exposed to an airborne virus capable of causing an airborne viral infection in the subject. Airborne viruses include RNA viruses, such as coronaviruses, such as MERS-CoV, SARS-CoV, and SARS-CoV-2.

The compositions can be administered by nasal spray, inhaler or nebulizer, or in the form of a cream, gel or emulsion, and the invention therefore also relates to nasal sprays, inhalers, nebulizers, creams, gels or emulsions comprising the compositions. The compositions can also be applied to a mask or other face covering thereby reducing the risk of viral infection with an airborne virus, and the invention therefore also relates to sprays comprising the compositions. The compositions can also be used in a receptacle through which an oxygen-containing gas is bubbled prior to inhalation by a subject, and the invention therefore also relates to receptacles containing the compositions.

### BACKGROUND OF THE INVENTION

All viruses infect and reproduce in a living host by entering the host body via a liquid carrier such as body fluids, sneezing fluids, or water droplets. Airborne viruses are those in which the disease spreads in particles in exhaled air. These particles include aerosols, which are smaller than 5 micrometres in diameter and which can remain airborne for extended periods, and also larger droplets through which transmission can occur over relatively short distances. Airborne viruses include (i) RNA viruses, such as coronaviruses (such as MERS-CoV, SARS-CoV, and SARS-CoV-2), influenza viruses (such as influenza A virus, influenza B virus, influenza C virus, and parainfluenza virus), rhinoviruses, Measles virus, Mumps virus, Rubella virus, and human respiratory syncytial virus, and (ii) DNA viruses, such as Parvovirus B19, adenoviruses and adeno-associated viruses, herpes viruses (such as Varicella-Zoster virus (VZV or HHV-3), Epstein-Barr virus (EBV or HHV-4), human herpes virus 6 (HHV-6A and HHV-6B), and human herpes virus 7 (HHV-7)), polyomaviruses (such as BK polyomavirus and WU polyomavirus), and Variola virus.

Airborne viruses enter the body via mucosal surfaces, and in particular, through the nasal and other airway epithelial surfaces. The nasal and other airway epithelia tend to be acidic, and can become more acidic, for example, due to gastroesophageal reflux.

The structural characteristics of virus coats (capsids) are highly relevant to virus propagation, because the coat must contain and protect the nucleic acid contents, be capable of broaching the outer membrane of a target cell, and provide a secure pathway for conducting nucleic acid into the target cell. Spike proteins on the capsid fulfil the latter two roles, mediating binding to cellular receptors, and the subsequent fusion of viral and cellular membranes. A motif comprising two heptad repeat regions and a hydrophobic fusion peptide (Dutch et al, Biosci Rep, 2000, vol 20(6), pages 597-612) is present in the spike proteins of SARS-CoV (Hakansson-McReynolds et al, J Biol Chem, 2006, vol 281(17), pages 11965-11971) and other coronaviruses (Xu et al, J Biol Chem, 2004, vol 279(47), pages 49414-49419), as well as the hemagglutinin (HA) of influenza (Skehel et al, Annu Rev Biochem, 2000, vol 69, pages 531-569) and other viruses.

Viral infection is initiated upon binding of the viral spike protein with a specific receptor or co-receptor on the target cell surface, mediated by the spike protein's receptor-binding domain (RBD). Thus, the RBD plays an important role in viral attachment, fusion and entry. Proteases found in the nasal and other airway epithelia coupled with an acidic environment cause the spike protein to hydrolyse and the spike protein undergoes a conformation change such that binding to the host cell membrane is favoured.

In influenza virus, the hemagglutinin (HA) spike protein is a homotrimeric glycoprotein which binds to the human mucosal epithelia followed by fusion of the two moieties. Cleavage activation of the HA precursor protein by protease enzymes is an essential step in the influenza virus replication cycle. HA cleavage activation is required for virus-endosome membrane fusion and the subsequent release of the influenza virus genome into the cytoplasm. Previous studies have shown that HA cleavage is most likely driven by either membrane-bound or extracellular trypsin-like proteases that reside in the respiratory tract (Hamilton, J Virol, 2012, vol 86(19), pages 10579-10586).

The new strain of coronavirus, SARS-CoV-2, the causative agent of the COVID-19 pandemic, was first identified in 2019 in Wuhan, China. The spike proteins of coronaviruses are different to those of the influenza HA protein; however, the overall mechanism of viral cellular attachment is similar and also requires an acidic environment. Both coronaviruses and influenza viruses require the acidic environment of the endosome to trigger their fusion with the cell membrane and delivery of the viral nucleocapsid into the cellular cytoplasm. It has recently been shown that SARS-CoV-2 utilizes host receptors, specifically angiotensin converting enzyme 2 (ACE2), and host proteases, for cell surface binding, priming and internalization. ACE2 is a member of the M2 family of metallopeptidases and contains a HEXXH motif that functions as the 25 zinc binding domain at its active site (Speth et al, The Faseb Journal, 2014, https://doi.org/10.1096/fasebj.28.1_supplement.1067.4). The structure of the extracellular portion of the SARS-CoV-2 spike protein in its prefusion conformation was recently published (Wrapp et al, Science, 2020, vol 367, pages 1260-1263), and the spike was shown to form trimers that bind ACE2 on host cells. Upon ACE2 engagement, within the acidic environment of the endosome, the spike changes from a prefusion to a post-fusion conformation (priming) as part of the cell entry process. Thus, coronaviruses use their spike proteins to select and enter target cells; SARS-CoV-2 uses the ACE2 receptor for entry and the cellular protease TMPRSS2 for priming.

The envelope of the SARS-CoV virus also comprises prominent projections formed by the spike protein, S (Li et al, J Virol, 2006, vol 80(14), pages 6794-6800). As with SARS-CoV-2, the SARS-CoV spike protein directs cell entry by binding to the receptor ACE2 on the cell surface (Li et al, Nature, 2003, vol 426, pages 450-454) and by fusing the viral and host membranes (Dave Cavanagh, The coronavirus surface glycoprotein, 1995, Plenum Press, New York). Several groups (Simmons et al, Antiviral Research, 2013, vol 100(3), pages 605-614) have identified cathepsins and type II transmembrane serine proteases as cellular activators of SARS-CoV and demonstrated that certain emerging viruses might trigger activation of the spike protein via these enzymes (Laporte et al, Current Opinion in Virology, 2017, vol 24, pages 16-24) to promote their spread, and this is dependent on epithelial pH. Yang et al (J Virol, 2004, vol 78(11), pages 5642-5650) reported that cellular entry of SARS-CoV is mediated by the spike glycoprotein and enhanced by dendritic cell transfer through pH-dependent endocytosis. Wang et al (Cell Research, 2008, vol 18, pages 290-301) reported that the entry of SARS-CoV into host cells was via a clathrin- and caveolae-independent pathway. Yang et al reported that the entry pathway was pH-dependent (Yang et al, J Virol, 2004, vol 78(11), pages 5642-5650) and required an acidic environment in the host cell interstitial fluid.

A protein encoded by SARS-CoV designated as open reading frame-9b (ORF-9b) localizes to the host cell mitochondria and causes mitochondrial elongation by triggering ubiquitination and proteasomal degradation of dynamin-like protein 1, a host protein involved in mitochondrial fission (Shi et al, J Immunol, 2014, vol 193(6), pages 3080-3089). The mitochondria is thus induced to increase ATP production to provide energy for viral replication (Moreno-Altamirano et al, Front Cell Infect Microbiol, 2019, vol 9, article 95), and as a result of this process, protons are released which reduces the pH of the host cell.

The infection process of many airborne viruses other than coronaviruses is also pH-dependent, for example, that of influenza viruses (Seth et al, J Virol, 2003, vol 77(11), pages 6520-6527; Seth et al, J Virol, 2003, vol 77(1), pages 167-178; Tong et al, Virology, 2002, vol 301, pages 322-333; Skehel et al, Annu Rev Biochem, 2000, vol 69, pages 531-569), Measles virus (Weiss et al, Am J Biochem & Biotech, 2013, vol 9(3), pages 243-254), Rubella virus (Lee et al, Clin Microbiol Rev, 2000, vol 13(4), pages 571-587), Parvovirus B19 (Blümel et al, Transfus Med Hemother, 2010, vol 37(6), pages 339-350), adeno-associated viruses (Salganik et al, J Virol, 2012, vol 86(21), pages 11877-11885), adenoviruses (Baker et al, Sci Adv, 2019, vol 5(9), eaax3567), herpes viruses such as Varicella-Zoster virus (Finnen et al, J Virol, 2006, vol 80(21), pages 10325-10334; Girsch et al, J Virol, 2019, vol 93(17), e00505-19), polyomaviruses such as WU polyomavirus (Bhattacharjee et al, Can J Microbiol, 2017, vol 63, pages 193-211), and Variola virus (Moss, Viruses, 2012, vol 4(5), pages 688-707).

Thus, the airborne virus infection process can be summarised as follows:
1. The airborne virus (such as an influenza virus, rhinovirus or coronavirus) is spread via water droplets or bodily fluids and enters the body via the nose passing to the nasal mucosa and also via the throat and eye.
2. The nasal mucosa is slightly acidic (pH 5.5-7.0) and can become more acidic due to acidic reflux from the stomach (GERD).
3. The protease enzymes found on the cells in the nasal epithelia operate optimally in highly acidic (pH<3) or highly alkaline (pH>9) environments, but the rate of hydrolysis is slower at physiological pH (7.2-7.38). The proteases are selective (and the cellular protease TMPRSS2 protease hydrolyses the coronavirus spike protein selectively).
4. The acidic environment of the nasal epithelia/cell interstitial fluid causes the spike protein to change its conformation, which becomes optimised for binding with the host cell membrane.
5. The viral spike protein binds to the cell membrane. In the case of influenza viruses, the hemagglutinin (HA) or neuraminidase (NA) protein binds to the sialic acids on the cell membrane. In the case of coronaviruses, the spike protein binds strongly to a dimer of ACE2 in the cell membrane.
6. Fusion of the virus with the cell takes place followed by endocytosis, and this requires an acidic environment (pH<5.5).
7. The viral cell coat opens and releases the viral nucleic acid into host cell as well as signalling proteins which disrupt the mitochondria and other processes to enable optimum nucleic acid transcription and production of daughter viruses.

Buffers have been used previously for inhalation and intranasal administration. For example, Diether et al (Arzneimittelforschung, 1982, vol 32(4), pages 406-408) studied the broncholytic effects of a phosphate and Tris (THAM) buffer administered by inhalation. Davis et al (Respiratory Care, 2013, vol 58(7), pages 1226-1232) studied the safety of an alkaline glycine buffer administered by inhalation with a view to potentially using the buffer as a drug delivery vehicle. Washington et al (Int J Pharm, 2000, vol 198(2), pages 139-146) determined the baseline human nasal pH and studied the effect of intranasally administered buffers on pH, using a sodium chloride (0.90%) buffer at pH 7.2 or 5.8, and a Sorensens phosphate buffer (0.06M or 0.13M) at pH 5.8 or 5.0. Gern et al (The Journal of Infectious Diseases, 2007, vol 195, pages 1137-1143) found that intranasal administration of a low pH citrate/phosphate buffer transiently lowered the surface pH of the human nasopharynx to pH ˜4.0, and inhibited replication of most human rhinoviruses and reduced the replication of influenza viruses, although respiratory symptoms were not significantly reduced. However, to date, buffers with a neutral pH have not been used to reduce the replication of airborne viruses.

### SUMMARY OF THE INVENTION

A first aspect of the invention provides a composition comprising one or more of clove oil, eucalyptus oil, basil oil, ginger oil, an extract or component of any of these, or a combination thereof, for use in the treatment, prophylactic treatment or amelioration of an airborne viral infection, or for use in reducing or preventing viral replication in a subject infected with an airborne virus or exposed to an airborne virus capable of causing an airborne viral infection in the subject.

In one embodiment of the first aspect of the invention, the composition further comprises a buffer; typically the composition comprises from 1 to 500 mmoles/L of a buffer.

In one embodiment of the first aspect of the invention, the composition is a solution, suspension, dispersion, mixture or emulsion, or the composition comprises microdroplets. In one embodiment of the first aspect of the invention, the composition is an aqueous composition. In one embodiment, the composition is an aqueous solution, an aqueous suspension, an aqueous dispersion, an aqueous mixture or an aqueous emulsion, or is an aqueous composition comprising microdroplets. In one embodiment, the composition is an aqueous emulsion or an aqueous composition comprising microdroplets.

A second aspect of the invention provides an aqueous composition comprising:

| | | |
|---|---|---|
| (i) | from 1 to 500 mmoles/L | buffer; and |
| (ii) | from 0.1 to 200 g/L | essential oil, or an extract or component |
| | | thereof, with antiviral, antibacterial or |
| | | antifungal activity. |

In one embodiment of the second aspect of the invention, the composition is for use in the treatment, prophylactic treatment or amelioration of an airborne viral infection, or for use in reducing or preventing viral replication in a subject infected with an airborne virus or exposed to an airborne virus capable of causing an airborne viral infection in the subject.

In one embodiment of the first or second aspect of the invention, the composition is for use in the treatment, prophylactic treatment or amelioration of an airborne viral infection.

In another embodiment of the first or second aspect of the invention, the composition is for use in reducing or preventing (typically reducing) viral replication in a subject infected with an airborne virus.

In a further embodiment of the first or second aspect of the invention, the composition is for use in reducing or preventing (typically reducing) viral replication in a subject exposed to an airborne virus capable of causing an airborne viral infection in the subject.

In one embodiment of the first or second aspect of the invention, the airborne viral infection is caused by a coronavirus selected from MERS-CoV, SARS-CoV, and SARS-CoV-2; typically the airborne viral infection is caused by SARS-CoV-2.

In another embodiment of the first or second aspect of the invention, the airborne viral infection is caused by an influenza virus selected from influenza A virus, influenza B virus, influenza C virus, and parainfluenza virus; typically the airborne viral infection is caused by influenza A virus (such as H1N1).

In one embodiment of the second aspect of the invention, the essential oil is selected from clove oil, eucalyptus oil, basil oil, ginger oil, saffron oil, cannabis oil, tea tree oil, black seed oil, an extract or component of any of these, or a combination thereof. Preferably the essential oil is selected from clove oil, eucalyptus oil, basil oil, ginger oil, an extract or component of any of these, or a combination thereof.

In one embodiment of the first or second aspect of the invention, the oil is clove oil (Syzygium aromaticum). Typically clove oil is extracted from clove flower buds, typically by steam distillation with water. Components of clove oil include eugenol, caryophyllene, ursolic acid, oleic acid (18:1 n-9), and linoleic acid (18:2 n-6).

In one embodiment of the first or second aspect of the invention, the oil is eucalyptus oil (Eucalyptus globulus). Typically eucalyptus oil is extracted from eucalyptus leaves, typically by steam distillation with water. Components of eucalyptus oil include eucalyptol, jensenone, ursolic acid, 1,8-cineole, α-pinene, palmitoleic acid (16:1 n-7), heptadecenoic acid (17:1 n-7), oleic acid (18:1 n-9), and linoleic acid (18:2 n-6).

In one embodiment of the first or second aspect of the invention, the oil is basil oil (Ocimum basilicum). Typically basil oil is extracted from basil leaves or seeds (typically basil leaves), typically with water, methanol or ethanol (typically by steam distillation with water). Components of basil oil include eugenol, caryophyllene, estragole, 1,8-cineole, camphor, thymol, eugenol epoxide, apigenin, linalool, rosmarinic acid, ursolic acid, oleic acid (18:1 n-9), linoleic acid (18:2 n-6), and α-linolenic acid (18:3 n-3).

In one embodiment of the first or second aspect of the invention, the oil is ginger oil (Zingiber officinale). Typically ginger oil is extracted from ginger rhizome, typically by steam distillation with water. Components of ginger oil include zingiberene, 6-gingerol, 6-shogaol, gingeronone A, ar-curcumene, β-sesquiphellandrene, α-farnesene, α-phellandrene, β-phellandrene, camphene, β-bisabolene, ursolic acid, palmitoleic acid (16:1 n-7), oleic acid (18:1 n-9), linoleic acid (18:2 n-6), α-linolenic acid (18:3 n-3), arachidonic acid (20:4 n-6), and erucic acid (22:1 n-9).

In one embodiment of the second aspect of the invention, the oil is saffron oil (Crocus sativus). Typically saffron oil is extracted from saffron flowers and stigmas. Components of saffron oil include crocetin, crocin, picrocrocin, safranal, and linoleic acid (18:2 n-6).

In one embodiment of the second aspect of the invention, the oil is cannabis oil (Cannabis sativa). A preferred cannabis strain is the Jamaican black swan strain. Typically cannabis oil is extracted from cannabis leaves. Components of cannabis oil include caflanone, tetrahydrocannabinol, cannabidiol, cannabinol, tetrahydrocannabivarin, linoleic acid (18:2 n-6), α-linolenic acid (18:3 n-3), and γ-linolenic acid 18:3 (n-6).

In one embodiment of the second aspect of the invention, the oil is tea tree oil (Melaleuca alternifolia). Typically tea tree oil is extracted from the leaves of the tea tree. Components of tea tree oil include 1,8-cineole, terpinen-4-ol, terpinolene, α-terpinene, γ-terpinene, p-cymene, α-pinene, α-terpineol, oleic acid (18:1 n-9), and linoleic acid (18:2 n-6).

In one embodiment of the second aspect of the invention, the oil is black seed oil (Nigella sativa). Typically black seed oil is extracted from the seeds of Nigella sativa. Components of black seed oil include thymoquinone, oleic acid (18:1 n-9), and linoleic acid (18:2 n-6).

In one embodiment of the first or second aspect of the invention, the extract or component of the essential oil is selected from eugenol, zingiberene, eucalyptol, jensenone, ursolic acid, caryophyllene, estragole, 1,8-cineole, camphor, thymol, eugenol epoxide, apigenin, linalool, rosmarinic acid, 6-gingerol, 6-shogaol, gingeronone A, crocetin, crocin, picrocrocin, safranal, caflanone, tetrahydrocannabinol, cannabidiol (or its metabolite 7-hydroxycannabidiol), cannabinol, tetrahydrocannabivarin, terpinen-4-ol, terpinolene, α-terpineol, thymoquinone, ar-curcumene, β-sesquiphellandrene, α-farnesene, α-phellandrene, β-phellandrene, camphene, β-bisabolene, α-terpinene, γ-terpinene, p-cymene, α-pinene, or a mono- or polyunsaturated fatty acid such as palmitoleic acid, heptadecenoic acid, oleic acid, linoleic acid, α-linolenic acid, γ-linolenic acid, arachidonic acid, or erucic acid.

In one embodiment of the first or second aspect of the invention, the essential oil, or the extract or component thereof, has antiviral activity.

In one embodiment of the first or second aspect of the invention, the buffer comprises hydrogen carbonate ions or an equivalent thereof.

In another embodiment of the first or second aspect of the invention, the buffer comprises, consists of or consists essentially of (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; and (ii) hydrogen carbonate ions or an equivalent thereof. Typically the buffer comprises, consists of or consists essentially of:

| | | |
|---|---|---|
| (i) | from 1 to 100 mmoles/L | Good's buffer, aminosulphonic acid, |
| | | aminosulphinic acid, phosphate, phosphite, heteroaryl, |
| | | phenolic acid, amino acid, peptide, peptide equivalent, |
| | | polymeric buffer, urea, ionic liquid buffer, or a |
| | | combination thereof; and |
| (ii) | from 21 to 35 mmoles/L | hydrogen carbonate ions or an equivalent thereof. |

In another embodiment of the first or second aspect of the invention, the buffer comprises, consists of or consists essentially of (i) one or more phosphates, and (ii) hydrogen carbonate ions. Typically the buffer comprises, consists of or consists essentially of KH₂PO₄, Na₂HPO₄ and NaHCO₃.

In one embodiment of the first or second aspect of the invention, the composition further comprises from 0.1 to 200 μmoles/L zinc ions.

In one embodiment of the first or second aspect of the invention, the composition further comprises from 1 to 100 μmoles/L transferrin and/or iron ions.

In one embodiment of the first or second aspect of the invention, the composition comprises:

| | | |
|---|---|---|
| (i) | from 1 to 500 mmoles/L | buffer; |
| (ii) | from 0.1 to 50 g/L | clove oil or an extract or component thereof; |
| (iii) | from 0.1 to 50 g/L | eucalyptus oil or an extract or component thereof; |
| (iv) | from 0.1 to 50 g/L | basil oil or an extract or component thereof; and |
| (v) | from 0.1 to 50 g/L | ginger oil or an extract or component thereof. |

In another embodiment of the first or second aspect of the invention, the composition comprises:

| | | |
|---|---|---|
| (i) | from 1 to 500 mmoles/L | buffer; |
| (ii) | from 0.1 to 200 μmoles/L | zinc ions; |
| (iii) | from 0.1 to 50 g/L | clove oil or an extract or component thereof; |
| (iv) | from 0.1 to 50 g/L | eucalyptus oil or an extract or component thereof; |
| (v) | from 0.1 to 50 g/L | basil oil or an extract or component thereof; and |
| (vi) | from 0.1 to 50 g/L | ginger oil or an extract or component thereof. |

In another embodiment of the first or second aspect of the invention, the composition comprises:

| (i) | from 25 to 250 mmoles/L | one or more phosphates; |
|---|---|---|
| (ii) | from 1 to 45 mmoles/L (preferably from 21 to 35 mmoles/L) hydrogen | |
| | | carbonate ions; |
| (iii) | from 0.1 to 10 g/L | clove oil or an extract or component thereof; |
| (iv) | from 0.1 to 10 g/L | eucalyptus oil or an extract or component thereof; |
| (v) | from 0.1 to 10 g/L | basil oil or an extract or component thereof; and |
| (vi) | from 0.1 to 10 g/L | ginger oil or an extract or component thereof. |

In another embodiment of the first or second aspect of the invention, the composition comprises:

| (i) | from 25 to 250 mmoles/L | one or more phosphates; |
|---|---|---|
| (ii) | from 1 to 45 mmoles/L (preferably from 21 to 35 mmoles/L) hydrogen | |
| | | carbonate ions; |
| (iii) | from 0.1 to 50 μmoles/L | zinc ions; |
| (iv) | from 0.1 to 10 g/L | clove oil or an extract or component thereof; |
| (v) | from 0.1 to 10 g/L | eucalyptus oil or an extract or component thereof; |
| (vi) | from 0.1 to 10 g/L | basil oil or an extract or component thereof; and |
| (vii) | from 0.1 to 10 g/L | ginger oil or an extract or component thereof. |

In one embodiment of the first or second aspect of the invention, the composition further comprises:

| (i) | calcium ions and magnesium ions at a molar concentration ratio of from 5:1 to | |
|---|---|---|
| | 1:1, wherein said calcium ions are at a concentration of from 0.1 to 2.5 | |
| | mmoles/L; | |
| (ii) | from 2.5 to 20 mmoles/L | potassium ions; |
| (iii) | from 96 to 126 mmoles/L | chloride ions; and |
| (iv) | from 100 to 300 mmoles/L | sodium ions. |

In one embodiment of the first or second aspect of the invention, the composition further comprises one or more additional components selected from NaCl, KCl, MgCl₂, xylitol, EDTA, CaCl₂, glycerol, HPMC, PEG 400, poloxamer 188, benzalkonium chloride and sodium hyaluronate.

In one embodiment of the first or second aspect of the invention, the composition has a pH of from 6.7 to 7.9 at a temperature of 37° C.

In one embodiment of the first or second aspect of the invention, the composition is an aqueous solution, an aqueous suspension, an aqueous dispersion, an aqueous mixture or an aqueous emulsion, or is an aqueous composition comprising microdroplets. In one embodiment, the composition is an aqueous emulsion or an aqueous composition comprising microdroplets.

A third aspect of the invention provides a buffer composition having a pH of from 6.7 to 7.9 at a temperature of 37° C., for use in the treatment, prophylactic treatment or amelioration of an airborne viral infection, or for use in reducing or preventing viral replication in a subject infected with an airborne virus or exposed to an airborne virus capable of causing an airborne viral infection in the subject.

In one embodiment of the third aspect of the invention, the buffer composition is for use in the treatment, prophylactic treatment or amelioration of an airborne viral infection.

In another embodiment of the third aspect of the invention, the buffer composition is for use in reducing or preventing (typically reducing) viral replication in a subject infected with an airborne virus.

In a further embodiment of the third aspect of the invention, the buffer composition is for use in reducing or preventing (typically reducing) viral replication in a subject exposed to an airborne virus capable of causing an airborne viral infection in the subject.

In one embodiment of the third aspect of the invention, the buffer composition is a solution, suspension, dispersion, mixture or emulsion, or the buffer composition comprises microdroplets. In one embodiment of the third aspect of the invention, the buffer composition is an aqueous composition. In one embodiment, the buffer composition is an aqueous solution, an aqueous suspension, an aqueous dispersion, an aqueous mixture or an aqueous emulsion, or is an aqueous composition comprising microdroplets.

In one embodiment of the third aspect of the invention, the buffer composition comprises a Good's buffer, an N-substituted aminosulphonic acid (such as BES), an N-unsubstituted aminosulphonic acid (such as taurine), an aminosulphinic acid, a phosphate, a phosphite, a heteroaryl (such as imidazole), a phenolic acid, an amino acid (such as proline), a peptide, a peptide equivalent, a polymeric buffer, a urea, an ionic liquid buffer, or a combination thereof.

A fourth aspect of the invention provides an aqueous composition comprising (i) a Good's buffer, an N-substituted aminosulphonic acid (such as BES), an N-unsubstituted aminosulphonic acid (such as taurine), an aminosulphinic acid, a phosphate, a phosphite, a heteroaryl (such as imidazole), a phenolic acid, an amino acid (such as proline), a peptide, a peptide equivalent, a polymeric buffer, a urea, an ionic liquid buffer, or a combination thereof; and (ii) hydrogen carbonate ions or an equivalent thereof; wherein the aqueous composition is for use in the treatment, prophylactic treatment or amelioration of an airborne viral infection, or for use in reducing or preventing viral replication in a subject infected with an airborne virus or exposed to an airborne virus capable of causing an airborne viral infection in the subject.

Typically, the aqueous composition of the fourth aspect of the invention is also a buffer composition of the third aspect of the invention.

One embodiment of the first, second, third or fourth aspect of the invention provides an aqueous composition comprising (i) an N-substituted aminosulphonic acid (such as BES); and (ii) hydrogen carbonate ions or an equivalent thereof.

One embodiment of the first, second, third or fourth aspect of the invention provides an aqueous composition comprising (i) an N-substituted aminosulphonic acid (such as BES); and (ii) hydrogen carbonate ions.

Another embodiment of the first, second, third or fourth aspect of the invention provides an aqueous composition comprising (i) an N-unsubstituted aminosulphonic acid (such as taurine); and (ii) hydrogen carbonate ions or an equivalent thereof.

Another embodiment of the first, second, third or fourth aspect of the invention provides an aqueous composition comprising (i) an aminosulphinic acid; and (ii) hydrogen carbonate ions or an equivalent thereof.

Another embodiment of the first, second, third or fourth aspect of the invention provides an aqueous composition comprising (i) a phosphate; and (ii) hydrogen carbonate ions or an equivalent thereof.

Another embodiment of the first, second, third or fourth aspect of the invention provides an aqueous composition comprising (i) a phosphite; and (ii) hydrogen carbonate ions or an equivalent thereof.

Another embodiment of the first, second, third or fourth aspect of the invention provides an aqueous composition comprising (i) a heteroaryl (such as imidazole); and (ii) hydrogen carbonate ions or an equivalent thereof.

Another embodiment of the first, second, third or fourth aspect of the invention provides an aqueous composition comprising (i) a phenolic acid; and (ii) hydrogen carbonate ions or an equivalent thereof.

Another embodiment of the first, second, third or fourth aspect of the invention provides an aqueous composition comprising (i) an amino acid (such as proline); and (ii) hydrogen carbonate ions or an equivalent thereof.

Another embodiment of the first, second, third or fourth aspect of the invention provides an aqueous composition comprising (i) a peptide or a peptide equivalent; and (ii) hydrogen carbonate ions or an equivalent thereof.

Another embodiment of the first, second, third or fourth aspect of the invention provides an aqueous composition comprising (i) a polymeric buffer; and (ii) hydrogen carbonate ions or an equivalent thereof.

Another embodiment of the first, second, third or fourth aspect of the invention provides an aqueous composition comprising (i) a urea; and (ii) hydrogen carbonate ions or an equivalent thereof.

Another embodiment of the first, second, third or fourth aspect of the invention provides an aqueous composition comprising (i) an ionic liquid buffer; and (ii) hydrogen carbonate ions or an equivalent thereof.

In one embodiment of the fourth aspect of the invention, the aqueous composition is for use in the treatment, prophylactic treatment or amelioration of an airborne viral infection.

In another embodiment of the fourth aspect of the invention, the aqueous composition is for use in reducing or preventing (typically reducing) viral replication in a subject infected with an airborne virus.

In a further embodiment of the fourth aspect of the invention, the aqueous composition is for use in reducing or preventing (typically reducing) viral replication in a subject exposed to an airborne virus capable of causing an airborne viral infection in the subject.

In one embodiment of the first, second, third or fourth aspect of the invention, the composition is an aqueous composition comprising:
(i) from 1 to 100 mmoles/L (preferably from 1 to 12 mmoles/L) Good's buffer, aminosulphonic acid, aminosulphinic acid, phosphate, phosphite, heteroaryl, phenolic acid, amino acid, peptide, peptide equivalent, polymeric buffer, urea, ionic liquid buffer, or a combination thereof;
(ii) calcium ions and magnesium ions at a molar concentration ratio of from 5:1 to 1:1, wherein said calcium ions are at a concentration of from 0.1 to 2.5 mmoles/L;

| | | |
|---|---|---|
| (iii) | from 21 to 35 mmoles/L | hydrogen carbonate ions or an equivalent thereof; |
| (iv) | from 2.5 to 6.2 mmoles/L | potassium ions; |
| (v) | from 96 to 126 mmoles/L | chloride ions; and |
| (vi) | from 100 to 150 mmoles/L | sodium ions. |

In one embodiment of the first, second, third or fourth aspect of the invention, the composition is an aqueous composition comprising:

| (i) | from 1 to 12 mmoles/L | N-substituted aminosulphonic acid; |
|---|---|---|
| (ii) | calcium ions and magnesium ions at a molar concentration ratio of from 5:1 to | |
| | 1:1, wherein said calcium ions are at a concentration of from 0.1 to 2.5 | |
| | mmoles/L; | |
| (iii) | from 21 to 35 mmoles/L | hydrogen carbonate ions; |
| (iv) | from 2.5 to 6.2 mmoles/L | potassium ions; |
| (v) | from 96 to 126 mmoles/L | chloride ions; and |
| (vi) | from 100 to 150 mmoles/L | sodium ions. |

In one embodiment of the first, second, third or fourth aspect of the invention, the composition further comprises zinc ions. For example, the composition of the first, second, third or fourth aspect of the invention may be a composition (such as an aqueous composition) comprising from 0.1 to 200 μmoles/L zinc ions. Without wishing to be bound by theory, it is currently believed that zinc ions present in the composition may disrupt the activity of ACE2 and may prevent airborne viruses from binding to host cell membranes. It is also currently believed that zinc ions present in the composition may impair the replication of viruses, in particular coronaviruses and influenza viruses.

Zinc ions are antiviral on several levels. Apart from being important for the mucociliar clearance of viruses (Zanin et al, Cell Host Microbe, 2016, vol 19(2), pages 159-168), zinc ions directly inhibit cytoplasmic viral replication that is mediated by the viral RNA-dependent RNA polymerase (te Velthuis et al, PLoS Pathog, 2010, vol 6(11), e1001176). RNA-dependent RNA polymerase is required for the production of new viral genomes as well as sub-genomic RNAs which are needed for the translation and production of viral structural proteins. A further step in the viral life cycle requires Mpro, a protease involved in the processing of viral structural proteins during assembly. Mpro is also inhibited in the presence of zinc ions (Hsu et al, FEBS Lett, 2004, vol 574(1-3), pages 116-120). The levels of pro-inflammatory cytokines and reactive oxygen species (ROS) (Wessels et al, Nutrients, 2017, vol 9(12), 1286) and inhibition of viral uncoating (Read et al, Adv Nutr, 2019, vol 10(4), pages 696-710) are also reported to be influenced by the levels of zinc ions.

In one embodiment of the first, second, third or fourth aspect of the invention, the composition further comprises transferrin, such as holo-transferrin, apotransferrin or mono ferric transferrin. For example, the composition of the first, second, third or fourth aspect of the invention may be a composition (such as an aqueous composition) comprising from 1 to 100 μmoles/L transferrin.

In one embodiment of the first, second, third or fourth aspect of the invention, the composition further comprises iron ions, such as Fe²⁺ or Fe³⁺. For example, the composition of the first, second, third or fourth aspect of the invention may be a composition (such as an aqueous composition) comprising from 1 to 100 μmoles/L iron ions.

In one embodiment of the first, second, third or fourth aspect of the invention, the composition further comprises transferrin and iron ions. For example, the composition of the first, second, third or fourth aspect of the invention may be a composition (such as an aqueous composition) comprising from 1 to 100 μmoles/L transferrin and from 1 to 100 μmoles/L iron ions. Without wishing to be bound by theory, it is currently believed that transferrin and iron ions present in the composition may aid in regulating the pH of the interstitial fluid and thereby increase the effectiveness of the composition.

A fifth aspect of the invention provides an aqueous composition comprising (i) a Good's buffer, an N-substituted aminosulphonic acid (such as BES), an N-unsubstituted aminosulphonic acid (such as taurine), an aminosulphinic acid, a phosphate, a phosphite, a heteroaryl (such as imidazole), a phenolic acid, an amino acid (such as proline), a peptide, a peptide equivalent, a polymeric buffer, a urea, an ionic liquid buffer, or a combination thereof; (ii) hydrogen carbonate ions or an equivalent thereof; and (iii) zinc ions.

Typically, the aqueous composition of the fifth aspect of the invention is also a composition of the third and/or fourth aspect of the invention.

One embodiment of the first, second, third, fourth or fifth aspect of the invention provides an aqueous composition comprising (i) an N-substituted aminosulphonic acid (such as BES); (ii) hydrogen carbonate ions or an equivalent thereof; and (iii) zinc ions.

One embodiment of the first, second, third, fourth or fifth aspect of the invention provides an aqueous composition comprising (i) an N-substituted aminosulphonic acid (such as BES); (ii) hydrogen carbonate ions; and (iii) zinc ions.

Another embodiment of the first, second, third, fourth or fifth aspect of the invention provides an aqueous composition comprising (i) an N-unsubstituted aminosulphonic acid (such as taurine); (ii) hydrogen carbonate ions or an equivalent thereof; and (iii) zinc ions.

Another embodiment of the first, second, third, fourth or fifth aspect of the invention provides an aqueous composition comprising (i) an aminosulphinic acid; (ii) hydrogen carbonate ions or an equivalent thereof; and (iii) zinc ions.

Another embodiment of the first, second, third, fourth or fifth aspect of the invention provides an aqueous composition comprising (i) a phosphate; (ii) hydrogen carbonate ions or an equivalent thereof; and (iii) zinc ions.

Another embodiment of the first, second, third, fourth or fifth aspect of the invention provides an aqueous composition comprising (i) a phosphite; (ii) hydrogen carbonate ions or an equivalent thereof; and (iii) zinc ions.

Another embodiment of the first, second, third, fourth or fifth aspect of the invention provides an aqueous composition comprising (i) a heteroaryl (such as imidazole); (ii) hydrogen carbonate ions or an equivalent thereof; and (iii) zinc ions.

Another embodiment of the first, second, third, fourth or fifth aspect of the invention provides an aqueous composition comprising (i) a phenolic acid; (ii) hydrogen carbonate ions or an equivalent thereof; and (iii) zinc ions.

Another embodiment of the first, second, third, fourth or fifth aspect of the invention provides an aqueous composition comprising (i) an amino acid (such as proline); (ii) hydrogen carbonate ions or an equivalent thereof; and (iii) zinc ions.

Another embodiment of the first, second, third, fourth or fifth aspect of the invention provides an aqueous composition comprising (i) a peptide or a peptide equivalent; (ii) hydrogen carbonate ions or an equivalent thereof; and (iii) zinc ions.

Another embodiment of the first, second, third, fourth or fifth aspect of the invention provides an aqueous composition comprising (i) a polymeric buffer; (ii) hydrogen carbonate ions or an equivalent thereof; and (iii) zinc ions.

Another embodiment of the first, second, third, fourth or fifth aspect of the invention provides an aqueous composition comprising (i) a urea; (ii) hydrogen carbonate ions or an equivalent thereof; and (iii) zinc ions.

Another embodiment of the first, second, third, fourth or fifth aspect of the invention provides an aqueous composition comprising (i) an ionic liquid buffer; (ii) hydrogen carbonate ions or an equivalent thereof; and (iii) zinc ions.

In one embodiment of the first, second, third, fourth or fifth aspect of the invention, the composition is an aqueous composition comprising:

| (i) | from 1 to 100 mmoles/L (preferably from 1 to 12 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | from 21 to 35 mmoles/L | hydrogen carbonate ions or an equivalent thereof; |
| | and | |
| (iii) | from 0.1 to 200 μmoles/L | zinc ions. |

In one embodiment of the first, second, third, fourth or fifth aspect of the invention, the composition is an aqueous composition comprising:

| | | |
|---|---|---|
| (i) | from 1 to 12 mmoles/L | N-substituted aminosulphonic acid; |
| (ii) | from 21 to 35 mmoles/L | hydrogen carbonate ions; and |
| (iii) | from 0.1 to 200 μmoles/L | zinc ions. |

In one embodiment of the first, second, third, fourth or fifth aspect of the invention, the composition is an aqueous composition comprising:

| (i) | from 1 to 100 mmoles/L (preferably from 1 to 12 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | calcium ions and magnesium ions at a molar concentration ratio of from 5:1 to | |
| | 1:1, wherein said calcium ions are at a concentration of from 0.1 to 2.5 | |
| | mmoles/L; | |
| (iii) | from 21 to 35 mmoles/L | hydrogen carbonate ions or an equivalent thereof; |
| (iv) | from 2.5 to 6.2 mmoles/L | potassium ions; |
| (v) | from 96 to 126 mmoles/L | chloride ions; |
| (vi) | from 100 to 150 mmoles/L | sodium ions; and |
| (vii) | from 0.1 to 200 μmoles/L | zinc ions. |

In one embodiment of the first, second, third, fourth or fifth aspect of the invention, the composition is an aqueous composition comprising:

| (ii) | calcium ions and magnesium ions at a molar concentration ratio of from 5:1 to | |
|---|---|---|
| | 1:1, wherein said calcium ions are at a concentration of from 0.1 to 2.5 | |
| | mmoles/L; | |
| (iii) | from 21 to 35 mmoles/L | hydrogen carbonate ions; |
| (iv) | from 2.5 to 6.2 mmoles/L | potassium ions; |
| (v) | from 96 to 126 mmoles/L | chloride ions; |
| (vi) | from 100 to 150 mmoles/L | sodium ions; and |
| (vii) | from 0.1 to 200 μmoles/L | zinc ions. |

The fifth aspect of the invention also provides an aqueous composition comprising (i) a Good's buffer, an N-substituted aminosulphonic acid (such as BES), an N-unsubstituted aminosulphonic acid (such as taurine), an aminosulphinic acid, a phosphate, a phosphite, a heteroaryl (such as imidazole), a phenolic acid, an amino acid (such as proline), a peptide, a peptide equivalent, a polymeric buffer, a urea, an ionic liquid buffer, or a combination thereof; (ii) hydrogen carbonate ions or an equivalent thereof; and (iii) transferrin and/or iron ions.

In one embodiment of the first, second, third, fourth or fifth aspect of the invention, the composition is an aqueous composition comprising:

| (i) | from 1 to 100 mmoles/L (preferably from 1 to 12 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | from 21 to 35 mmoles/L | hydrogen carbonate ions or an equivalent thereof; |
| (iii) | from 1 to 100 μmoles/L | transferrin; and |
| (vi) | from 1 to 100 μmoles/L | iron ions. |

In one embodiment of the first, second, third, fourth or fifth aspect of the invention, the composition is an aqueous composition comprising:

| | | |
|---|---|---|
| (i) | from 1 to 12 mmoles/L | N-substituted aminosulphonic acid; |
| (ii) | from 21 to 35 mmoles/L | hydrogen carbonate ions; |
| (iii) | from 1 to 100 μmoles/L | transferrin; and |
| (vi) | from 1 to 100 μmoles/L | iron ions. |

In one embodiment of the first, second, third, fourth or fifth aspect of the invention, the composition is an aqueous composition comprising:

| (i) | from 1 to 100 mmoles/L (preferably from 1 to 12 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | calcium ions and magnesium ions at a molar concentration ratio of from 5:1 to | |
| | 1:1, wherein said calcium ions are at a concentration of from 0.1 to 2.5 | |
| | mmoles/L; | |
| (iii) | from 21 to 35 mmoles/L | hydrogen carbonate ions or an equivalent thereof; |
| (iv) | from 2.5 to 6.2 mmoles/L | potassium ions; |
| (v) | from 96 to 126 mmoles/L | chloride ions; |
| (vi) | from 100 to 150 mmoles/L | sodium ions; |
| (vii) | from 1 to 100 μmoles/L | transferrin; and |
| (viii) | from 1 to 100 μmoles/L | iron ions. |

In one embodiment of the first, second, third, fourth or fifth aspect of the invention, the composition is an aqueous composition comprising:

| (i) | from 1 to 12 mmoles/L | N-substituted aminosulphonic acid; |
|---|---|---|
| (ii) | calcium ions and magnesium ions at a molar concentration ratio of from 5:1 to | |
| | 1:1, wherein said calcium ions are at a concentration of from 0.1 to 2.5 | |
| | mmoles/L; | |
| (iii) | from 21 to 35 mmoles/L | hydrogen carbonate ions; |
| (iv) | from 2.5 to 6.2 mmoles/L | potassium ions; |
| (v) | from 96 to 126 mmoles/L | chloride ions; |
| (vi) | from 100 to 150 mmoles/L | sodium ions; |
| (vii) | from 1 to 100 μmoles/L | transferrin; and |
| (viii) | from 1 to 100 μmoles/L | iron ions. |

In one embodiment of the first, second, third, fourth or fifth aspect of the invention, the composition comprises a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof. These buffers are described in more detail below. The buffers can be used as such or as a salt, such as a sodium or potassium salt. Preferably the composition comprises a Good's buffer, an aminosulphonic acid, an aminosulphinic acid, a phosphate, a phosphite, a heteroaryl, a phenolic acid, an amino acid, a peptide, a peptide equivalent, a polymeric buffer, an ionic liquid buffer, or a combination thereof.

Preferably the composition comprises a Good's buffer, an aminosulphonic acid, an aminosulphinic acid, a phosphate, a phosphite, a heteroaryl, an amino acid, a peptide, a peptide equivalent, an ionic liquid buffer, or a combination thereof. Preferably the composition further comprises hydrogen carbonate ions or an equivalent thereof; preferably hydrogen carbonate ions.

Goods Buffers

In one embodiment of the first, second, third, fourth or fifth aspect of the invention, the composition comprises a Good's buffer. In one embodiment, the Good's buffer is present at a concentration of from 1 to 100 mmoles/L, or at a concentration of from 1 to 12 mmoles/L, or at a concentration of from 3 to 8 mmoles/L, or at a concentration of about 5 mmoles/L.

For the purposes of the present invention, all of the buffers listed in FIG. 1 are considered to be Good's buffers. For the purposes of the present invention, Good's buffers may be classified as N-substituted aminosulphonic acid Good's buffers and non-sulphonic acid Good's buffers, as indicated in FIG. 1.

Aminosulphonic Acid Buffers

In one embodiment of the first, second, third, fourth or fifth aspect of the invention, the composition comprises an aminosulphonic acid. In one embodiment, the aminosulphonic acid is present at a concentration of from 1 to 100 mmoles/L, or at a concentration of from 1 to 12 mmoles/L, or at a concentration of from 3 to 8 mmoles/L, or at a concentration of about 5 mmoles/L.

In one embodiment of the first, second, third, fourth or fifth aspect of the invention, the composition comprises an N-substituted aminosulphonic acid. In one embodiment, the N-substituted aminosulphonic acid is an N-substituted aminosulphonic acid Good's buffer (such as BES). In one embodiment, the N-substituted aminosulphonic acid is an N-substituted aminoalkylsulphonic acid, such as an N-substituted amino(C₁-C₄ alkyl)sulphonic acid (such as BES) or an N-substituted amino(C₁-C₄ hydroxyalkyl) sulphonic acid.

In one embodiment of the first, second, third, fourth or fifth aspect of the invention, the composition comprises an N-substituted aminosulphonic acid. In one embodiment, the N-substituted aminosulphonic acid is selected from ACES, AMPSO, BES, CABS, CAPS, CAPSO, CHES, DIPSO, HEPBS, HEPES, HEPPS, HEPPSO, MES, MOBS, MOPS, MOPSO, PIPES, POPSO, TABS, TAPS, TAPSO, TES, or a combination thereof. In one embodiment, the N-substituted aminosulphonic acid is selected from BES, TES, HEPES, PIPES, CAPS, or a combination thereof. In one embodiment, the N-substituted aminosulphonic acid is selected from BES, DIPSO, TES, TAPS, TAPSO, TABS, or a combination thereof. In one embodiment, the N-substituted aminosulphonic acid is selected from BES, DIPSO, or a combination thereof. In one embodiment, the N-substituted aminosulphonic acid is BES.

In one embodiment of the first, second, third, fourth or fifth aspect of the invention, the composition comprises an N-unsubstituted aminosulphonic acid. In one embodiment, the N-unsubstituted aminosulphonic acid is an N-unsubstituted aminoalkylsulphonic acid, such as an N-unsubstituted amino(C₁-C₄ alkyl)sulphonic acid (such as taurine) or an N-unsubstituted amino(C₁-C₄ hydroxyalkyl)sulphonic acid.

In one embodiment of the first, second, third, fourth or fifth aspect of the invention, the composition comprises an N-unsubstituted aminosulphonic acid. In one embodiment, the N-unsubstituted aminosulphonic acid is taurine [H₂N—CH₂CH₂—SO₃H], 3-aminopropane-1-sulphonic acid [H₂N—CH₂CH₂CH₂—SO₃H], 3-amino-2-hydroxypropane-1-sulphonic acid [H₂N—CH₂—CH(OH)—CH₂—SO₃H], 4-aminobutane-1-sulphonic acid [H₂N—CH₂CH₂CH₂CH₂—SO₃H], or aminobenzenesulphonic acid (including 2-, 3- and 4-aminobenzenesulphonic acid). In one embodiment, the N-unsubstituted aminosulphonic acid is taurine, or taurine bound to hyaluronic acid, or taurine bound to chitosan, or taurine bound to carrageenan. In one embodiment, the N-unsubstituted aminosulphonic acid is taurine.

In one embodiment of the first, second, third, fourth or fifth aspect of the invention, the aminosulphonic acid has the structure

R^{a}R^{b}N—(CR^{c}R^{d})ₘ—SO₃H (formula I)

wherein:
each -R^{a} is independently hydrogen, —R^{aaa}, —CHO, —COR^{aaa}, —CO₂R^{aaa}, —SOR^{aaa}, —SONH₂, —SONHR^{aaa}, —SON(R^{aaa})₂, —SO₂H, —SO₂R^{aaa}, —SO₂NH₂, —SO₂NHR^{aaa}, —SO₂N(R^{aaa})₂, —SO₃H, —SO₃R^{aaa}, —CONH₂, —CONHR^{aaa}, —CON(R^{aaa})₂, —C(NH)NH₂, —C(NH)NHR^{aaa}, —C(NH)N(R^{aaa})₂, —C(NR^{aaa})NHR^{aaa}, —C(NR^{aaa})N(R^{aaa})₂, -R^{aa}-R^{aaa}, —R^{aa}—OR^{aaa}, —R^{aa}—SR^{aaa}, —R^{aa}—OH, —R^{aa}—SH, —R^{aa}—CHO, —R^{aa}—COR^{aaa}, —R^{aa}—CO₂H, —R^{aa}—CO₂R^{aaa}, —R^{aa}—OCOR^{aaa}, —R^{aa}—SOR^{aaa}, —R^{aa}—SONH₂, —R^{aa}—SONHR^{aaa}, —R^{aa}—SON(R^{aaa})₂, —R^{aa}—SO₂H, —R^{aa}—SO₂R^{aaa}, —R^{aa}—SO₂NH₂, —R^{aa}—SO₂NHR^{aaa}, —R^{aa}—SO₂N(R^{aaa})₂, —R^{aa}—SO₃H, —R^{aa}—SOR^{aaa}, —R^{aa}—NH₂, —R^{aa}—NHR^{aaa}, —R^{aa}—N(R^{aaa})₂, —R^{aa}—CONH₂, —R^{aa}—CONHR^{aaa}, —R^{aa}—CON(R^{aaa})₂, —R^{aa}—C(NH)NH₂, —R^{aa}—C(NH)NHR^{aaa}, —R^{aa}—C(NH)N(R^{aaa})₂, —R^{aa}—C(NR^{aaa})NHR^{aaa}, —R^{aa}—C(NR^{aaa})N(R^{aaa})₂, —R^{aaaa}—(R^{aa}O)ₚ—H, —R^{aaaa}—(R^{aa}O)ₚ—R^{aaa}, —R^{aaaa}—(R^{aa}O)ₚ—COR^{aaa}, —R^{aaaa}—(R^{aa}NR^{x})ₚ—H, —R^{aaaa}—(R^{aa}NR^{x})ₚ—R^{aaa}, —R^{aaaa}—(R^{aa}NR^{x})ₚ—COR^{aaa}, —R^{aaaa}—(R^{aa}NR^{x})ₚ—CONH₂, —R^{aaaa}—(R^{aa}NR^{x})ₚ—CONHR^{aaa}, —R^{aaaa}—(R^{aa}NR^{x})ₚ—CON(R^{aaa})₂, —R^{aaaa}—(R^{aa}NR^{x})ₚ—C(NH)NH₂, —R^{aaaa}—(R^{aa}NR^{x})ₚ—C(NH)NHR^{aaa}, —R^{aaaa}—(R^{aa}NR^{x})ₚ—C(NH)N(R^{aaa})₂, —R^{aaaa}—(R^{aa}NR^{x})ₚ—C(NR^{aaa})NHR^{aaa}, or —R^{aaaa}—(R^{aa}NR^{x})ₚ—C(NR^{aaa})N(R^{aaa})₂;
each -R^{aa}- is independently C₁-C₄ alkylene or C₁-C₄ haloalkylene;
each -R^{aaa} is independently a C₁-C₃₀ alkyl (preferably C₁-C₁₂ alkyl or C₁-C₆ alkyl), C₂-C₃, alkenyl (preferably C₂-C₁₂ alkenyl or C₂-C₆ alkenyl), C₂-C₃₀ alkynyl (preferably C₂-C₁₂ alkynyl or C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SOH, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups;
each -R^{aaaa}- is independently a bond, —CO—, —CO₂—, —CONH—, —CONR^{x}—, —R^{aa}—CO—, —R^{aa}—CO₂—, —R^{aa}—CONH—, —R^{aa}—CONR^{x}—, —R^{aa}—O—, —R^{aa}—NH—, or —R^{aa}—NR^{x}—;
-R^{b} is hydrogen, —R^{bbb}, —CHO, —COR^{bbb}, —CO₂R^{bbb}, —SOR^{bbb}, —SONH₂, —SONHR^{bbb}, —SON(R^{bbb})₂, —SO₂H, —SO₂R^{bbb}, —SO₂NH₂, —SO₂NHR^{bbb}, —SO₂N(R^{bbb})₂, —SO₃H, —SO₃R^{bbb}, —CONH₂, —CONHR^{bbb}, —CON(R^{bbb})₂, —C(NH)NH₂, —C(NH)NHR^{bbb}, —C(NH)N(R^{bbb})₂, —C(NR^{bbb})NHR^{bbb}, —C(NR^{bbb})N(R^{bbb})₂, —R^{bb}—R^{bbb}, —R^{bb}—OR^{bbb}, —R^{bb}—SR^{bbb}, —R^{bb}—OH, —R^{bb}—SH, —R^{bb}—CHO, —R^{bb}—COR^{bbb}, —R^{bb}—CO₂H, —R^{bb}—CO₂R^{bbb}, —R^{bb}—OCOR^{bbb}, —R^{bb}—SOR^{bbb}, —R^{bb}—SONH₂, —R^{bb}—SONHR^{bbb}, —R^{bb}—SON(R^{bbb})₂, —R^{bb}—SO₂H, —R^{bb}—SO₂R^{bbb}, —R^{bb}—SO₂NH₂, —R^{bb}—SO₂NHR^{bbb}, —R^{bb}—SO₂N(R^{bbb})₂, —R^{bb}—SO₃H, —R^{bb}—SO₃R^{bbb}, —R^{bb}—NH₂, —R^{bb}—NHR^{bbb}, —R^{bb}—N(R^{bbb})₂, —R^{bb}—CONH₂, —R^{bb}—CONHR^{bbb}, —R^{bb}—CON(R^{bbb})₂, —R^{bb}—C(NH)NH₂, —R^{bb}—C(NH)NHR^{bbb}, —R^{bb}—C(NH)N(R^{bbb})₂, —R^{bb}—C(NR^{bbb})NHR^{bbb}, —R^{bb}—C(NR^{bbb})N(R^{bbb})₂, —R^{bbbb}—(R^{bb}O)_{q}—H, —R^{bbbb}—(R^{bb}O)_{q}R^{bbb}, —R^{bbbb}—(R^{bb}O)_{q}—COR^{bbb}, —R^{bbbb}—(R^{bb}NR^{x})_{q}—H, —R^{bbbb}—(R^{bb}NR^{x})_{q}—R^{bbb}, —R^{bbbb}—(R^{bb}NR^{xx})_{q}—COR^{bbb}, —R^{bbbb}—(R^{bb}NR^{x})_{q}—CONH₂, —R^{bbbb}—(R^{bb}NR^{xx})_{q}—CONHR^{bbb}, —R^{bbbb}—(R^{bb}NR^{xx})_{q}—CON(R^{bbb})₂, —R^{bbbb}—(R^{bb}NR^{xx})_{q}—C(NH)NH₂, —R^{bbbb}—(R^{bb}NR^{xx})_{q}—C(NH)NHR^{bbb}, —R^{bbbb}—(R^{bb}NR^{xx})_{q}—C(NH)N(R^{bbb})₂, —R^{bbbb}—(R^{bb}NR—)_{q}—C(NR^{bbb})NHR^{bbb}, or —R^{bbbb}—(R^{bb}NR^{xx})_{q}—C(NR^{bbb})N(R^{bbb})₂ each -R^{bb}— is independently C₁-C₄ alkylene or C₁-C₄ haloalkylene;
each -R^{bbb} is independently a C₁-C₃₀ alkyl (preferably C₁-C₁₂ alkyl or C₁-C₆ alkyl), C₂-C₃₀ alkenyl (preferably C₂-C₁₂ alkenyl or C₂-C₆ alkenyl), C₂-C₃₀ alkynyl (preferably C₂-C₁₂ alkynyl or C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SOH, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups;
R^{bbbb}- is a bond, —CO—, —CO₂—, —CONH—, —CONR^{x}—, —R^{bb}—CO—, —R^{bb}—CO₂—, —R^{bb}—CONH—, —R^{bb}—CONR^{xx}—, —R^{bb}—O—, —R^{bb}—NH—, or —R^{bb}—NR^{xx}—;
or —R^{a} and —R^{b} together with the nitrogen atom to which they are attached, form a 4- to 7-membered heterocyclic group, which is optionally substituted with (i) one or more halo groups, and/or (ii) one, two, three, four or five substituents independently selected from —R^{a}, —OH, —SH, —NH₂, —CN, —CO₂H, —SO₂H, —SOH, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo groups and/or one, two, three, four or five substituents independently selected from —OH, —SH, —NH₂, —CN, —CO₂H, —SO₂H, —SOH, —CONH₂, —OP(O)(OH)₂, and —OPH(O)(OH);
each -R^{c} is independently hydrogen, halo, —R^{cc}—R^{ccc}, —R^{cc}—OR^{ccc}—R^{cc}—SR^{ccc}, —R^{cc}—OH, —R^{cc}—SH, —R^{cc}—CHO, —R^{cc}—COR^{ccc}, —R^{cc}—CO₂H, —R^{cc}—CO₂R^{ccc}, —R^{cc}—OCOR^{ccc}, —R^{cc}—SOR^{ccc}, —R^{cc}—SONH₂, —R^{cc}—SONHR^{ccc}, —R^{cc}—SON(R^{ccc})₂, —R^{cc}—SO₂H, —R^{cc}—SO₂R^{ccc}, —R^{cc}—SO₂NH₂, —R^{cc}—SO₂NHR^{ccc}, —R^{cc}—SO₂N(R^{ccc})₂, —R^{cc}—SO₃H, —R^{cc}—SO₃R^{ccc}, —R^{cc}—NH₂, —R^{cc}—NHR^{ccc}, —R^{cc}—N(R^{ccc})₂, —R^{cc}—CONH₂, —R^{cc}—CONHR^{ccc}, —R^{cc}—CON(R^{ccc})₂, —R^{cc}—C(NH)NH₂, —R^{cc}—C(NH)NHR^{ccc}, —R^{cc}—C(NH)N(R^{ccc})₂, —R^{cc}—C(NR^{ccc})NHR^{ccc}, —R^{cc}—C(NR^{cc})N(R^{ccc})₂, —R^{cccc}—(R^{cccc}O)ᵣ—H, —R^{cccc}—(R^{ccc}O)ᵣ—R^{ccc}, —R^{cccc}—(R^{cccc}O)ᵣ—COR^{ccc}, —R^{cccc}—(R^{ccc}NR^{y})ᵣ—H, —R^{cccc}—(R^{cccc}NR^{y})ᵣ—R^{ccc}, —R^{cccc}—(R^{cccc}NR^{y})ᵣ—COR^{ccc}, —R^{cccc}—(R^{ccccc}NR^{y})ᵣ—CONH₂, —R^{cccc}—(R^{cccc}NR^{y})ᵣ—CONHR^{ccc}, —R^{cccc}—(R^{ccccc}NR^{y})ᵣ—CON(R^{ccc})₂, —R^{ccc}—(R^{ccccc}NR^{y})ᵣ—C(NH)NH₂, —R^{cccc}—(R^{ccccc}NR^{y})ᵣ—C(NH)NHR^{ccc}, —R^{ccc}—(R^{ccc}NR^{y})ᵣ—C(NH)N(R^{ccc})₂, —R^{cccc}— (R^{ccc}NR^{y})ᵣ—C(NR^{cc})NHR^{ccc}, or —R^{cccc}—(R^{ccccc}NR^{y})ᵣ—C(NR^{cc})N(R^{ccc})₂;
each -R^{cc}- is independently a bond, C₁-C₆ alkylene, C₂-C₆ alkenylene, or —(CH₂)ₙ—(C₆H₄)—(CH₂)ₙ—, each of which is optionally substituted with one or more halo and/or —OH groups;
each -R^{ccc} is independently a C₁-C₁₂ alkyl (preferably C₁-C₆ alkyl), C₂-C₁₂ alkenyl (preferably C₂-C₆ alkenyl), C₂-C₁₂ alkynyl (preferably C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SO₃H, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups;
each -R^{cccc}- is independently a bond, —CO—, —CO₂—, —CONH—, —CONR^{y}—, —R^{ccccc}—CO—, —R^{ccccc}—CO₂—, —R^{ccccc}—CONH—, —R^{ccccc}—CONR^{y}—, —R^{ccccc}—O—, —R^{ccccc}—NH—, or —R^{ccccc}—NR^{y}—;
each -R^{ccccc}- is independently C₁-C₄ alkylene or C₁-C₄ haloalkylene;
and/or —R^{b} and any —R^{c} together with the atoms to which they are attached, form a 4- to 7-membered heterocyclic group, each of which is optionally substituted with one or more halo groups and/or one, two, three, four or five substituents independently selected from C₁-C₄ alkyl, phenyl, benzyl, —O—(C₁-C₄ alkyl), —O—CO—(C₁-C₄ alkyl), —CO₂—(C₁-C₄ alkyl), —OH, —SH, —NH₂, —CN, —CO₂H, —SO₂H, —SO₃H, —CONH₂, —OP(O)(OH)₂, —OPH(O)(OH)₂, and oxo (═O), wherein each C₁-C₄ alkyl, phenyl or benzyl group is optionally substituted with one or more halo groups and/or one, two, three, four or five substituents independently selected from —OH, —SH, —NH₂, —CN, —CO₂H, —SO₂H, —SO₃H, —CONH₂, —OP(O)(OH)₂, and —OPH(O)(OH);
and/or any two —R^{c} together with the carbon atoms to which they are attached, form a C₄-C₇ cycloalkyl or 4- to 7-membered heterocyclic group, each of which is optionally substituted with one or more halo groups and/or one, two, three, four or five substituents independently selected from C₁-C₄ alkyl, phenyl, benzyl, —O—(C₁-C₄ alkyl), —O—CO—(C₁-C₄ alkyl), —CO₂—(C₁-C₄ alkyl), —OH, —SH, —NH₂, —CN, —CO₂H, —SO₂H, —SO₃H, —CONH₂, —OP(O)(OH)₂, —OPH(O)(OH), and oxo (═O), wherein each C₁-C₄ alkyl, phenyl or benzyl group is optionally substituted with one or more halo groups and/or one, two, three, four or five substituents independently selected from —OH, —SH, —NH₂, —CN, —CO₂H, —SO₂H, —SOH, —CONH₂, —OP(O)(OH)₂, and —OPH(O)(OH);
and/or any —R^{c} and any —R^{d} attached to the same carbon atom, together with the carbon atom to which they are attached, form C═O or a C₄-C₇ cycloalkyl or 4- to 7-membered heterocyclic group, wherein the C₄-C₇ cycloalkyl or 4- to 7-membered heterocyclic group is optionally substituted with one or more halo groups and/or one, two, three, four or five substituents independently selected from C₁-C₄ alkyl, phenyl, benzyl, —O—(C₁-C₄ alkyl), —O—CO—(C₁-C₄ alkyl), —CO₂—(C₁-C₄ alkyl), —OH, —SH, —NH₂, —CN, —CO₂H, —SO₂H, —SOH, —CONH₂, —OP(O)(OH)₂, —OPH(O)(OH), and oxo (═O), wherein each C₁-C₄ alkyl, phenyl or benzyl group is optionally substituted with one or more halo groups and/or one, two, three, four or five substituents independently selected from —OH, —SH, —NH₂, —CN, —CO₂H, —SO₂H, —SOH, —CONH₂, —OP(O)(OH)₂, and —OPH(O)(OH);
any —(CR^{c}R^{d})— may be replaced by —O— or —NR^{z}—, such that each —O— and —NR^{z}— is directly attached to two —(CR^{c}R^{d})— groups;
each -R^{d} is independently hydrogen, hydroxy, halo, C₁-C₆ alkyl, phenyl, or benzyl, wherein each C₁-C₆ alkyl, phenyl or benzyl group is optionally substituted with one or more halo and/or —OH groups;
each -R^{x} is hydrogen or C₁-C₄ alkyl;
each -R^{xx} is hydrogen or C₁-C₄ alkyl;
each -R^{y} is hydrogen or C₁-C₄ alkyl;
each -R^{z} is hydrogen, C₁-C₄ alkyl, or —R^{a};
m is 1, 2, 3, 4, 5, 6, 7, 8, 9, 10, 11 or 12;
each n is independently 0, 1 or 2;
each p is 1, 2, 3, 4, 5 or 6;
q is 1, 2, 3, 4, 5 or 6; and
each r is 1, 2, 3, 4, 5 or 6;
or a pharmaceutically acceptable salt thereof.

In one embodiment of the first, second, third, fourth or fifth aspect of the invention, the aminosulphonic acid has the structure

R^{a}R^{b}N—(CR^{c}R^{d})ₘ—SO₃H (formula I)

wherein:
-R^{a} is independently hydrogen, —R^{aaa}, —CHO, —COR^{aaa}, —CO₂R^{aaa}, —SOR^{aaa}, —SONH₂, —SONHR^{aaa}, —SON(R^{aaa})₂, —SO₂H, —SO₂R^{aaa}, —SO₂NH₂, —SO₂NHR^{aaa}, —SO₂N(R^{aaa})₂, —SOH, —SO₃R^{aaa}, —CONH₂, —CONHR^{aaa}, —CON(R^{aaa})₂, —C(NH)NH₂, —C(NH)NHR^{aaa}, —C(NH)N(R^{aaa})₂, —C(NR^{aaa})NHR^{aaa}, —C(NR^{aaa})N(R^{aaa})₂, -R^{aa}-R^{aaa}, —R^{aa}—OR^{aaa}, —R^{aa}—SR^{aaa}, —R^{aa}—OH, —R^{aa}—SH, —R^{aa}—CHO, —R^{aa}—COR^{aaa}, —R^{aa}—CO₂H, —R^{aa}—CO₂R^{aaa}, —R^{aa}—OCOR^{aaa}, —R^{aa}—SOR^{aaa}, —R^{aa}—SONH₂, —R^{aa}—SONHR^{aaa}, —R^{aa}—SON(R^{aaa})₂, —R^{aa}—SO₂H, —R^{aa}—SO₂R^{aaa}, —R^{aa}—SO₂NH₂, —R^{aa}—SO₂NHR^{aaa}, —R^{aa}—SO₂N(R^{aaa})₂, —R^{aa}—SO₃H, —R^{aa}—SO₃R^{aaa}, —R^{aa}—NH₂, —R^{aa}—NHR^{aaa}, —R^{aa}—N(R^{aaa})₂, —R^{aa}—CONH₂, —R^{aa}—CONHR^{aaa}, —R^{aa}—CON(R^{aaa})₂, —R^{aa}—C(NH)NH₂, —R^{aa}—C(NH)NHR^{aaa}, —R^{aa}—C(NH)N(R^{aaa})₂, —R^{aa}—C(NR^{aaa})NHR^{aaa}, or —R^{aa}—C(NR^{aaa})N(R^{aaa})₂; —R^{aa}— is independently C₁-C₄ alkylene or C₁-C₄ haloalkylene;
each -R^{aaa} is independently a C₁-C₃₀ alkyl (preferably C₁-C₁₂ alkyl or C₁-C₆ alkyl), C₂-C₃₀ alkenyl (preferably C₂-C₁₂ alkenyl or C₂-C₆ alkenyl), C₂-C₃₀ alkynyl (preferably C₂-C₁₂ alkynyl or C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SO₃H, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups;
-R^{b} is hydrogen, —R^{bbb}, —CHO, —COR^{bbb}, —CO₂R^{bbb}, —SOR^{bbb}, —SONH₂, —SONHR^{bbb}, —SON(R^{bbb})₂, —SO₂H, —SO₂R^{bbb}, —SO₂NH₂, —SO₂NHR^{bbb}, —SO₂N(R^{bbb})₂, —SO₃H, —SO₃R^{bbb}, —CONH₂, —CONHR^{bbb}, —CON(R^{bbb})₂, —C(NH)NH₂, —C(NH)NHR^{bbb}, —C(NH)N(R^{bbb})₂, —C(NR^{bbb})NHR^{bbb}, —C(NR^{bbb})N(R^{bbb})₂, —R^{bb}—R^{bbb}, —R^{bb}—OR^{bbb}, —R^{bb}—SR^{bbb}, —R^{bb}—OH, —R^{bb}—SH, —R^{bb}—CHO, —R^{bb}—COR^{bbb}, —R^{bb}—CO₂H, —R^{bb}—CO₂R^{bbb}, —R^{bb}—OCOR^{bbb}, —R^{bb}—SOR^{bbb}, —R^{bb}—SONH₂, —R^{bb}—SONHR^{bbb}, —R^{bb}—SON(R^{bbb})₂, —R^{bb}—SO₂H, —R^{bb}—SO₂R^{bbb}, —R^{bb}—SO₂NH₂, —R^{bb}—SO₂NHR^{bbb}, —R^{bb}—SO₂N(R^{bbb})₂, —R^{bb}—SO₃H, —R^{bb}—SO₃R^{bbb}, —R^{bb}—NH₂, —R^{bb}—NHR^{bbb}, —R^{bb}—N(R^{bbb})₂, —R^{bb}—CONH₂, —R^{bb}—CONHR^{bbb}, —R^{bb}—CON(R^{bbb})₂, —R^{bb}—C(NH)NH₂, —R^{bb}—C(NH)NHR^{bbb}, —R^{bb}—C(NH)N(R^{bbb})₂, —R^{bb}—C(NR^{bbb})NHR^{bbb}, or —R^{bb}—C(NR^{bbb})N(R^{bbb})₂; —R^{bb}— is independently C₁-C₄ alkylene or C₁-C₄ haloalkylene;
each -R^{bbb} is independently a C₁-C₃₀ alkyl (preferably C₁-C₁₂ alkyl or C₁-C₆ alkyl), C₂-C₃₀ alkenyl (preferably C₂-C₁₂ alkenyl or C₂-C₆ alkenyl), C₂-C₃₀ alkynyl (preferably C₂-C₁₂ alkynyl or C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SO₃H, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups;
or —R^{a} and —R^{b} together with the nitrogen atom to which they are attached, form a 4- to 7-membered heterocyclic group, which is optionally substituted with (i) one or more halo groups, and/or (ii) one, two, three, four or five substituents independently selected from —OH, —SH, —NH₂, —CN, —CO₂H, —SO₂H, —SOH, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo groups and/or one, two, three, four or five substituents independently selected from —OH, —SH, —NH₂, —CN, —CO₂H, —SO₂H, —SOH, —CONH₂, —OP(O)(OH)₂, and —OPH(O)(OH);
each -R^{c} is independently hydrogen, halo, —R^{cc}—R^{ccc}, —R^{cc}—OR^{ccc}, —R^{cc}—SR^{ccc}, —R^{cc}—OH, —R^{cc}—SH, —R^{cc}—CHO, —R^{cc}—COR^{ccc}, —R^{cc}—CO₂H, —R^{cc}—CO₂R^{ccc}, —R^{cc}—OCOR^{ccc}, —R^{cc}—SOR^{ccc}, —R^{cc}—SONH₂, —R^{cc}—SONHR^{ccc}, —R^{cc}—SON(R^{ccc})₂, —R^{cc}—SO₂H, —R^{cc}—SO₂R^{ccc}, —R^{cc}—SO₂NH₂, —R^{cc}—SO₂NHR^{ccc}, —R^{c}—SO₂N(R^{ccc})₂, —R^{cc}—SOH, —R^{cc}—SO₃R^{ccc}, —R^{cc}—NH₂, —R^{cc}—NHR^{ccc}, —R^{cc}—N(R^{ccc})₂, —R^{cc}—CONH₂, —R^{cc}—CONHR^{ccc}, —R^{cc}—CON(R^{ccc})₂, —R^{cc}—C(NH)NH₂, —R^{cc}—C(NH)NHR^{ccc}, —R^{cc}—C(NH)N(R^{ccc})₂, —R^{cc}—C(NR^{ccc})NHR^{ccc}, or —R^{cc}—C(NR^{cc})N(R^{cc})₂;
each -R^{cc}- is independently a bond, C₁-C₆ alkylene, C₂-C₆ alkenylene, or —(CH₂)ₙ—(C₆H₄)—(CH₂)ₙ—, each of which is optionally substituted with one or more halo and/or —OH groups;
each -R^{ccc}- is independently a C₁-C₁₂ alkyl (preferably C₁-C₆ alkyl), C₂-C₁₂ alkenyl (preferably C₂-C₆ alkenyl), C₂-C₁₂ alkynyl (preferably C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SO₃H, —CONH₂, —OP(O)(OH)₂, —OPH(O)(OH), and oxo (═O), and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups; and/or —R^{b} and any —R^{c} together with the atoms to which they are attached, form a 4- to 7-membered heterocyclic group, each of which is optionally substituted with one or more halo groups and/or one, two, three, four or five substituents independently selected from C₁-C₄ alkyl, phenyl, benzyl, —O—(C₁-C₄ alkyl), —O—CO—(C₁-C₄ alkyl), —CO₂—(C₁-C₄ alkyl), —OH, —SH, —NH₂, —CN, —CO₂H, —SO₂H, —SOH, —CONH₂, —OP(O)(OH)₂, —OPH(O)(OH)₂, and oxo (═O), wherein each C₁-C₄ alkyl, phenyl or benzyl group is optionally substituted with one or more halo groups and/or one, two, three, four or five substituents independently selected from —OH, —SH, —NH₂, —CN, —CO₂H, —SO₂H, —SOH, —CONH₂, —OP(O)(OH)₂, and —OPH(O)(OH);
and/or any two —R^{c} together with the carbon atoms to which they are attached, form a C₄-C₇ cycloalkyl or 4- to 7-membered heterocyclic group, each of which is optionally substituted with one or more halo groups and/or one, two, three, four or five substituents independently selected from C₁-C₄ alkyl, phenyl, benzyl, —O—(C₁-C₄ alkyl), —O—CO—(C₁-C₄ alkyl), —CO₂—(C₁-C₄ alkyl), —OH, —SH, —NH₂, —CN, —CO₂H, —SO₂H, —SO₃H, —CONH₂, —OP(O)(OH)₂, —OPH(O)(OH), and oxo (═O), wherein each C₁-C₄ alkyl, phenyl or benzyl group is optionally substituted with one or more halo groups and/or one, two, three, four or five substituents independently selected from —OH, —SH, —NH₂, —CN, —CO₂H, —SO₂H, —SOH, —CONH₂, —OP(O)(OH)₂, and —OPH(O)(OH);
and/or any —R^{c} and any —R^{d} attached to the same carbon atom, together with the carbon atom to which they are attached, form C═O or a C₄-C₇ cycloalkyl or 4- to 7-membered heterocyclic group, wherein the C₄-C₇ cycloalkyl or 4- to 7-membered heterocyclic group is optionally substituted with one or more halo groups and/or one, two, three, four or five substituents independently selected from C₁-C₄ alkyl, phenyl, benzyl, —O—(C₁-C₄ alkyl), —O—CO—(C₁-C₄ alkyl), —CO₂—(C₁-C₄ alkyl), —OH, —SH, —NH₂, —CN, —CO₂H, —SO₂H, —SOH, —CONH₂, —OP(O)(OH)₂, —OPH(O)(OH), and oxo (═O), wherein each C₁-C₄ alkyl, phenyl or benzyl group is optionally substituted with one or more halo groups and/or one, two, three, four or five substituents independently selected from —OH, —SH, —NH₂, —CN, —CO₂H, —SO₂H, —SOH, —CONH₂, —OP(O)(OH)₂, and —OPH(O)(OH);
any —(CR^{c}R^{d})— may be replaced by —O— or —NR^{z}—, such that each —O— and —NR^{z}— is directly attached to two —(CR^{c}R^{d})— groups;
each -R^{d} is independently hydrogen, hydroxy, halo, C₁-C₆ alkyl, phenyl, or benzyl, wherein each C₁-C₆ alkyl, phenyl or benzyl group is optionally substituted with one or more halo and/or —OH groups;
each -R^{z} is hydrogen or C₁-C₄ alkyl;
m is 1, 2, 3, 4, 5, 6, 7, 8, 9, 10, 11 or 12; and
each n is independently 0, 1 or 2;
or a pharmaceutically acceptable salt thereof.

In one embodiment of the first, second, third, fourth or fifth aspect of the invention, the aminosulphonic acid has the structure

R^{a}R^{b}N—(CR^{c}R^{d})ₘ—SO₃H (formula I)

wherein:
-R^{a} is independently hydrogen, —R^{aaa}, —CHO, —COR^{aaa}, —CO₂R^{aaa}, -R^{aa}-R^{aaa}, —R^{aa}—OR^{aaa}, —R^{aa}—SR^{aaa}, —R^{aa}—OH, —R^{aa}—SH, —R^{aa}—CHO, —R^{aa}—COR^{aaa}, —R^{aa}—CO₂H, —R^{aa}—CO₂R^{aaa}, —R^{aa}—OCOR^{aaa}, —R^{aa}—NH₂, —R^{aa}—NHR^{aaa}, or —R^{aa}—N(R^{aaa})₂; —R^{aa}— is independently C₁-C₄ alkylene or C₁-C₄ haloalkylene;
each -R^{aaa} is independently a C₁-C₁₂ alkyl (preferably C₁-C₆ alkyl), C₂-C₁₂ alkenyl (preferably C₂-C₆ alkenyl), C₂-C₁₂ alkynyl (preferably C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SO₃H, and —CONH₂, and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups; —R^{b} is hydrogen, —R^{bbb}, —CHO, —COR^{bbb}, —CO₂R^{bbb}, —R^{bb}—R^{bbb}, —R^{bb}—OR^{bbb}, —R^{bb}—SR^{bbb}, —R^{bb}—OH, —R^{bb}—SH, —R^{bb}—CHO, —R^{bb}—COR^{bbb}, —R^{bb}—CO₂H, —R^{bb}—CO₂R^{bbb}, —R^{bb}—OCOR^{bbb}, —R^{bb}—NH₂, —R^{bb}—NHR^{bbb}, or —R^{bb}—N(R^{bbb})₂; —R^{bb}— is independently C₁-C₄ alkylene or C₁-C₄ haloalkylene;
each -R^{bbb} is independently a C₁-C₁₂ alkyl (preferably C₁-C₆ alkyl), C₂-C₁₂ alkenyl (preferably C₂-C₆ alkenyl), C₂-C₁₂ alkynyl (preferably C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SOH, and —CONH₂, and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups;
or —R^{a} and —R^{b} together with the nitrogen atom to which they are attached, form a 4- to 7-membered heterocyclic group, which is optionally substituted with (i) one or more halo groups, and/or (ii) one, two, three, four or five substituents independently selected from —OH, —SH, —NH₂, —CN, —CO₂H, —SO₂H, —SOH, and —CONH₂, and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo groups and/or one, two, three, four or five substituents independently selected from —OH, —SH, —NH₂, —CN, —CO₂H, —SO₂H, —SOH, and —CONH₂;
each -R^{c} is independently hydrogen, halo, —R^{cc}—R^{ccc}, —R^{cc}—OR^{ccc}, —R^{cc}—SR^{ccc}, —R^{cc}—OH, —R^{cc}—SH, —R^{cc}—CHO, —R^{cc}—COR^{cc}, —R^{cc}—CO₂H, —R^{cc}—CO₂R^{ccc}, —R^{cc}—OCOR^{ccc}, —R^{cc}—NH₂, —R^{cc}—NHR^{ccc}, or —R^{cc}—N(R^{ccc})₂;
each -R^{cc}- is independently a bond, C₁-C₆ alkylene, or C₂-C₆ alkenylene, each of which is optionally substituted with one or more halo and/or —OH groups;
each -R^{ccc}- is independently a C₁-C₁₂ alkyl (preferably C₁-C₆ alkyl), C₂-C₁₂ alkenyl (preferably C₂-C₆ alkenyl), C₂-C₁₂ alkynyl (preferably C₂-C₆ alkynyl), C₃-C₆ cycloalkyl, phenyl, 4- to 7-membered heterocyclic or 5- to 6-membered heteroaryl group, each of which is optionally substituted with (i) one or more halo groups, and/or (ii) one, two, three, four or five substituents independently selected from —OH, —SH, —NH₂, —CN, —CO₂H, —SO₂H, —SOH, and —CONH₂, and/or (iii) one, two, three, four or five substituents independently selected from C₁-C₄ alkyl, phenyl, benzyl, —O—(C₁-C₄ alkyl), —O—CO—(C₁-C₄ alkyl), and —CO₂—(C₁-C₄ alkyl), each of which is optionally substituted with one or more halo and/or —OH groups;
and/or any two —R^{c} together with the carbon atoms to which they are attached, form a C₄-C₇ cycloalkyl or 4- to 7-membered heterocyclic group, each of which is optionally substituted with one or more halo groups and/or one, two, three, four or five substituents independently selected from C₁-C₄ alkyl, phenyl, benzyl, —O—(C₁-C₄ alkyl), —O—CO—(C₁-C₄ alkyl), —CO₂—(C₁-C₄ alkyl), —OH, —SH, —NH₂, —CN, —CO₂H, —SO₂H, —SO₃H, and —CONH₂, wherein each C₁-C₄ alkyl, phenyl or benzyl group is optionally substituted with one or more halo groups and/or one, two, three, four or five substituents independently selected from —OH, —SH, —NH₂, —CN, —CO₂H, —SO₂H, —SO₃H, and —CONH₂;
any —(CR^{c}R^{d})— may be replaced by —O— or —NR^{z}—, such that each —O— and —NR^{z}— is directly attached to two —(CR^{c}R^{d})— groups;
each -R^{d} is independently hydrogen, hydroxy, halo, C₁-C₆ alkyl, phenyl, or benzyl, wherein each C₁-C₆ alkyl, phenyl or benzyl group is optionally substituted with one or more halo and/or —OH groups;
each -R^{z} is hydrogen or C₁-C₄ alkyl; and
m is 1, 2, 3, 4, 5, 6, 7, 8, 9, 10, 11 or 12;
or a pharmaceutically acceptable salt thereof.

In one embodiment of the first, second, third, fourth or fifth aspect of the invention, the aminosulphonic acid has the structure

R^{a}R^{b}N—(CR^{c}R^{d})ₘ—SO₃H (formula I)

wherein:
-R^{a} is hydrogen or a C₁-C₄ alkyl, C₃-C₆ cycloalkyl, 4- to 7-membered heterocyclic or 5- to 6-membered heteroaryl group, wherein the C₁-C₄ alkyl, C₃-C₆ cycloalkyl, heterocyclic or heteroaryl group is optionally substituted with one or more fluoro groups and/or one, two or three substituents independently selected from C₁-C₄ alkyl, hydroxy, SO₃H and CONH₂;
-R^{b} is hydrogen or a C₁-C₄ alkyl, C₃-C₆ cycloalkyl, 4- to 7-membered heterocyclic or 5- to 6-membered heteroaryl group, wherein the C₁-C₄ alkyl, C₃-C₆ cycloalkyl, heterocyclic or heteroaryl group is optionally substituted with one or more fluoro groups and/or one, two or three substituents independently selected from C₁-C₄ alkyl, hydroxy, SO₃H and CONH₂; or
-R^{a} and —R^{b} together with the nitrogen atom to which they are attached, form a 4- to 7-membered heterocyclic or 5- to 6-membered heteroaryl group, wherein the heterocyclic or heteroaryl group is optionally substituted with one or more fluoro groups and/or one, two or three substituents independently selected from C₁-C₄ alkyl, hydroxy, SO₃H and CONH₂, wherein each C₁-C₄ alkyl group is optionally substituted with one or more fluoro groups and/or one, two or three substituents independently selected from hydroxy, SO₃H and CONH₂;
each -R^{c} is independently selected from hydrogen, hydroxy and fluoro, and/or any two —R^{C} together with the carbon atoms to which they are attached, form a C₅-C₆ cycloalkyl or 4- to 7-membered heterocyclic group, wherein the C₅-C₆ cycloalkyl or heterocyclic group is optionally substituted with one or more fluoro groups and/or one, two or three substituents independently selected from C₁-C₄ alkyl, hydroxy, SO₃H and CONH₂;
each -R^{d} is independently selected from hydrogen and fluoro; and
m is 1, 2, 3 or 4.

When both —R^{a} and —R^{b} are hydrogen, then the aminosulphonic acid of formula (I) is an N-unsubstituted aminosulphonic acid. When one or both of —R^{a} and —R^{b} are other than hydrogen, then the aminosulphonic acid of formula (I) is an N-substituted aminosulphonic acid.

In one embodiment, —R^{a} and —R^{b} together with the nitrogen atom to which they are attached, form a morpholinyl or piperazinyl group, wherein the piperazinyl group is optionally substituted with C₁-C₄ alkyl optionally substituted with one, two or three substituents independently selected from hydroxy and SO₃H.

In another embodiment, —R^{a} is H and —R^{b} is cyclohexyl.

In another embodiment, —R^{a} is H or C₁-C₄ alkyl substituted with one, two or three hydroxy groups, and —R^{b} is C₁-C₄ alkyl substituted with one, two or three substituents independently selected from hydroxy and CONH₂.

In another embodiment, —R^{a} and —R^{b} are hydrogen.

In one embodiment, —R^{c} is hydrogen or hydroxy.

In one embodiment, —R^{d} is hydrogen.

In one embodiment, m is 2, 3, 4, 5, 6, 7, 8, 9 or 10. In another embodiment, m is 2, 3, 4, 5, 6, 7 or 8. In another embodiment, m is 2, 3, 4, 5 or 6. In another embodiment, m is 2, 3 or 4. In another embodiment, m is 2 or 3.

In one embodiment, the aminosulphonic acid of formula (I) does not comprise any —OP(O)(OH)₂ or —OPH(O)(OH) groups.

In one embodiment, the aminosulphonic acid of formula (I) has a molecular weight of 4,000 Da or less, 2,000 Da or less, or 1,000 Da or less, or 500 Da or less.

Suitable aminosulphonic acids are also taught by Grygorenko et al (Tetrahedron, 2018, vol 74, pages 1355-1421) and in WO 2015/073648, both of which are incorporated herein by reference in their entirety.

Aminosulphinic Acid Buffers

In one embodiment of the first, second, third, fourth or fifth aspect of the invention, the composition comprises an aminosulphinic acid. In one embodiment, the aminosulphinic acid is present at a concentration of from 1 to 100 mmoles/L, or at a concentration of from 1 to 12 mmoles/L, or at a concentration of from 3 to 8 mmoles/L, or at a concentration of about 5 mmoles/L.

In one embodiment of the first, second, third, fourth or fifth aspect of the invention, the aminosulphinic acid is an aminoalkylsulphinic acid, such as an amino(C₁-C₄ alkyl) sulphinic acid or an amino(C₁-C₄ hydroxyalkyl)sulphinic acid.

In one embodiment of the first, second, third, fourth or fifth aspect of the invention, the aminosulphinic acid is an N-substituted aminosulphinic acid. In one embodiment, the N-substituted aminosulphinic acid is (HOCH₂CH₂)₂N—CH₂CH₂—SO₂H.

In one embodiment of the first, second, third, fourth or fifth aspect of the invention, the aminosulphinic acid is an N-unsubstituted aminosulphinic acid. In one embodiment, the N-unsubstituted aminosulphinic acid is 2-aminoethane-1-sulphinic acid [H₂N—CH₂CH₂—SO₂H], 3-aminopropane-1-sulphinic acid [H₂N—CH₂CH₂CH₂—SO₂H], 3-amino-2-hydroxypropane-1-sulphinic acid [H₂N—CH₂—CH(OH)—CH₂—SO₂H], 4-aminobutane-1-sulphinic acid [H₂N—CH₂CH₂CH₂CH₂—SO₂H], or aminobenzenesulphinic acid (including 2-, 3- and 4-aminobenzenesulphinic acid). In one embodiment, the N-unsubstituted aminosulphinic acid is 2-aminoethane-1-sulphinic acid, or 2-aminoethane-1-sulphinic acid bound to hyaluronic acid, or 2-aminoethane-1-sulphinic acid bound to chitosan, or 2-aminoethane-1-sulphinic acid bound to carrageenan. In one embodiment, the N-unsubstituted aminosulphinic acid is 2-aminoethane-1-sulphinic acid.

In one embodiment of the first, second, third, fourth or fifth aspect of the invention, the aminosulphinic acid has the structure

R^{a}R^{b}N—(CR^{C}R^{d})ₘ—SO₂H (formula II)

wherein —R^{a}, —R^{b}, —R^{c}, —R^{d} and m are as defined in relation to formula (I) above. For the avoidance of doubt it is noted that the embodiments of —R^{a}, —R^{b}, —R^{c}, —R^{d} and m outlined in relation to formula (I) above, apply equally to —R^{a}, —R^{b}, —R^{c}, —R^{d} and m of formula (II).

In one embodiment, the aminosulphinic acid of formula (II) does not comprise any —OP(O)(OH)₂ or —OPH(O)(OH) groups.

In one embodiment, the aminosulphinic acid of formula (II) has a molecular weight of 4,000 Da or less, 2,000 Da or less, or 1,000 Da or less, or 500 Da or less.

Aminosulphinic acids have a chiral sulphur atom. The present invention encompasses racemic mixtures of aminosulphinic acids, and enantiomerically enriched and enantiomerically pure aminosulphinic acids.

Without wishing to be bound by theory, it is currently believed that an aminosulphinic acid may be oxidised in vivo to the corresponding aminosulphonic acid.

Phosphate Buffers

In one embodiment of the first, second, third, fourth or fifth aspect of the invention, the composition comprises a phosphate. In one embodiment, the phosphate is present at a concentration of from 1 to 500 mmoles/L, or at a concentration of from 1 to 200 mmoles/L, or at a concentration of from 1 to 100 mmoles/L, or at a concentration of from 1 to 50 mmoles/L, or at a concentration of from 1 to 12 mmoles/L.

In one embodiment of the first, second, third, fourth or fifth aspect of the invention, the phosphate is an inorganic phosphate, such as a sodium phosphate (such as NaH₂PO₄, or Na₂HPO₄), a potassium phosphate (such as KH₂PO₄, or K₂HPO₄), a calcium phosphate (such as Ca₃(PO₄)₂, CaHPO₄, Ca(H₂PO₄)₂, or Ca₂P₂O₇), a magnesium phosphate (such as Mg₃(PO₄)₂, MgHPO₄, or Mg(H₂PO₄)₂), a zinc phosphate (such as Zn₃(PO₄)₂, ZnHPO₄, or Zn(H₂PO₄)₂), an iron phosphate (such as Fe₃(PO₄)₂, FeHPO₄, Fe(H₂PO₄)₂, or FePO₄), or a combination thereof. In one embodiment of the first, second, third, fourth or fifth aspect of the invention, the phosphate is an inorganic phosphate selected from NaH₂PO₄, Na₂HPO₄, KH₂PO₄, K₂HPO₄, or a combination thereof.

In one embodiment of the first, second, third, fourth or fifth aspect of the invention, the phosphate is an organic phosphate, such as an alkyl dihydrogen phosphate (such as methyl dihydrogen phosphate, ethyl dihydrogen phosphate, propyl dihydrogen phosphate, or butyl dihydrogen phosphate).

In one embodiment of the first, second, third, fourth or fifth aspect of the invention, the phosphate is an organic phosphate, such as a dialkyl hydrogen phosphate (such as dimethyl hydrogen phosphate, diethyl hydrogen phosphate, dipropyl hydrogen phosphate, or dibutyl hydrogen phosphate).

In one embodiment of the first, second, third, fourth or fifth aspect of the invention, the phosphate is an organic phosphate, such as a tris(perfluoroalkyl) trifluoro phosphate (such as tris(pentafluoroethyl) trifluoro phosphate).

In one embodiment of the first, second, third, fourth or fifth aspect of the invention, the phosphate is an organic phosphate, such as an amino phosphate. In one embodiment, the amino phosphate is an amino(C₁-C₄ alkyl)phosphate or an amino(C₁-C₄ hydroxyalkyl) phosphate.

In one embodiment of the first, second, third, fourth or fifth aspect of the invention, the amino phosphate has the structure

R^{a}R^{b}N—(CR^{c}R^{d})ₘ—OP(O)(OH)₂ (formula III)

wherein —R^{a}, —R^{b}, —R^{c}, —R^{d} and m are as defined in relation to formula (I) above. For the avoidance of doubt it is noted that the embodiments of —R^{a}, —R^{b}, —R^{c}, —R^{d} and m outlined in relation to formula (I) above, apply equally to —R^{a}, —R^{b}, —R^{c}, —R^{d} and m of formula (III).

In one embodiment, the amino phosphate of formula (III) has a molecular weight of 4,000 Da or less, 2,000 Da or less, or 1,000 Da or less, or 500 Da or less.

In one embodiment of the first, second, third, fourth or fifth aspect of the invention, the composition does not comprise an inorganic phosphate. In one embodiment of the first, second, third, fourth or fifth aspect of the invention, the composition does not comprise a phosphate.

Phosphite Buffers

In one embodiment of the first, second, third, fourth or fifth aspect of the invention, the composition comprises a phosphite. In one embodiment, the phosphite is present at a concentration of from 1 to 500 mmoles/L, or at a concentration of from 1 to 200 mmoles/L, or at a concentration of from 1 to 100 mmoles/L, or at a concentration of from 1 to 50 mmoles/L, or at a concentration of from 1 to 12 mmoles/L. In one embodiment of the first, second, third aspect, fourth or fifth of the invention, the phosphite is an inorganic phosphite, such as a sodium phosphite (such as NaH₂PO₃, or Na₂HPO₃), a potassium phosphite (such as KH₂PO₃, or K₂HPO₃), a calcium phosphite (such as Ca₃(PO₃)₂, CaHPO₃, or Ca(H₂PO₃)₂), a magnesium phosphite (such as Mg₃(PO₃)₂, MgHPO₃, or Mg(H₂PO₃)₂), a zinc phosphite (such as Zn₃(PO₃)₂, ZnHPO₃, or Zn(H₂PO₃)₂), an iron phosphite (such as Fe₃(PO₃)₂, FeHPO₃, Fe(H₂PO₃)₂, or FePO₃), or a combination thereof.

In one embodiment of the first, second, third, fourth or fifth aspect of the invention, the phosphite is an organic phosphite, such as an alkyl dihydrogen phosphite (such as methyl dihydrogen phosphite, or ethyl dihydrogen phosphite).

In one embodiment of the first, second, third, fourth or fifth aspect of the invention, the phosphite is an organic phosphite, such as a dialkyl hydrogen phosphite (such as dimethyl hydrogen phosphite, or diethyl hydrogen phosphite).

In one embodiment of the first, second, third, fourth or fifth aspect of the invention, the phosphite is an organic phosphite, such as an amino phosphite. In one embodiment, the amino phosphite is an amino(C₁-C₄ alkyl)phosphite or an amino(C₁-C₄ hydroxyalkyl) phosphite.

In one embodiment of the first, second, third, fourth or fifth aspect of the invention, the amino phosphite has the structure

R^{a}R^{b}N—(CR^{c}R^{d})ₘ—OPH(O)(OH) (formula IV)

wherein —R^{a}, —R^{b}, —R^{C}, —R^{d} and m are as defined in relation to formula (I) above. For the avoidance of doubt it is noted that the embodiments of —R^{a}, —R^{b}, —R^{c}, —R^{d} and m outlined in relation to formula (I) above, apply equally to —R^{a}, —R^{b}, —R^{c}, —R^{d} and m of formula (IV).

In one embodiment, the amino phosphite of formula (IV) has a molecular weight of 4,000 Da or less, 2,000 Da or less, or 1,000 Da or less, or 500 Da or less.

In one embodiment of the first, second, third, fourth or fifth aspect of the invention, the composition does not comprise an inorganic phosphite. In one embodiment of the first, second, third, fourth or fifth aspect of the invention, the composition does not comprise a phosphite.

Heteroaryl Buffers

In one embodiment of the first, second, third, fourth or fifth aspect of the invention, the composition comprises a 5- or 6-membered heteroaryl comprising one, two or three nitrogen atoms, such as pyridine, pyridazine, pyrimidine, pyrazine, triazine, pyrrole, pyrazole, imidazole, oxazole, isoxazole, thiazole, isothiazole, triazole, oxadiazole or thiadiazole, each of which may optionally be substituted, for example, with one, two or three C₁-C₆ alkyl groups. In one embodiment, the 5- or 6-membered heteroaryl comprising one, two or three nitrogen atoms is present at a concentration of from 1 to 100 mmoles/L, or at a concentration of from 1 to 12 mmoles/L, or at a concentration of from 3 to 8 mmoles/L, or at a concentration of about 5 mmoles/L.

In one embodiment of the first, second, third, fourth or fifth aspect of the invention, the composition comprises imidazole. In one embodiment, the imidazole is present at a concentration of from 1 to 100 mmoles/L, or at a concentration of from 1 to 12 mmoles/L, or at a concentration of from 3 to 8 mmoles/L, or at a concentration of about 5 mmoles/L.

Phenolic Acid Buffers

In one embodiment of the first, second, third, fourth or fifth aspect of the invention, the composition comprises a phenolic acid, such as a hydroxybenzoic acid or a hydroxycinnamic acid. In one embodiment, the phenolic acid is present at a concentration of from 1 to 100 mmoles/L, or at a concentration of from 1 to 12 mmoles/L, or at a concentration of from 3 to 8 mmoles/L, or at a concentration of about 5 mmoles/L.

Amino Acid, Peptide and Peptide Equivalent Buffers

In one embodiment of the first, second, third, fourth or fifth aspect of the invention, the composition comprises an amino acid, a peptide or a peptide equivalent. In one embodiment, the amino acid, peptide or peptide equivalent is present at a concentration of from 1 to 100 mmoles/L, or at a concentration of from 1 to 12 mmoles/L, or at a concentration of from 3 to 8 mmoles/L, or at a concentration of about 5 mmoles/L.

In one embodiment of the first, second, third, fourth or fifth aspect of the invention, the amino acid is an α-amino acid. In one embodiment, the α-amino acid is a natural α-amino acid. In one embodiment, the α-amino acid is cysteine, glycine, proline, hydroxyproline, alanine, valine, leucine, isoleucine, methionine, phenylalanine, tyrosine, tryptophan, arginine, histidine, lysine, aspartic acid, glutamic acid, serine, threonine, asparagine, glutamine, or citrulline. In one embodiment, the α-amino acid is proline, hydroxyproline, cysteine, leucine, histidine, or citrulline.

In one embodiment of the first, second, third, fourth or fifth aspect of the invention, the amino acid is a β-amino acid. In one embodiment, the β-amino acid is 3-aminopropionic acid or 2-aminobenzoic acid (anthranilic acid).

In one embodiment of the first, second, third, fourth or fifth aspect of the invention, the amino acid is a γ- or δ-amino acid, such as 4-aminobutanoic acid or 5-aminopentanoic acid.

In one embodiment of the first, second, third, fourth or fifth aspect of the invention, the peptide is a dipeptide. In one embodiment, the dipeptide comprises or consists of α-, β-, γ- or δ-amino acids. In one embodiment, the dipeptide comprises or consists of α-, β- or γ-amino acids. In one embodiment, the dipeptide consists of α-, β- or γ-amino acids. In one embodiment, the dipeptide consists of α- or β-amino acids. In one embodiment, the dipeptide consists of α-amino acids. In one embodiment, the dipeptide is amphoteric. In one embodiment, the dipeptide is glycyl glycine, alanyl glutamine, β-alanyl histidine (carnosine), β-alanyl 3-methylhistidine (anserine), glycyl glutamine, or an N-substituted derivative thereof (such as an N-acetylated derivative).

In one embodiment of the first, second, third, fourth or fifth aspect of the invention, the peptide is a tripeptide. In one embodiment, the tripeptide comprises or consists of α-, β-, γ- or δ-amino acids. In one embodiment, the tripeptide comprises or consists of α-, β- or γ-amino acids. In one embodiment, the tripeptide consists of α-, β- or γ-amino acids. In one embodiment, the tripeptide consists of α- or β-amino acids. In one embodiment, the tripeptide consists of α-amino acids. In one embodiment, the tripeptide is amphoteric. In one embodiment, the tripeptide is glutathione, lysine-proline-valine (KPV), or an N-substituted derivative thereof (such as an N-acetylated derivative).

In one embodiment of the first, second, third, fourth or fifth aspect of the invention, the peptide equivalent is a dipeptide equivalent. In one embodiment, the dipeptide equivalent comprises or consists of α-amino acids, β-amino acids, γ-amino acids, amino sulphonic acids and/or amino sulphinic acids. In one embodiment, the dipeptide equivalent consists of α-amino acids, β-amino acids, γ-amino acids, amino sulphonic acids and/or amino sulphinic acids. In one embodiment, the dipeptide equivalent is amphoteric. In one embodiment, the dipeptide equivalent is tauryl taurine (taurine dimer), glutamyl taurine, aspartyl taurine, seryl taurine, or an N-substituted derivative thereof (such as an N-acetylated derivative). In one embodiment, the dipeptide equivalent comprises taurine and an α-, β- or γ-amino acid, for example, the dipeptide equivalent is glutamyl taurine, aspartyl taurine, seryl taurine, or an N-substituted derivative thereof (such as an N-acetylated derivative).

In one embodiment of the first, second, third, fourth or fifth aspect of the invention, the peptide equivalent is a tripeptide equivalent. In one embodiment, the tripeptide equivalent comprises or consists of α-amino acids, β-amino acids, γ-amino acids, amino sulphonic acids and/or amino sulphinic acids. In one embodiment, the tripeptide equivalent consists of α-amino acids, β-amino acids, γ-amino acids, amino sulphonic acids and/or amino sulphinic acids. In one embodiment, the tripeptide equivalent is amphoteric. In one embodiment, the tripeptide equivalent is taurine trimer or an N-substituted derivative thereof (such as an N-acetylated derivative).

In one embodiment of the first, second, third, fourth or fifth aspect of the invention, the peptide equivalent is a di-, tri- or tetra-peptide equivalent comprising or consisting of α-amino acids and taurine.

Suitable peptide equivalents are also taught by Grygorenko et al (Tetrahedron, 2018, vol 74, pages 1355-1421), Vertesaljai et al (J Org Chem, 2014, vol 79(6), pages 2688-2693), and Ienaga et al (Chem Pharm Bull, 1988, vol 36(1), pages 70-77), all of which are incorporated herein by reference in their entirety.

In one embodiment of the first, second, third, fourth or fifth aspect of the invention, the composition comprises a fluorinated amino acid, a fluorinated peptide or a fluorinated peptide equivalent.

Polymeric Buffers

In one embodiment of the first, second, third, fourth or fifth aspect of the invention, the composition comprises a polymeric buffer.

In one embodiment, the polymeric buffer is a polymer which is a buffer in its own right, such chitosan, a polycation (such as polyethylenimine or polylysine), haemoglobin, ovalbumin, or a derivative thereof.

In another embodiment, the polymeric buffer is a bound buffer, i.e. a polymer which has been modified with buffering groups, such as a dendrimer, peptide, hyaluronic acid, chitosan or carrageenan modified with an N-substituted aminosulphonic acid (such as BES), N-unsubstituted aminosulphonic acid (such as taurine), aminosulphinic acid, phosphate, phosphite, heteroaryl (such as imidazole), phenolic acid, amino acid (such as proline), peptide, peptide equivalent or urea.

In one embodiment, the buffering group(s) of the polymeric buffer are present at a concentration of from 1 to 500 mmoles/L, or at a concentration of from 1 to 200 mmoles/L, or at a concentration of from 1 to 100 mmoles/L, or at a concentration of from 1 to 50 mmoles/L, or at a concentration of from 1 to 12 mmoles/L.

In one embodiment of the first, second, third, fourth or fifth aspect of the invention, the N-substituted aminosulphonic acid (such as BES), N-unsubstituted aminosulphonic acid (such as taurine), aminosulphinic acid, phosphate, phosphite, heteroaryl (such as imidazole), phenolic acid, amino acid (such as proline), peptide, peptide equivalent or urea is bound to a polymer such as hyaluronic acid or chitosan. Typically in such an embodiment, the polymer to which the N-substituted aminosulphonic acid, N-unsubstituted aminosulphonic acid, aminosulphinic acid, phosphate, phosphite, heteroaryl, phenolic acid, amino acid, peptide, peptide equivalent or urea is bound is dissolved or suspended in water. In one embodiment, the polymer is water-soluble chitosan. In one embodiment, the polymer is chitosan coupled with urocanic acid. In one embodiment, the polymer is water-soluble chitosan coupled with urocanic acid.

In one embodiment of the first, second, third, fourth or fifth aspect of the invention, the composition comprises a carrageenan derivative or analogue as buffer. Carrageenan and derivatives and analogues thereof and their preparation are disclosed, for example, by Yamada et al, Carbohydrate Polymers, 1997, vol 32, pages 51-55, which is incorporated herein by reference in its entirety.

In one embodiment of the first, second, third, fourth or fifth aspect of the invention, the composition comprises a dendrimer as buffer.

Dendrimers are highly ordered, highly branched, polymeric molecules. Dendrimers are defined by three components: a central core, an interior dendritic structure (the branches), and an exterior surface with functional surface groups. Typically, dendrimers are symmetric about the core, and often adopt a spherical three-dimensional morphology. Their branched architecture gives rise to a large number of surface sites relative to the total molecular volume.

Poly(amidoamine), or PAMAM, is a class of dendrimer which is made of repetitively branched subunits of amide and amine functionality. Like other dendrimers, PAMAMs have a sphere-like shape overall and an internal molecular architecture consisting of tree-like branching, with each outward ‘layer’, or generation, containing exponentially more branching points.

PAMAMs can be synthesised by using ethylene diamine as a core initiator and accomplishing the outward growth by alternating between the following two reactions:
1. Michael addition of the amino-terminated surface onto methyl acrylate, resulting in an ester-terminated outer layer, and
2. Coupling with ethylene diamine to achieve a new amino-terminated surface.

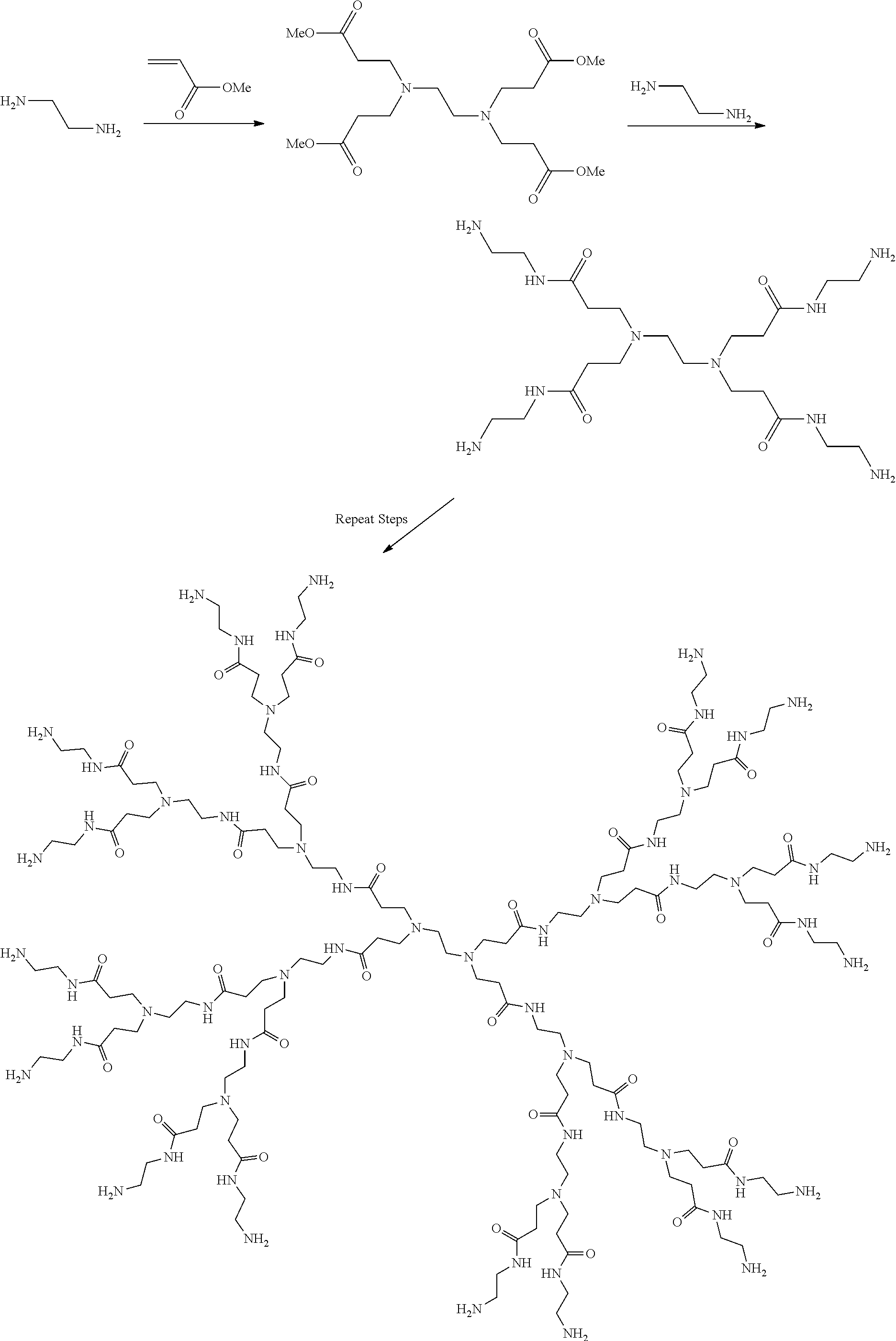

Conventional PAMAMs can be converted into buffers, for example, by hydrolysing surface esters to the corresponding carboxylic acids (see (a) in Scheme below) or by end capping surface amines with acids such as carboxylic acids (see (b) in Scheme below) or sulphonic acids (see (c) and (d) in Scheme below). The resulting PAMAM dendrimer, having an amine rich basic core and an acid terminated surface, is an efficient buffer.

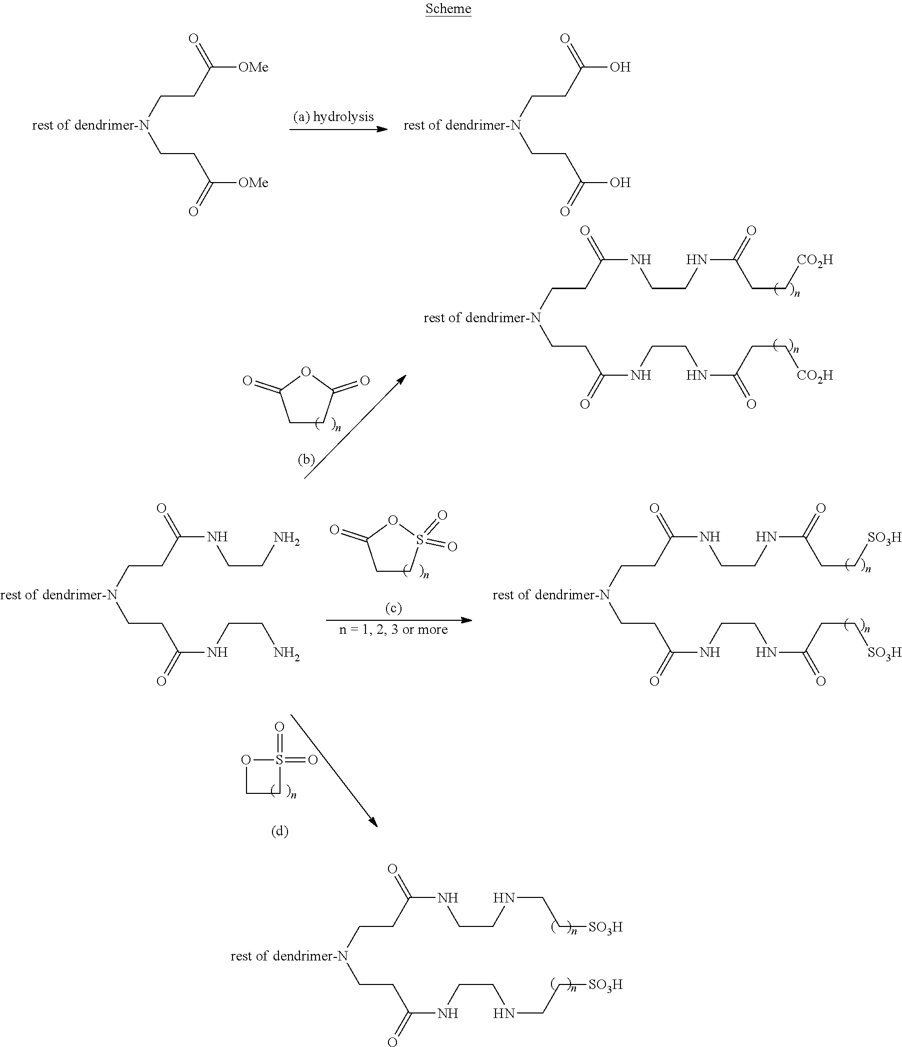

Suitable dendrimers, which can be surface modified for example with carboxylic or sulphonic acids as described above, are also taught by Dykes, J Chem Technol Biotechnol, 2001, vol 76, pages 903-918; Abbasi et al, Nanoscale Research Letters, 2014, vol 9, article 247; Baig et al, IJAPBC, 2015, vol 4(1), pages 44-59; Shahi et al, Int J Pharm Sci Rev Res, 2015, vol 33(1), pages 187-198; and Gupta et al, J Appl Pharm Sci, 2015, vol 5(3), pages 117-122; all of which are incorporated herein by reference in their entirety.

Urea Buffers

In one embodiment of the first, second, third, fourth or fifth aspect of the invention, the composition comprises a urea R₂N—CO—NR₂, wherein each R is independently selected from hydrogen or C₁-C₆ alkyl. In one embodiment, the urea is present at a concentration of from 1 to 100 mmoles/L, or at a concentration of from 1 to 12 mmoles/L, or at a concentration of from 3 to 8 mmoles/L, or at a concentration of about 5 mmoles/L.

In one embodiment of the first, second, third, fourth or fifth aspect of the invention, the composition comprises urea (H₂N—CO—NH₂). In one embodiment, the urea is present at a concentration of from 1 to 100 mmoles/L, or at a concentration of from 1 to 12 mmoles/L, or at a concentration of from 3 to 8 mmoles/L, or at a concentration of about 5 mmoles/L.

Ionic Liquid Buffers

In one embodiment of the first, second, third, fourth or fifth aspect of the invention, the composition comprises an ionic liquid buffer. Ionic liquid buffers are made up of anions and cations. In one embodiment, the buffer composition comprises an ionic liquid buffer comprising (i) an anion derived from a Good's buffer (such as those listed in FIG. 1), an α-amino acid (such as a natural α-amino acid such as those described above including proline, hydroxyproline, cysteine, leucine, histidine and citrulline), a β-amino acid (such as those described above), an aminosulphonic acid (such as those described above including BES and taurine), or an aminosulphinic acid (such as those described above), and (ii) a cation selected from a tetraalkyl ammonium (such as cholinium, [Me₂N(CH₂CH₂OH)₂]+, tetramethyl ammonium, tetraethyl ammonium or tetrabutyl ammonium), dialkyl imidazolium (such as 1-ethyl-3-methyl-imidazolium or 1-butyl-3-methyl-imidazolium), alkyl pyridinium (such as methyl pyridinium), alkyl pyrrolidinium (such as N-butyl-N-methyl-pyrrolidinium), or tetraalkyl phosphonium (such as tetrabutyl phosphonium or tributyl methyl phosphonium) cation. In one embodiment, the buffer composition comprises an ionic liquid buffer comprising (i) an anion derived from a Good's buffer (in particular Tricine, TES, CHES, HEPES, BES or MES), and (ii) a cholinium cation. Alternatively, the anion could be FeCl₄⁻, NH₂SO₃⁻, bis(trifluoromethylsulfonyl)imide, a halide (such as chloride, bromide, or iodide), a carboxylate (such as acetate, methoxyacetate, trifluoroacetate, trichloroacetate, oxalate, propionate, malonate, lactate, pyruvate, pivalate, benzoate, salicylate, caprylate, caprynate, palmitate, stearate, or heptafluorobutonate), a sulfate (such as an alkyl sulfate (such as methylsulfate or ethylsulfate)), a sulfonate (such as an alkylsulfonate (such as methanesulfonate, trifluoromethanesulfonate, or hydroxymethanesulfonate)), a phosphate (such as dihydrogen phosphate, an alkyl hydrogen phosphate (such as methyl hydrogen phosphate, ethyl hydrogen phosphate, propyl hydrogen phosphate, or butyl hydrogen phosphate), a dialkyl phosphate (such as dimethyl phosphate, diethyl phosphate, dipropyl phosphate, or dibutyl phosphate), or a tris(perfluoroalkyl) trifluoro phosphate (such as tris(pentafluoroethyl) trifluoro phosphate)), or a phosphite (such as an alkyl hydrogen phosphite (such as methyl hydrogen phosphite, or ethyl hydrogen phosphite), or a dialkyl phosphite (such as dimethyl phosphite, or diethyl phosphite)). The cation could alternatively be an ammonium (such as [Me₂NH(CH₂CH₂OH)]+), a pentaalkyl guanidinium (such as N,N,N′,N′,N″-pentamethyl-guanidinium), a dialkyl morpholinium (such as ethyl-methyl-morpholinium), or an alkyl pyrimidinium (such as methyl-pyrimidinium) cation.

In one embodiment of the first or third aspect of the invention, the ionic liquid buffer is used in an aqueous composition, such as an aqueous solution. In another embodiment of the first or third aspect of the invention, the ionic liquid buffer is used in a non-aqueous medium. In another embodiment of the first or third aspect of the invention, the ionic liquid buffer itself is used as medium.

In one embodiment of the second, fourth or fifth aspect of the invention, the ionic liquid buffer is used in an aqueous solution.

Ionic liquid buffers can be prepared as described by MacFarlane et al (Chemical Communications, 2010, vol 46, pages 7703-7705), Matias et al (RSC Advances, 2014, vol 4, 15597-15601), Taha et al (Green Chemistry, 2014, vol 16(6), pages 3149-3159) or Taha et al (Chemistry, 2015, vol 21(12), pages 4781-4788), all of which are incorporated herein in their entirety by reference. For example, choline hydroxide can be reacted with a Good's buffer to prepare an ionic liquid buffer.

Buffer Amounts

In one embodiment of the first, second, third, fourth or fifth aspect of the invention, the Good's buffer, N-substituted aminosulphonic acid (such as BES), N-unsubstituted aminosulphonic acid (such as taurine), aminosulphinic acid, phosphate, phosphite, heteroaryl (such as imidazole), phenolic acid, amino acid (such as proline), peptide, peptide equivalent, urea, or buffering group(s) of the polymeric buffer is present in the composition of the present invention at a concentration of from 1 to 500 mmoles/L, or at a concentration of from 1 to 200 mmoles/L, or at a concentration of from 1 to 100 mmoles/L, or at a concentration of from 1 to 50 mmoles/L, or at a concentration of from 1 to 12 mmoles/L.

In one embodiment of the first, second, third, fourth or fifth aspect of the invention, if more than one of these buffers is used in the composition of the present invention, i.e. if more than one of a Good's buffer, aminosulphonic acid, aminosulphinic acid, phosphate, phosphite, heteroaryl, phenolic acid, amino acid, peptide, peptide equivalent, urea, or polymeric buffer is used, then the total concentration of buffers or buffering groups present in the composition is from 1 to 500 mmoles/L, or from 1 to 200 mmoles/L, or from 1 to 100 mmoles/L, or from 1 to 50 mmoles/L, or from 1 to 12 mmoles/L.

In one embodiment of the first, second, third, fourth or fifth aspect of the invention, at least 700 μl 1M aqueous HCl (typically at least 1000 μl 1M aqueous HCl, more typically at least 1300 μl 1M aqueous HCl) are required to change the pH of 100 mL of the composition at 20° C. by 1 unit.

In another embodiment of the first, second, third, fourth or fifth aspect of the invention, at least 200 μl of 1M aqueous NaOH (typically at least 250 μl of 1M aqueous NaOH, more typically at least 300 μl of 1M aqueous NaOH) are required to change the pH of 100 mL of the composition at 20° C. by 1 unit.

In one embodiment of the first, second, third, fourth or fifth aspect of the invention, the composition of the present invention has a buffer capacity at 20° C. of at least 0.002, preferably at least 0.01, preferably at least 0.02.

In one embodiment of the first, second, third, fourth or fifth aspect of the invention, the buffer used in the composition of the present invention is pharmaceutically acceptable.

In one embodiment, the buffer used in the composition of the present invention is on the FDA (US Food and Drug Administration) GRAS (generally recognized as safe) list.

In a preferred embodiment of the first, second, third, fourth or fifth aspect of the invention, the composition is an aqueous composition comprising;
(i) one or more components selected from BES, imidazole and taurine;
(ii) hydrogen carbonate ions or an equivalent thereof; and
(iii) optionally an α- or β-amino acid (such as a natural α-amino acid such as proline, hydroxyproline, cysteine, leucine, histidine or citrulline).

In a first specific preferred embodiment of the first, second, third, fourth or fifth aspect of the invention, the composition is an aqueous composition comprising (i) BES; and (ii) hydrogen carbonate ions or an equivalent thereof. Preferably the composition is an aqueous composition comprising (i) BES and (ii) hydrogen carbonate ions. Preferably the aqueous composition further comprises zinc ions.

In a second specific preferred embodiment of the first, second, third, fourth or fifth aspect of the invention, the composition is an aqueous composition comprising (i) imidazole; and (ii) hydrogen carbonate ions or an equivalent thereof. Preferably the composition is an aqueous composition comprising (i) imidazole and (ii) hydrogen carbonate ions. Preferably the aqueous composition further comprises zinc ions.

In a third specific preferred embodiment of the first, second, third, fourth or fifth aspect of the invention, the composition is an aqueous composition comprising (i) imidazole; (ii) an α- or β-amino acid (such as a natural α-amino acid such as proline); and (iii) hydrogen carbonate ions or an equivalent thereof. Preferably the composition is an aqueous composition comprising (i) imidazole, (ii) an α- or β-amino acid (such as a natural α-amino acid such as proline) and (iii) hydrogen carbonate ions. Preferably the aqueous composition further comprises zinc ions.

In a fourth specific preferred embodiment of the first, second, third, fourth or fifth aspect of the invention, the composition is an aqueous composition comprising (i) imidazole; (ii) taurine; and (iii) hydrogen carbonate ions or an equivalent thereof.

Preferably the composition is an aqueous composition comprising (i) imidazole, (ii) taurine and (iii) hydrogen carbonate ions. Preferably the aqueous composition further comprises zinc ions.

In a fifth specific preferred embodiment of the first, second, third, fourth or fifth aspect of the invention, the composition is an aqueous composition comprising (i) taurine; and (ii) hydrogen carbonate ions or an equivalent thereof. Preferably the composition is an aqueous composition comprising (i) taurine and (ii) hydrogen carbonate ions. Preferably the aqueous composition further comprises zinc ions.

In a sixth specific preferred embodiment of the first, second, third, fourth or fifth aspect of the invention, the composition is an aqueous composition comprising (i) taurine; (ii) an α- or β-amino acid (such as a natural α-amino acid such as proline); and (iii) hydrogen carbonate ions or an equivalent thereof. Preferably the composition is an aqueous composition comprising (i) taurine, (ii) an α- or β-amino acid (such as a natural α-amino acid such as proline) and (iii) hydrogen carbonate ions. Preferably the aqueous composition further comprises zinc ions.

In a seventh specific preferred embodiment of the first, second, third, fourth or fifth aspect of the invention, the composition is an aqueous composition comprising (i) imidazole; (ii) taurine; (iii) an α- or β-amino acid (such as a natural α-amino acid such as proline); and (iv) hydrogen carbonate ions or an equivalent thereof. Preferably the composition is an aqueous composition comprising (i) imidazole, (ii) taurine, (iii) an α- or β-amino acid (such as a natural α-amino acid such as proline) and (iv) hydrogen carbonate ions. Preferably the aqueous composition further comprises zinc ions.

In one embodiment of the first, second, third, fourth or fifth aspect of the invention, the composition comprises calcium ions present at a concentration of from 0.1 to 2.5 mmoles/L, or at a concentration of from 1 to 2.5 mmoles/L, or at a concentration of from 1.1 to 1.4 mmoles/L, or at a concentration of about 1.25 mmoles/L. In one embodiment, at least some of the calcium ions are provided by dissolving calcium chloride, calcium sulphate, calcium acetate or calcium ascorbate in water. In one embodiment, at least some of the calcium ions are provided by dissolving calcium chloride in water. In another embodiment, substantially all of the calcium ions are provided by dissolving calcium chloride in water.

In one embodiment of the first, second, third, fourth or fifth aspect of the invention, the composition comprises magnesium ions present at a concentration of from 0.1 to 2.5 mmoles/L, or at a concentration of from 0.2 to 0.6 mmoles/L, or at a concentration of from 0.3 to 0.5 mmoles/L, or at a concentration of about 0.45 mmoles/L. In one embodiment, at least some of the magnesium ions are provided by dissolving magnesium chloride, magnesium sulphate, magnesium acetate or magnesium ascorbate in water. In one embodiment, at least some of the magnesium ions are provided by dissolving magnesium chloride in water. In another embodiment, substantially all of the magnesium ions are provided by dissolving magnesium chloride in water.

In one embodiment of the first, second, third, fourth or fifth aspect of the invention, the calcium ions and magnesium ions are present at a molar concentration ratio of from 5:1 to 1:1 (calcium ions: magnesium ions), or at a molar concentration ratio of from 4:1 to 2:1, or at a molar concentration ratio of about 3:1.

In one embodiment of the first, second, third, fourth or fifth aspect of the invention, the calcium ions are present at a concentration of from 1 to 2.5 mmoles/L, and the magnesium ions are present at a concentration of from 0.2 to 0.6 mmoles/L. In another embodiment, the calcium ions are present at a concentration of from 1.1 to 1.4 mmoles/L, and the magnesium ions are present at a concentration of from 0.3 to 0.5 mmoles/L.

In one embodiment of the first, second, third, fourth or fifth aspect of the invention, the composition comprises hydrogen carbonate ions present at a concentration of from 1 to mmoles/L, or at a concentration of from 10 to 40 mmoles/L, or at a concentration of from 21 to 35 mmoles/L, or at a concentration of from 22 to 29 mmoles/L, or at a concentration of about 25 mmoles/L. The range of from 1 to less than 21 mmoles/L and the range of from more than 35 to 45 mmoles/L are less preferred ranges, but still within the scope of the present invention. In one embodiment, at least some of the hydrogen carbonate ions are provided by dissolving sodium hydrogen carbonate, potassium hydrogen carbonate, sodium carbonate or potassium carbonate in water. In one embodiment, at least some of the hydrogen carbonate ions are provided by dissolving sodium hydrogen carbonate in water. In another embodiment, substantially all of the hydrogen carbonate ions are provided by dissolving sodium hydrogen carbonate in water.

In one embodiment of the first, second, third, fourth or fifth aspect of the invention, the composition comprises an equivalent of hydrogen carbonate ions. In one embodiment, the equivalent of hydrogen carbonate ions is a compound capable of being converted (e.g. hydrolysed) into hydrogen carbonate ions. In one embodiment, the equivalent of hydrogen carbonate ions is acetate ions. Without wishing to be bound by theory, it is currently believed that acetate ions convert into hydrogen carbonate ions in vivo. In one embodiment, the acetate ions are present at a concentration of from 1 to 45 mmoles/L, or at a concentration of from 10 to 40 mmoles/L, or at a concentration of from 21 to 35 mmoles/L, or at a concentration of from 22 to 29 mmoles/L, or at a concentration of about 25 mmoles/L. The range of from 1 to less than 21 mmoles/L and the range of from more than 35 to 45 mmoles/L are less preferred ranges, but still within the scope of the present invention. In one embodiment, at least some of the acetate ions are provided by dissolving sodium acetate, zinc acetate, potassium acetate, calcium acetate, magnesium acetate or ferrous acetate in water. In one embodiment, at least some of the acetate ions are provided by dissolving sodium acetate or zinc acetate in water. In one embodiment, at least some of the acetate ions are provided by dissolving sodium acetate in water. In another embodiment, substantially all of the acetate ions are provided by dissolving sodium acetate in water.

In one embodiment of the first, second, third, fourth or fifth aspect of the invention, the composition comprises hydrogen carbonate ions and an equivalent of hydrogen carbonate ions, such as acetate ions. In one embodiment, the hydrogen carbonate ions and acetate ions together are present at a concentration of from 1 to 45 mmoles/L, or at a concentration of from 10 to 40 mmoles/L, or at a concentration of from 21 to 35 mmoles/L, or at a concentration of from 22 to 29 mmoles/L, or at a concentration of about 25 mmoles/L. The range of from 1 to less than 21 mmoles/L and the range of from more than 35 to 45 mmoles/L are less preferred ranges, but still within the scope of the present invention.

In one embodiment of the first, second, third, fourth or fifth aspect of the invention, the composition comprises potassium ions present at a concentration of from 2.5 to 200 mmoles/L, or at a concentration of from 2.5 to 100 mmoles/L, or at a concentration of from 2.5 to 50 mmoles/L, or at a concentration of from 2.5 to 20 mmoles/L, or at a concentration of from 2.5 to 6.2 mmoles/L, or at a concentration of from 3 to 6 mmoles/L, or at a concentration of about 5 mmoles/L. In one embodiment, at least some of the potassium ions are provided by dissolving potassium chloride, potassium sulphate, potassium acetate, potassium ascorbate, potassium hydrogen carbonate or potassium carbonate in water. In one embodiment, at least some of the potassium ions are provided by dissolving potassium chloride, potassium sulphate or potassium acetate in water. In one embodiment, at least some of the potassium ions are provided by dissolving potassium chloride in water. In another embodiment, substantially all of the potassium ions are provided by dissolving potassium chloride in water.

In one embodiment of the first, second, third, fourth or fifth aspect of the invention, the composition comprises chloride ions present at a concentration of from 96 to 126 mmoles/L, or at a concentration of from 100 to 122 mmoles/L, or at a concentration of about 118 mmoles/L. In one embodiment, at least some of the chloride ions are provided by dissolving calcium chloride, magnesium chloride, potassium chloride, sodium chloride, zinc chloride, ferrous chloride, ferric chloride, choline chloride, thiamine pyrophosphate chloride and/or carnitine chloride in water. In one embodiment, at least some of the chloride ions are provided by dissolving calcium chloride, magnesium chloride, potassium chloride, sodium chloride, zinc chloride, choline chloride, thiamine pyrophosphate chloride and/or carnitine chloride in water.

In one embodiment of the first, second, third, fourth or fifth aspect of the invention, the composition comprises sodium ions present at a concentration of from 100 to 400 mmoles/L, or at a concentration of from 100 to 300 mmoles/L, or at a concentration of from 100 to 200 mmoles/L, or at a concentration of from 100 to 150 mmoles/L, or at a concentration of from 115 to 140 mmoles/L, or at a concentration of about 135 mmoles/L. In one embodiment, at least some of the sodium ions are provided by dissolving sodium hydrogen carbonate, sodium carbonate, sodium chloride, sodium sulphate, sodium acetate or sodium ascorbate in water. In one embodiment, at least some of the sodium ions are provided by dissolving sodium hydrogen carbonate, sodium chloride, sodium sulphate or sodium acetate in water. In one embodiment, at least some of the sodium ions are provided by dissolving sodium hydrogen carbonate and/or sodium chloride in water. In another embodiment, substantially all of the sodium ions are provided by dissolving sodium hydrogen carbonate and/or sodium chloride in water.

In one embodiment of the first, second, third, fourth or fifth aspect of the invention, the composition comprises zinc ions present at a concentration of from 0.1 to 200 μmoles/L, or at a concentration of from 0.1 to 150 μmoles/L, or at a concentration of from 0.1 to 100 μmoles/L, or at a concentration of from 0.1 to 50 moles/L, or at a concentration of from 1 to 10 μmoles/L. In one embodiment, at least some of the zinc ions are provided by dissolving zinc chloride, zinc sulphate, zinc acetate, a zinc phosphate (such as Zn₃(PO₄)₂, ZnHPO₄, or Zn(H₂PO₄)₂), a zinc phosphite (such as Zn₃(PO₃)₂, ZnHPO₃, or Zn(H₂PO₃)₂), zinc ascorbate, zinc gluconate, zinc oxide, zinc pivolate, zinc picolinate or zinc pyrithione in water. In one embodiment, at least some of the zinc ions are provided by dissolving zinc chloride in water. In another embodiment, substantially all of the zinc ions are provided by dissolving zinc chloride in water. In one embodiment, at least some of the zinc ions are provided together with thymulin or in the form of zinc thymulin complex. In one embodiment, substantially all of the zinc ions are provided together with thymulin or in the form of zinc thymulin complex.

Intracellular zinc ion (Zn²⁺) concentration can be increased with zinc-ionophores such as ascorbate ions, calcimycin, chloroquine, hydroxychloroquine, clioquinol, diiodohydroxyquinoline, dithiocarbamates (such as pyrrolidine dithiocarbamate), epigallocatechin gallate, hinokitiol, PBT2, pyrithione, quercetin, or zincophorin.

Accordingly, in one embodiment of the first, second, third, fourth or fifth aspect of the invention, the composition comprises zinc ions and further comprises a zinc-ionophore. In one embodiment of the first, second, third, fourth or fifth aspect of the invention, the composition comprises zinc ions and further comprises ascorbate ions, calcimycin, chloroquine, hydroxychloroquine, clioquinol, diiodohydroxyquinoline, a dithiocarbamate (such as pyrrolidine dithiocarbamate), epigallocatechin gallate, hinokitiol, PBT2, pyrithione, quercetin, or zincophorin. In one embodiment of the first, second, third, fourth or fifth aspect of the invention, the composition comprises zinc ions and further comprises pyrithione. In one embodiment of the first, second, third, fourth or fifth aspect of the invention, the composition comprises zinc pyrithione. In one embodiment of the first, second, third, fourth or fifth aspect of the invention, the composition comprises zinc ascorbate.

In one embodiment of the first, second, third, fourth or fifth aspect of the invention, the composition comprises transferrin present at a concentration of from 1 to 100 μmoles/L, or at a concentration of from 1 to 50 μmoles/L, or at a concentration of from 1 to 25 μmoles/L. The transferrin may be used in the form of holo-transferrin, apotransferrin or mono ferric transferrin.

In one embodiment of the first, second, third, fourth or fifth aspect of the invention, the composition comprises iron ions present at a concentration of from 1 to 100 μmoles/L, or at a concentration of from 10 to 100 moles/L. The iron ions may be used in the form of Fe²⁺ or Fe³⁺. In one embodiment, at least some of the iron ions are provided by dissolving ferrous fumarate, ferrous gluconate, ferrous succinate, ferrous sulphate, ferric ammonium citrate, ferrous bisglycinate, ferric nitrate, ferrous acetate, ferrous ascorbate, ferrous chloride or ferric chloride in water.

In one embodiment of the first, second, third, fourth or fifth aspect of the invention, the composition is an aqueous composition further comprising one or more of:

| | | |
|---|---|---|
| (a) | from 2 to 11 mmoles/L | glucose; |
| (b) | from 50 to 150 umoles/L | glycerol; |
| (c) | from 7 to 15 μmoles/L | choline ions; |
| (d) | from 5 to 400 μmoles/L | glutamate; |
| (e) | from 5 to 200 μmoles/L | aspartate; |
| (f) | from 100 to 2000 μmoles/L | glutamine; |
| (g) | from 20 to 215 μmoles/L | pyroglutamate; |
| (h) | from 20 to 200 μmoles/L | arginine; |
| (i) | from 1 to 250 nmoles/L | thiamine pyrophosphate ions; |
| (j) | from 40 to 100 μmoles/L | carnitine; |
| (k) | from 5 to 600 mIU/L | porcine or human insulin; |
| (l) | from 20 to 200 μmoles/L | hyaluronic acid; |
| (m) | from 1 to 100 μmoles/L | transferrin; |
| (n) | from 20 to 250 μmoles/L | leucine; |
| (o) | from 10 to 100 μmoles/L | linoleic acid; |
| (p) | from 200 to 1000 μmoles/L | cholesterol; |
| (q) | from 20 to 500 μmoles/L | pyridoxal-5-phosphate; or |
| (r) | from 10 to 250 μmoles/L | chitosan. |

In one embodiment, if a composition of the first, second, third, fourth or fifth aspect of the invention comprises glucose, it may be used in the form of D-glucose. In one embodiment, if a composition of the first, second, third, fourth or fifth aspect comprises glutamate, it may be used in the form of L-glutamate. In one embodiment, if a composition of the first, second, third, fourth or fifth aspect comprises aspartate, it may be used in the form of L-aspartate. In one embodiment, if a composition of the first, second, third, fourth or fifth aspect comprises glutamine, it may be used in the form of L-glutamine. In one embodiment, if a composition of the first, second, third, fourth or fifth aspect comprises carnitine, it may be used in the form of L-carnitine. In one embodiment, if a composition of the first, second, third, fourth or fifth aspect comprises leucine, it may be used in the form of L-leucine.

In one embodiment, if a composition of the first, second, third, fourth or fifth aspect of the invention comprises insulin, this may be human insulin, such as human recombinant insulin.

In one embodiment, if a composition of the first, second, third, fourth or fifth aspect of the invention comprises choline ions, thiamine pyrophosphate ions and/or carnitine, these may be independently provided to the composition as chloride, sulphate, acetate or other salts. In one embodiment, a chloride salt is used.

In one embodiment of the first, second, third, fourth or fifth aspect of the invention, the composition comprises one or more of hyaluronic acid, transferrin, leucine, linoleic acid, cholesterol, or pyridoxal-5-phosphate. It is currently believed that these components may preserve cell membrane functions.

In one embodiment of the first, second, third, fourth or fifth aspect of the invention, the composition comprises chitosan. Chitosan can act as an antifungal and antibacterial agent. Chitosan can also act as a buffer, a thickening or gelling agent, or a toll-like receptor modulator.

In one embodiment of the first, second, third, fourth or fifth aspect of the invention, the composition is an aqueous composition comprising:

| | | |
|---|---|---|
| (a) | from 1 to 12 mmoles/L | N-substituted aminosulphonic acid |
| | | (preferably BES); and |
| (b) | from 21 to 35 mmoles/L | hydrogen carbonate ions. |

In this embodiment, the composition may optionally further comprise one or more additional components such as those selected from zinc ions, calcium ions, magnesium ions, potassium ions, chloride ions, sodium ions, glucose (preferably D-glucose), glycerol, choline ions, glutamate (preferably L-glutamate), aspartate (preferably L-aspartate), glutamine (preferably L-glutamine), carnitine (preferably L-carnitine), thiamine pyrophosphate ions, and porcine or human insulin (preferably recombinant human insulin).

In another embodiment of the first, second, third, fourth or fifth aspect of the invention, the composition is an aqueous composition comprising:

| | | |
|---|---|---|
| (a) | from 1 to 12 mmoles/L | N-substituted aminosulphonic acid |
| (b) | from 21 to 35 mmoles/L | (preferably BES); |
| (c) | from 0.1 to 200 μmoles/L | hydrogen carbonate ions; and |
| | | zinc ions. |

In this embodiment, the composition may optionally further comprise one or more additional components such as those selected from calcium ions, magnesium ions, potassium ions, chloride ions, sodium ions, glucose (preferably D-glucose), glycerol, choline ions, glutamate (preferably L-glutamate), aspartate (preferably L-aspartate), glutamine (preferably L-glutamine), carnitine (preferably L-carnitine), thiamine pyrophosphate ions, and porcine or human insulin (preferably recombinant human insulin).

In another embodiment of the first, second, third, fourth or fifth aspect of the invention, the composition is an aqueous composition comprising:

| | | |
|---|---|---|
| (a) | from 1 to 12 mmoles/L | N-substituted aminosulphonic acid |
| | | (preferably BES); |
| (b) | from 0.1 to 2.5 mmoles/L | calcium ions; |
| (c) | from 0.02 to 2.5 mmoles/L | magnesium ions; |
| (d) | from 21 to 35 mmoles/L | hydrogen carbonate ions; |
| (e) | from 2.5 to 6.2 mmoles/L | potassium ions; |
| (f) | from 96 to 126 mmoles/L | chloride ions; |
| (g) | from 100 to 150 mmoles/L | sodium ions; and |
| (h) | optionally from 0.1 to 200 μmoles/L | zinc ions. |

In this embodiment, the composition may optionally further comprise one or more additional components such as those selected from glucose (preferably D-glucose), glycerol, choline ions, glutamate (preferably L-glutamate), aspartate (preferably L-aspartate), glutamine (preferably L-glutamine), carnitine (preferably L-carnitine), thiamine pyrophosphate ions, and porcine or human insulin (preferably recombinant human insulin).

In another embodiment of the first, second, third, fourth or fifth aspect of the invention, the composition is an aqueous composition comprising:

| | | |
|---|---|---|
| (a) | from 1 to 12 mmoles/L | N-substituted aminosulphonic acid |
| | | (preferably BES); |
| (b) | from 0.1 to 2.5 mmoles/L | calcium ions; |
| (c) | from 0.02 to 2.5 mmoles/L | magnesium ions; |
| (d) | from 21 to 35 mmoles/L | hydrogen carbonate ions; |
| (e) | from 2.5 to 6.2 mmoles/L | potassium ions; |
| (f) | from 96 to 126 mmoles/L | chloride ions; |
| (g) | from 100 to 150 mmoles/L | sodium ions; |
| (h) | from 2 to 11 mmoles/L | glucose (preferably D-glucose); |
| (i) | from 50 to 150 μmoles/L | glycerol; |
| (j) | from 7 to 15 μmoles/L | choline ions; |
| (k) | from 5 to 400 μmoles/L | glutamate (preferably L-glutamate); |
| (l) | from 5 to 200 μmoles/L | aspartate (preferably L-aspartate); |
| (m) | from 100 to 2000 μmoles/L | glutamine (preferably L-glutamine); |
| (n) | from 40 to 100 μmoles/L | carnitine (preferably L-carnitine); |
| (o) | optionally from 1 to 250 nmoles/L | thiamine pyrophosphate ions; |
| (p) | optionally from 5 to 600 mIU/L | porcine or human insulin (preferably |
| | | recombinant human insulin); and |
| (q) | optionally from 0.1 to 200 μmoles/L | zinc ions. |

In another embodiment of the first, second, third, fourth or fifth aspect of the invention, the composition is an aqueous composition comprising:

| | | |
|---|---|---|
| (a) | about 5 mmoles/L | N-substituted aminosulphonic acid |
| | | (preferably BES); |
| (b) | about 1.25 mmoles/L | calcium ions; |
| (c) | about 0.45 mmoles/L | magnesium ions; |
| (d) | about 25 mmoles/L | hydrogen carbonate ions; |
| (e) | about 5 mmoles/L | potassium ions; |
| (f) | about 118-119 mmoles/L | chloride ions; |
| (g) | about 135 mmoles/L | sodium ions; |
| (h) | about 10 mmoles/L | D-glucose; |
| (i) | about 110 μmoles/L | glycerol; |
| (j) | about 10 μmoles/L | choline ions; |
| (k) | about 300 μmoles/L | L-glutamate; |
| (l) | about 20 μmoles/L | L-aspartate; |
| (m) | about 400 μmoles/L | L-glutamine; |
| (n) | about 50 μmoles/L | L-carnitine; |
| (o) | optionally about 40 nmoles/L | thiamine pyrophosphate ions; |
| (p) | optionally about 28 mIU/L | recombinant human insulin; |
| (q) | optionally about 50 μmoles/L | zinc ions (alternatively about 10 μmoles/L |
| | | zinc ions); |
| (r) | optionally about 50 μmoles/L | transferrin (alternatively about 25 |
| | | μmoles/L transferrin); |
| (s) | optionally about 120 μmoles/L | L-leucine; |
| (t) | optionally about 70 μmoles/L | linoleic acid; |
| (u) | optionally about 400 μmoles/L | cholesterol; and |
| (v) | optionally about 100 μmoles/L | pyridoxal-5-phosphate. |

In another embodiment of the first, second, third, fourth or fifth aspect of the invention, the composition is an aqueous composition comprising:

| | | |
|---|---|---|
| (i) | from 1 to 500 mmoles/L | buffer (preferably phosphate buffer, |
| | | such as a combination of KH₂PO₄ and |
| | | Na₂HPO₄); and |
| (ii) | from 0.1 to 200 g/L | essential oil with antiviral activity |
| | | (preferably clove oil, eucalyptus oil, |
| | | basil oil, ginger oil, an extract or |
| | | component of any of these, or a |
| | | combination thereof). |

In this embodiment, the composition may optionally further comprise one or more additional components such as those selected from zinc ions (preferably ZnCl₂), NaCl, KCl, MgCl₂, NaHCO₃, xylitol, EDTA, CaCl₂, glycerol, HPMC, PEG 400, poloxamer 188, benzalkonium chloride and sodium hyaluronate.

In yet another embodiment of the first, second, third, fourth or fifth aspect of the invention, the composition is an aqueous composition comprising:

| | | |
|---|---|---|
| (i) | from 1 to 500 mmoles/L | buffer (preferably phosphate buffer, such as a |
| | | combination of KH₂PO₄ and Na₂HPO₄); |
| (ii) | from 0.1 to 200 μmoles/L | zinc ions (preferably ZnCl₂); |
| (iii) | from 0.1 to 50 g/L | clove oil or an extract or component thereof; |
| (iv) | from 0.1 to 50 g/L | eucalyptus oil or an extract or component thereof; |
| (v) | from 0.1 to 50 g/L | basil oil or an extract or component thereof; and |
| (vi) | from 0.1 to 50 g/L | ginger oil or an extract or component thereof. |

In this embodiment, the composition may optionally further comprise one or more additional components such as those selected from NaCl, KCl, MgCl₂, NaHCO₃, xylitol, EDTA, CaCl₂, glycerol, HPMC, PEG 400, poloxamer 188, benzalkonium chloride and sodium hyaluronate.

In yet another embodiment of the first, second, third, fourth or fifth aspect of the invention, the composition is an aqueous composition comprising:

| | | |
|---|---|---|
| (i) | from 25 to 250 mmoles/L | phosphate buffer, such as a combination of |
| | | KH₂PO₄ and Na₂HPO₄; |
| (ii) | from 0.1 to 50 μmoles/L | zinc ions (preferably ZnCl₂); |
| (iii) | from 21 to 35 mmoles/L | hydrogen carbonate ions; |
| (iv) | from 0.1 to 10 g/L | clove oil or an extract or component thereof; |
| (v) | from 0.1 to 10 g/L | eucalyptus oil or an extract or component thereof; |
| (vi) | from 0.1 to 10 g/L | basil oil or an extract or component thereof; and |
| (vii) | from 0.1 to 10 g/L | ginger oil or an extract or component thereof. |

In this embodiment, the composition may optionally further comprise one or more additional components such as those selected from NaCl, KCl, MgCl₂, xylitol, EDTA, CaCl₂, glycerol, HPMC, PEG 400, poloxamer 188, benzalkonium chloride and sodium hyaluronate.

In one embodiment of the first, second, third, fourth or fifth aspect of the invention, the composition further comprises an antioxidant. In one embodiment, the antioxidant is vitamin E or a derivative thereof (such as α-tocopherol polyethylene glycol 1000 succinate (TPGS), or α-tocopherol succinate), ascorbic acid, or sodium metabisulfite.

In one embodiment of the first, second, third, fourth or fifth aspect of the invention, the composition further comprises an emulsifying agent. Preferably the emulsifying agent is pharmaceutically acceptable or acceptable for use in food (see, for example, Encyclopedia for Food and Health, 2016, Elsevier, ScienceDirect, eds. Caballero, Finglas and Toldra, which is incorporated herein by reference in its entirety). Typically, emulsifying agents are prepared by alcoholysis or direct esterification of fatty acids (such as acetic, citric, lactic, tartaric, monoacetyl tartaric, diacetyl tartaric, stearic or linoleic acid) with polyols (such as glycerol, propylene glycol or sorbitol). Further processing by reaction with ethylene oxide or esterification with organic acids produces a wide range of emulsifiers with differing properties. Typical emulsifying agents are lecithin; mono- and diglycerides of fatty acids and their esters; polyglycerol fatty acid esters; polyoxyethylene sorbitan fatty acid esters (polysorbates); propylene glycol fatty acid esters; sorbitan fatty acid esters (Span's); stearoyl lactylates; and sucrose esters.

In one embodiment of the first, second, third, fourth or fifth aspect of the invention, the composition further comprises an active agent. In one embodiment, the active agent is an antibiotic, an antiviral agent, a nitric oxide producing agent, an immunoglobulin, a toll-like receptor modulator, a proton pump inhibitor, a lipid, an immunostimulant, an anti-inflammatory agent, an antiseptic, or an antifungal agent.

In one embodiment of the first, second, third, fourth or fifth aspect of the invention, the composition further comprises an antibiotic. In one embodiment, the antibiotic is chloramphenicol, streptomycin, penicillin, nanomycopulitin, a macrolide antibiotic (such as azithromycin, erythromycin, clarithromycin, roxithromycin, fidaxomicin or telithromycin), or Triticum repens. In one embodiment, the antibiotic is present at a concentration of from 10 to 150 mg/L, or at a concentration of from 60 to 120 mg/L, or at a concentration of about 100 mg/L.

In one embodiment of the first, second, third, fourth or fifth aspect of the invention, the composition further comprises an antiviral agent. In one embodiment, the antiviral agent is chlorpheniramine, carbinoxamine, oseltamivir, favipiravir, remdesivir, ribavirin, ritonavir, lopinavir, darunavir, D-xylose, a dendrimer (such as SPL7013), a peptidic viral fusion inhibitor, a peptidase inhibitor (such as a cathepsin inhibitor), a protease inhibitor, a helicase inhibitor, an antibody (such as a polyclonal or monoclonal antibody, such as an anti-CD3 monoclonal antibody, such as foralumab), a nanobody (such as a nanobody that impedes spike-ACE2 interactions, for example Nb6 or mNb6-tri as disclosed in https://doi.org/10.1101/2020.08.08.238469 which is incorporated herein by reference in its entirety), a sulfated polysaccharide [such as carrageenan (such as kappa-carrageenan, iota-carrageenan, lambda-carrageenan, or Carragelose®), fucoidan, sulfated fucan, sulfated galactan, or a sulfated glycosaminoglycan (such as heparin, 6-O-desulfated heparin, enoxaparin, or 6-O-desulfated enoxaparin)], ivermectin, grapefruit seed extract, chloroquine or a derivative or analogue thereof (such as hydroxychloroquine, ferroquine, desethylamodiaquine or pyrimethamine), quinine or a salt, derivative or analogue thereof (such as quinidine or mefloquine), pyronaridine, an alcohol (such as ethanol or isopropanol), an arylsulphonic acid or a diazenylarylsulphonic acid (such as described in Becht et al, J Gen Vir, 1968, vol 2(2), pages 261-268; Akerfeldt et al, J Med Chem, 1971, vol 14(7), pages 596-600; and Hoffmann et al, Front Microbiol, 2017, vol 8, article 205; all of which are incorporated herein by reference in their entirety), povidone-iodine, hydrogen peroxide, a surfactant, glycyrrhizin, 18β-glycyrrhetinic acid, licorice extract, reynoudiol, Reynoutria japonica extract, Eriobotrya japonica extract, pyrazofurin, a cyclodextrin (such as a cyclodextrin modified with mercaptoundecane sulfonic acids), a terpene (such as beta-caryophyllene, eucalyptol or citral), cannabidiol, oxymetazoline, xylometazoline, interferon, or a mixture thereof. In one embodiment, the antiviral agent is a fatty acid [such as a saturated fatty acid (e.g. caprylic, capric, lauric, myristic, palmitic or stearic acid), an omega-3 unsaturated fatty acid (e.g. α-linolenic, eicosapentaenoic or docosahexaenoic acid), an omega-6 unsaturated fatty acid (e.g. linoleic, γ-linolenic or arachidonic acid), an omega-7 unsaturated fatty acid (e.g. palmitoleic or heptadecenoic acid), or an omega-9 unsaturated fatty acid (e.g. oleic or erucic acid)], or a derivative thereof (such as an ester, amide, carbamate or a fluorinated derivative), or a mixture thereof; in particular, in one embodiment, the antiviral agent is linoleic acid or a derivative thereof (such as an ester, amide, carbamate or a fluorinated derivative). Fatty acids are lipophilic and can be formulated as micelles in the compositions of the present invention. In one embodiment, the antiviral agent is a griffithsin polypeptide or an analogue thereof (such as Q-griffithsin). In one embodiment, the antiviral agent is a polypeptide comprising SEQ ID NO: 1 (which is the same as SEQ ID NO: 3 disclosed in WO 2007/064844 which is incorporated herein by reference in its entirety) or a fragment thereof comprising at least eight contiguous amino acids, a nucleic acid encoding the polypeptide, or an antibody to the polypeptide. In a preferred embodiment, the antiviral agent is an essential oil with antiviral activity, such as clove oil (Syzygium aromaticum), basil oil (Ocimum basilicum), ginger oil (Zingiber officinale), eucalyptus oil (Eucalyptus globulus), saffron oil (Crocus sativus), cannabis oil (Cannabis sativa), tea tree oil (Melaleuca alternifolia), black seed oil (Nigella sativa), dill oil, dill seed oil, nutmeg oil, cinnamon oil, bay leaf oil, garlic oil, or geranium oil. In another preferred embodiment, the antiviral agent is the antiviral agent which imparts antiviral activity to an essential oil, such as eugenol, zingiberene, eucalyptol, jensenone, ursolic acid, caryophyllene, estragole, 1,8-cineole, camphor, thymol, eugenol epoxide, apigenin, linalool, rosmarinic acid, 6-gingerol, 6-shogaol, gingeronone A, crocetin, crocin, picrocrocin, safranal, caflanone, tetrahydrocannabinol, cannabidiol (or its metabolite 7-hydroxycannabidiol), cannabinol, tetrahydrocannabivarin, terpinen-4-ol, terpinolene, α-terpineol, thymoquinone, ar-curcumene, β-sesquiphellandrene, α-farnesene, α-phellandrene, β-phellandrene, camphene, β-bisabolene, α-terpinene, γ-terpinene, β-cymene, α-pinene, or a mono- or polyunsaturated fatty acid such as palmitoleic acid (16:1 n-7), heptadecenoic acid (17:1 n-7), oleic acid (18:1 n-9), linoleic acid (18:2 n-6), α-linolenic acid (18:3 n-3), γ-linolenic acid (18:3 n-6), arachidonic acid (20:4 n-6), or erucic acid (22:1 n-9). In one embodiment of the first, second, third, fourth or fifth aspect of the invention, the composition is substantially free of antiviral agent other than an essential oil or an extract or component thereof. In one embodiment, the composition is substantially free of antiviral agent other than clove oil, eucalyptus oil, basil oil, ginger oil, an extract or component of any of these, or a combination thereof.

In one embodiment of the first, second, third, fourth or fifth aspect of the invention, where the composition comprises clove oil, basil oil, ginger oil, eucalyptus oil, saffron oil, cannabis oil, tea tree oil, black seed oil, dill oil, dill seed oil, nutmeg oil, cinnamon oil, bay leaf oil, garlic oil, geranium oil, or a combination thereof, the total concentration of these oils in the composition is from 0.1 to 100 g/L, or from 0.1 to 50 g/L, or from 0.1 to 40 g/L, or from 0.1 to 25 g/L, or from 0.1 to 20 g/L, or from 0.5 to 40 g/L, or from 0.5 to 25 g/L, or from 1 to 20 g/L.

In one embodiment of the first, second, third, fourth or fifth aspect of the invention, the composition further comprises a nitric oxide (NO) producing agent. In one embodiment, the nitric oxide producing agent is S-nitroso-N-acetyl-penicillamine, arginine, sodium pyruvate, serine, cysteine, lysine, quinine or a salt, derivative or analogue thereof (such as quinidine or mefloquine), chloroquine or a derivative or analogue thereof (such as hydroxychloroquine, ferroquine, desethylamodiaquine or pyrimethamine), a denatonium salt such as denatonium benzoate, absinthin, salicin, phenylthiocarbamide, a homoserine lactone, a sesquiterpene lactone, sodium thiocyanate, linoleic acid or a derivative thereof (such as an ester, amide, carbamate or a fluorinated derivative), or 6-n-propylthiouracil.

Naturally occurring nitric oxide in the nasal cavities is a primary defense in humans. Nitric oxide is needed to kill invading viruses and prevents/reduces the rate and severity of viral infections with and viral replication of viruses such as rhinoviruses, influenza viruses and coronaviruses. Therefore, in one embodiment of the first, second, third, fourth or fifth aspect of the invention, the composition further comprises a nitric oxide donor such as S-nitroso-N-acetyl-penicillamine. Nitric oxide is produced in vivo by the action of nitric oxide synthase which catalyses nitric oxide production from L-arginine. Therefore, in one embodiment of the first, second, third, fourth or fifth aspect of the invention, the composition further comprises a compound which stimulates or allows the nasal mucosa to generate nitric oxide such as arginine (typically L-arginine typically in an amount of about 40-140 μmol/L), sodium pyruvate (typically in an amount of about 10-115 μmol/L), serine (typically L-serine typically in an amount of about 25-160 μmol/L), cysteine (typically L-cysteine typically in an amount of about 1-110 μmol/L) or lysine (typically L-lysine typically in an amount of about 45-205 μmol/L). Bitter taste receptor agonists may stimulate bitter taste receptors on nasal epithelial cells to stimulate nitric oxide production. Therefore, in one embodiment of the first, second, third, fourth or fifth aspect of the invention, the composition further comprises a bitter taste receptor agonist such as quinine or a salt, derivative or analogue thereof (such as quinidine or mefloquine), chloroquine or a derivative or analogue thereof (such as hydroxychloroquine, ferroquine, desethylamodiaquine or pyrimethamine), a denatonium salt such as denatonium benzoate, absinthin, salicin, phenylthiocarbamide, a homoserine lactone (such as C4HSL, C6HSL or C12HSL), a sesquiterpene lactone, sodium thiocyanate, or 6-n-propylthiouracil.

In one embodiment of the first, second, third, fourth or fifth aspect of the invention, the composition further comprises an immunoglobulin. In one embodiment, the immunoglobulin is immunoglobulin IgY, such as chicken immunoglobulin IgY. In one embodiment, the immunoglobulin is virus-specific immunoglobulin. In one embodiment, the immunoglobulin is virus-specific immunoglobulin IgY, such as virus-specific chicken immunoglobulin IgY.

In one embodiment of the first, second, third, fourth or fifth aspect of the invention, the composition further comprises a toll-like receptor modulator (such as a TLR2, TLR3 or TLR4 modulator). In one embodiment, the toll-like receptor modulator is chitosan, hyaluronic acid, a hyaluronic acid degradation product, carrageenan (such as lambda-carrageenan), chloroquine or a derivative or analogue thereof (such as hydroxychloroquine, ferroquine, desethylamodiaquine or pyrimethamine), quinine or a salt, derivative or analogue thereof (such as quinidine or mefloquine), Pam2Cys or a derivative or analogue thereof, Pam3Cys or a derivative or analogue thereof, or a mixture thereof. Pam2Cys and Pam3Cys and their derivatives and analogues are highly lipophilic and can be formulated as micelles in the compositions of the present invention. In one embodiment, the toll-like receptor modulator is a TLR3 modulator such as polyinosinic acid and/or polycytidylic acid and/or their derivatives, for example as disclosed in WO 2018/091965 which is incorporated herein by reference in its entirety.

In one embodiment of the first, second, third, fourth or fifth aspect of the invention, the composition further comprises a proton pump inhibitor. In one embodiment, the proton pump inhibitor is omeprazole, esomeprazole, lansoprazole, dexlansoprazole, pantoprazole, rabeprazole or ilaprazole.

In one embodiment of the first, second, third, fourth or fifth aspect of the invention, the composition further comprises a lipid. In one embodiment, the lipid is sphingosine or an analogue thereof.

In one embodiment of the first, second, third, fourth or fifth aspect of the invention, the composition further comprises an immunostimulant. In one embodiment, the immunostimulant is Allium sativum or Echinacea purpurea.

In one embodiment of the first, second, third, fourth or fifth aspect of the invention, the composition further comprises an anti-inflammatory agent. In one embodiment, the anti-inflammatory agent is Matricaria chamomilla or viola tricolor.

In one embodiment of the first, second, third, fourth or fifth aspect of the invention, the composition further comprises an antiseptic. In one embodiment, the antiseptic is cetylpyridinium chloride.

In one embodiment of the first, second, third, fourth or fifth aspect of the invention, the composition further comprises an antifungal agent. In one embodiment, the antifungal agent is chitosan, posaconazole, nanomycopulitin or amphotericin B.

In one embodiment of the first, second, third, fourth or fifth aspect of the invention, the composition further comprises a thickening or gelling agent. In one embodiment, the thickening or gelling agent is selected from polyethylene glycol (such as PEG400), microcrystalline cellulose, carboxymethyl cellulose, carboxymethyl cellulose sodium, hydroxypropyl methyl cellulose, monoglycerides, diglycerides, locust bean gum, polyvinylpyrrolidone, alginate, chitosan, agarose, gellan gum, and carrageenan (such as kappa-carrageenan, iota-carrageenan, lambda-carrageenan, or Carragelose®). Without wishing to be bound by theory, it is currently believed that a thickening or gelling agent may aid the adherence of the composition to the subject's cell membranes during use.

In one embodiment of the first, second, third, fourth or fifth aspect of the invention, the composition is substantially free of carbomer. In one embodiment, the composition is substantially free of carrageenan. In one embodiment, the composition is substantially free of thickening or gelling agent.

In one embodiment of the first, second, third, fourth or fifth aspect of the invention, the composition is substantially free of citrate buffer. In one embodiment of the first, second, third, fourth or fifth aspect of the invention, the composition is substantially free of lactate buffer. In one embodiment of the first, second, third, fourth or fifth aspect of the invention, the composition is substantially free of phosphate buffer. In one embodiment of the first, second, third, fourth or fifth aspect of the invention, the composition is substantially free of borate buffer. In one embodiment of the first, second, third, fourth or fifth aspect of the invention, the composition is substantially free of glycine buffer. In one embodiment of the first, second, third, fourth or fifth aspect of the invention, the composition is substantially free of citrate, lactate, phosphate, phosphite, borate and glycine buffer. In one embodiment of the first, second, third, fourth or fifth aspect of the invention, the composition is substantially free of any buffer other than a Good's buffer. In one embodiment of the first, second, third, fourth or fifth aspect of the invention, the composition is substantially free of any buffer other than an N-substituted aminosulphonic acid buffer. In one embodiment of the first, second, third, fourth or fifth aspect of the invention, the composition is substantially free of any buffer other than an N-substituted aminosulphonic acid Good's buffer.

In one embodiment of the first, second, third, fourth or fifth aspect of the invention, the composition is substantially free of serum and/or serum extract.

In one embodiment of the first, second, third, fourth or fifth aspect of the invention, the composition is substantially free of oncotic agent. In one embodiment of the first, second, third, fourth or fifth aspect of the invention, the composition is substantially free of plasma expander. In one embodiment of the first, second, third, fourth or fifth aspect of the invention, the composition is substantially free of dextran. In one embodiment of the first, second, third, fourth or fifth aspect of the invention, the composition is substantially free of pyruvate. In one embodiment of the first, second, third, fourth or fifth aspect of the invention, the composition is substantially free of phosphonoacetic acid.

In one embodiment of the first, second, third, fourth or fifth aspect of the invention, the Good's buffer, aminosulphonic acid, aminosulphinic acid, phosphate, phosphite, heteroaryl, phenolic acid, amino acid, peptide, peptide equivalent, polymeric buffer, urea or ionic liquid buffer has a pKₐ value in aqueous solution of from 6.7 to 7.9 at a temperature of 37° C., or from 6.7 to 7.7 at a temperature of 37° C., or from 7.1 to 7.5 at a temperature of 37° C. In one embodiment of the first, second, third, fourth or fifth aspect of the invention, the N-substituted aminosulphonic acid has a pKₐ value in aqueous solution of from 7.1 to 7.5 at 37° C.

In one embodiment of the first, second, third, fourth or fifth aspect of the invention, the composition has a pH of from 6.7 to 7.9 at a temperature of 37° C. In one embodiment of the first, second, third, fourth or fifth aspect of the invention, the composition has a pH of from 6.7 to 7.7 at a temperature of 37° C. In one embodiment of the first, second, third, fourth or fifth aspect of the invention, the composition has a pH of from 7.0 to 7.5 at a temperature of 37° C.

In one embodiment of the first, second, third, fourth or fifth aspect of the invention, the composition is non-colloidal. In one embodiment of the first, second, third, fourth or fifth aspect of the invention, the composition has a viscosity at 20° C. of 0.1-99 centipoise, typically 0.1-90 centipoise, typically 0.2-80 centipoise, typically 0.2-70 centipoise, typically 0.3-60 centipoise, typically 0.3-50 centipoise, typically 0.4-40 centipoise, typically 0.4-30 centipoise, typically 0.5-20 centipoise, typically 0.5-10 centipoise, typically 0.9-3.1 centipoise. In one embodiment of the first, second, third, fourth or fifth aspect of the invention, the composition has a viscosity at 20° C. of from more than 3.1 to 99 centipoise, typically from more than 3.1 to 90 centipoise, typically from more than 3.1 to 80 centipoise, typically from more than 3.1 to 70 centipoise, typically from more than 3.1 to 60 centipoise, typically from more than 3.1 to 50 centipoise, typically from more than 3.1 to 40 centipoise, typically from more than 3.1 to 30 centipoise, typically from more than 3.1 to 20 centipoise, typically from more than 3.1 to 10 centipoise. In one embodiment of the first, second, third, fourth or fifth aspect of the invention, the composition has an osmolarity of 268-290 mOsmoles/L, or an osmolarity of 275-289 mOsmoles/L. In one embodiment of the first, second, third, fourth or fifth aspect of the invention, the composition has an osmolarity of 268-298 mOsmoles/L. In one embodiment of the first, second, third, fourth or fifth aspect of the invention, the composition is isosmotic to human serum (about 290 mOsmoles/L). In one embodiment of the first, second, third, fourth or fifth aspect of the invention, the composition has a conductivity of 12.1-14.3 mS/cm. In one embodiment of the first, second, third, fourth or fifth aspect of the invention, the conductivity of the composition is comparable to that of human serum (about 12.6 mS/cm).

In one embodiment of the first, second, third, fourth or fifth aspect of the invention, the composition is a solution, suspension, dispersion, mixture or emulsion, or the composition comprises microdroplets. Typically the composition is an aqueous composition. In one embodiment, the composition is an aqueous solution, an aqueous suspension, an aqueous dispersion, an aqueous mixture or an aqueous emulsion, or is an aqueous composition comprising microdroplets. In one embodiment of the first, second, third, fourth or fifth aspect of the invention, the composition is provided in the form of a cream, gel, emulsion or ointment.

In one embodiment of the first, second, third, fourth or fifth aspect of the invention, the composition is sterile. In one embodiment of the first, second, third, fourth or fifth aspect of the invention, the composition is pharmaceutically acceptable.

In one embodiment of the first, second, third, fourth or fifth aspect of the invention, the composition is virucidal against a coronavirus (such as MERS-CoV, SARS-CoV, or SARS-CoV-2) or an influenza virus (such as influenza A virus (such as H1N1), influenza B virus, influenza C virus, or parainfluenza virus).

In one embodiment of the first, second, third, fourth or fifth aspect of the invention, the composition is suitable for inhibiting a protease (such as cathepsin L, 3CL, furin, or DPP4).

In one embodiment of the first, second, third, fourth or fifth aspect of the invention, the composition is suitable for inhibiting the interaction between the spike protein of a coronavirus (such as MERS-CoV, SARS-CoV, or SARS-CoV-2) and host receptors (such as ACE2 receptors).

In one embodiment of the second or fifth aspect of the invention, the aqueous composition is suitable for use in the treatment, prophylactic treatment or amelioration of an airborne viral infection, or for use in reducing or preventing (typically reducing) viral replication in a subject infected with an airborne virus or exposed to an airborne virus capable of causing an airborne viral infection in the subject.

In one embodiment of the first, second, third, fourth or fifth aspect of the invention, the composition is suitable for inhalation into the oral cavity, upper respiratory tract, lower respiratory tract, nasal cavity, pharynx, larynx, trachea, bronchi or lungs. In one embodiment of the first, second, third, fourth or fifth aspect of the invention, the composition is suitable for administration by inhaler, nebulizer, nasal spray, mouth spray, bronchial spray, nasal drops, nasal wash, nasal lavage, neti pot, nasal packing, mouthwash or gargle, or in the form of a cream, gel, emulsion, ointment, or any combination of these and similar methods of application. In one embodiment of the first, second, third, fourth or fifth aspect of the invention, the composition is suitable for application to a mask or other face covering. In one embodiment of the first, second, third, fourth or fifth aspect of the invention, the composition is suitable for use in a receptacle configured to allow an oxygen-containing gas to be bubbled through the composition prior to inhalation of the gas by a subject. In one embodiment, the composition is contained in a receptacle in which an oxygen-containing gas is bubbled through the composition prior to inhalation of the gas by a subject. In one embodiment, the receptacle can be part of a ventilator or a medical oxygen supply system, and typically the oxygen-containing gas comprises about 95% oxygen and about 5% carbon dioxide. In another embodiment, the receptacle is part of an air conditioner, and typically the oxygen-containing gas is air. In one embodiment of the first, second, third, fourth or fifth aspect of the invention, the composition is suitable for diffusing or spraying into the air prior to inhalation of the air by the subject. The air may be air in a building (e.g. a hotel, office, or retail, entertainment or sports venue) or a vehicle (e.g. a car, lorry, bus, tram, plane, train, boat or ship).

In one embodiment of the first, second, third, fourth or fifth aspect of the invention, the airborne viral infection is caused by an RNA virus, such as a coronavirus, an influenza virus, a rhinovirus, Measles virus, Mumps virus, Rubella virus, or human respiratory syncytial virus. In one embodiment, the airborne viral infection is caused by an RNA virus, such as a coronavirus, an influenza virus, a rhinovirus, Measles virus, or Mumps virus. In one embodiment, the airborne viral infection is caused by a coronavirus selected from MERS-CoV, SARS-CoV, and SARS-CoV-2. In a preferred embodiment, the airborne viral infection is caused by SARS-CoV-2. In one embodiment, the airborne viral infection is caused by an influenza virus selected from influenza A virus, influenza B virus, influenza C virus, and parainfluenza virus.

In another embodiment of the first, second, third, fourth or fifth aspect of the invention, the airborne viral infection is caused by a DNA virus, such as Parvovirus B19, an adenovirus, an adeno-associated virus, a herpes virus, a polyomavirus, or Variola virus. In one embodiment, the airborne viral infection is caused by a DNA virus, such as Epstein-Barr virus or Parvovirus B19. In one embodiment, the airborne viral infection is caused by a herpes virus selected from Varicella-Zoster virus (VZV or HHV-3), Epstein-Barr virus (EBV or HHV-4), human herpes virus 6 (HHV-6A and HHV-6B), and human herpes virus 7 (HHV-7). In one embodiment, the airborne viral infection is not caused by a herpes virus. In one embodiment, the airborne viral infection is caused by a polyomavirus selected from BK polyomavirus and WU polyomavirus.

A sixth aspect of the invention provides a method of preparing the composition of the first, second, third, fourth or fifth aspect of the invention, the method comprising combining all components with a solvent (preferably water), optionally making up to the desired volume, optionally filtering and optionally storing in a sealed vessel. In one embodiment, the method further comprises agitation of the composition, for example by stirring or shaking. In one embodiment, the sealed vessel is sterile. In one embodiment, the sealed vessel is impermeable to oxygen, carbon dioxide and water vapour.

A seventh aspect of the invention provides a concentrate for the preparation of an aqueous composition of the first, second, third, fourth or fifth aspect of the invention, the concentrate comprising all components and water, wherein the concentrate is dilutable with water to form the aqueous composition of the first, second, third, fourth or fifth aspect of the invention. In one embodiment, the concentrate is concentrated by a factor of from 1 to 50, or by a factor of from 5 to 20 compared to the aqueous composition of the first, second, third, fourth or fifth aspect of the invention. In one embodiment, the concentrate is sterile.

In one embodiment, the concentrate of the seventh aspect of the invention may be an aqueous concentrate comprising:

| (i) | from 10 to 1000 mmoles/L (preferably from 10 to 120 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | calcium ions and magnesium ions at a molar concentration ratio of from 5:1 to | |
| | 1:1, wherein said calcium ions are at a concentration of from 1.0 to 25 | |
| | mmoles/L; | |
| (iii) | from 210 to 350 mmoles/L | hydrogen carbonate ions or an equivalent |
| | | thereof; |
| (iv) | from 25 to 62 mmoles/L | potassium ions; |
| (v) | from 960 to 1260 mmoles/L | chloride ions; |
| (vi) | from 1000 to 1500 mmoles/L | sodium ions; and |
| (vii) | optionally from 1 to 2000 μmoles/L | zinc ions. |

As will be understood, in such an embodiment the concentrate may be diluted 10-fold with water to form an aqueous composition of the first, second, third, fourth or fifth aspect of the invention.

In another embodiment, the concentrate of the seventh aspect of the invention may be an aqueous concentrate comprising:

| (i) | from 10 to 120 mmoles/L | N-substituted aminosulphonic acid; |
|---|---|---|
| (ii) | calcium ions and magnesium ions at a molar concentration ratio of from 5:1 to | |
| | 1:1, wherein said calcium ions are at a concentration of from 1.0 to 25 | |
| | mmoles/L; | |
| (iii) | from 210 to 350 mmoles/L | hydrogen carbonate ions; |
| (iv) | from 25 to 62 mmoles/L | potassium ions; |
| (v) | from 960 to 1260 mmoles/L | chloride ions; |
| (vi) | from 1000 to 1500 mmoles/L | sodium ions; and |
| (vii) | optionally from 1 to 2000 μmoles/L | zinc ions. |

As will be understood, in such an embodiment the concentrate may be diluted 10-fold with water to form an aqueous composition of the first, second, third, fourth or fifth aspect of the invention.

An eighth aspect of the invention provides a concentrate for the preparation of an aqueous composition of the first, second, third, fourth or fifth aspect of the invention, the concentrate comprising water and all components except the hydrogen carbonate ions or the equivalent thereof and their countercations, wherein the concentrate is dilutable with water comprising the hydrogen carbonate ions or the equivalent thereof and their countercations to form the aqueous composition of the first, second, third, fourth or fifth aspect of the invention. In one embodiment, the concentrate is concentrated by a factor of from 1 to 50, or by a factor of from 5 to 20 compared to the aqueous composition of the first, second, third, fourth or fifth aspect of the invention. In one embodiment, the concentrate is sterile. The water comprising hydrogen carbonate ions and their countercations may be prepared by dissolving a hydrogen carbonate salt, such as sodium hydrogen carbonate, in water. The water comprising the equivalent of hydrogen carbonate ions and their countercations may be prepared by dissolving a salt of the equivalent, such as sodium acetate, in water.

In one embodiment, the concentrate of the eighth aspect of the invention may be an aqueous concentrate comprising:

| (i) | from 10 to 1000 mmoles/L (preferably from 10 to 120 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | calcium ions and magnesium ions at a molar concentration ratio of from 5:1 to | |
| | 1:1, wherein said calcium ions are at a concentration of from 1.0 to 25 | |
| | mmoles/L; | |
| (iii) | from 25 to 62 mmoles/L | potassium ions; |
| (iv) | from 960 to 1260 mmoles/L | chloride ions; |
| (v) | from 1000 to 1500 mmoles/L | sodium ions; and |
| (vi) | optionally from 1 to 2000 μmoles/L | zinc ions. |

As will be understood, in such an embodiment the concentrate may be diluted 10-fold with water comprising hydrogen carbonate ions or an equivalent thereof and their countercations, to form an aqueous composition of the first, second, third, fourth or fifth aspect of the invention.

In another embodiment, the concentrate of the eighth aspect of the invention may be an aqueous concentrate comprising:

| (i) | from 10 to 120 mmoles/L | N-substituted aminosulphonic acid; |
|---|---|---|
| (ii) | calcium ions and magnesium ions at a molar concentration ratio of from 5:1 to | |
| | 1:1, wherein said calcium ions are at a concentration of from 1.0 to 25 | |
| | mmoles/L; | |
| (iii) | from 25 to 62 mmoles/L | potassium ions; |
| (iv) | from 960 to 1260 mmoles/L | chloride ions; |
| (v) | from 1000 to 1500 mmoles/L | sodium ions; and |
| (vi) | optionally from 1 to 2000 μmoles/L | zinc ions. |

As will be understood, in such an embodiment the concentrate may be diluted 10-fold with water comprising hydrogen carbonate ions or an equivalent thereof and their countercations, to form an aqueous composition of the first, second, third, fourth or fifth aspect of the invention.

A ninth aspect of the invention provides a method of treatment, prophylactic treatment or amelioration of an airborne viral infection in a subject, the method comprising administering an effective amount of the composition of the first, second, third, fourth or fifth aspect of the invention to the subject.

The ninth aspect of the invention also provides a method of reducing or preventing (typically reducing) viral replication in a subject infected with an airborne virus or exposed to an airborne virus capable of causing an airborne viral infection in the subject, the method comprising administering an effective amount of the composition of the first, second, third, fourth or fifth aspect of the invention to the subject.

In one embodiment of the ninth aspect of the invention, the composition of the first, second, third, fourth or fifth aspect of the invention is applied to the oral cavity, the upper respiratory tract (including the nasal cavity, pharynx and/or larynx) and/or the lower respiratory tract (including the trachea, bronchi and/or lungs) of the subject, for example, by using a spray, inhaler, nebulizer, nasal spray, mouth spray, bronchial spray, nasal drops, nasal wash, nasal lavage, neti pot, nasal packing, mouthwash or gargle, cream, gel, emulsion, ointment, or any combination of these and similar methods of application. In one embodiment, the composition is applied once a day, twice a day, three times a day, or four times a day, or more. Without wishing to be bound by theory, it is currently believed that the hydrogen carbonate ions in the composition may be replenished during the use of the composition by the carbon dioxide in exhaled air. This may reduce the administration frequency, which would otherwise be required. Without wishing to be bound by theory, it is currently believed that the composition of the present invention may not just coat the subject's cell membranes, but may also enter the interstitial fluid which increases the effectiveness of the composition.

In one embodiment of the ninth aspect of the invention, the subject is a mammal, preferably a human.

In one embodiment of the ninth aspect of the invention, the airborne viral infection is caused by an RNA virus, such as a coronavirus, an influenza virus, a rhinovirus, Measles virus, Mumps virus, Rubella virus, or human respiratory syncytial virus. In one embodiment, the airborne viral infection is caused by an RNA virus, such as a coronavirus, an influenza virus, a rhinovirus, Measles virus, or Mumps virus. In one embodiment, the airborne viral infection is caused by a coronavirus selected from MERS-CoV, SARS-CoV, and SARS-CoV-2. In a preferred embodiment, the airborne viral infection is caused by SARS-CoV-2. In one embodiment, the airborne viral infection is caused by an influenza virus selected from influenza A virus, influenza B virus, influenza C virus, and parainfluenza virus.

In another embodiment of the ninth aspect of the invention, the airborne viral infection is caused by a DNA virus, such as Parvovirus B19, an adenovirus, an adeno-associated virus, a herpes virus, a polyomavirus, or Variola virus. In one embodiment, the airborne viral infection is caused by a DNA virus, such as Epstein-Barr virus or Parvovirus B19. In one embodiment, the airborne viral infection is caused by a herpes virus selected from Varicella-Zoster virus (VZV or HHV-3), Epstein-Barr virus (EBV or HHV-4), human herpes virus 6 (HHV-6A and HHV-6B), and human herpes virus 7 (HHV-7). In one embodiment, the airborne viral infection is not caused by a herpes virus. In one embodiment, the airborne viral infection is caused by a polyomavirus selected from BK polyomavirus and WU polyomavirus.

A tenth aspect of the invention provides a method of reducing the risk of viral infection with an airborne virus in a subject, the method comprising applying the composition of the first, second, third, fourth or fifth aspect of the invention to a mask or other face covering. Typically, the composition is applied to a part of the mask or face covering through which the subject breathes.

The tenth aspect of the invention also provides a method of reducing the risk of viral infection with an airborne virus in a subject, the method comprising bubbling an oxygen-containing gas through a composition of the first, second, third, fourth or fifth aspect of the invention prior to inhalation of the gas by the subject. Typically the composition is contained in a receptacle and the oxygen-containing gas is bubbled through the composition in the receptacle. In one embodiment, the receptacle can be part of a ventilator or a medical oxygen supply system. In one embodiment, the oxygen-containing gas comprises about 95% oxygen and about 5% carbon dioxide. In another embodiment, the receptacle is part of an air conditioner, and the oxygen-containing gas is air.

The tenth aspect of the invention also provides a method of reducing the risk of viral infection with an airborne virus in a subject, the method comprising diffusing or spraying the composition of the first, second, third, fourth or fifth aspect of the invention into the air prior to inhalation of the air by the subject. The air may be air in a building (e.g. a hotel, office, or retail, entertainment or sports venue) or a vehicle (e.g. a car, lorry, bus, tram, plane, train, boat or ship).

An eleventh aspect of the invention provides a spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising an aqueous composition comprising (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; and (ii) hydrogen carbonate ions or an equivalent thereof.

The eleventh aspect of the invention also provides a spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising an aqueous composition comprising an N-substituted aminosulphonic acid and hydrogen carbonate ions.

The eleventh aspect of the invention also provides a spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising the composition of the first, second, third, fourth or fifth aspect of the invention.

The eleventh aspect of the invention also provides a spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising (i) the concentrate of the seventh aspect of the invention, and (ii) water, wherein parts (i) and (ii) may be separate or together in the spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment.

The eleventh aspect of the invention also provides a spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising (i) the concentrate of the eighth aspect of the invention, and (ii) water comprising hydrogen carbonate ions or an equivalent thereof and their countercations, wherein parts (i) and (ii) may be separate or together in the spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment.

The spray of the eleventh aspect of the invention can be used to apply the composition of the first, second, third, fourth or fifth aspect of the invention to a mask or other face covering.

The spray, nasal spray, mouth spray, nasal drops, nasal wash, nasal lavage, neti pot, nasal packing, mouthwash or gargle, cream, gel, emulsion or ointment of the eleventh aspect of the invention can be used to administer the composition of the first, second, third, fourth or fifth aspect of the invention to the oral cavity and/or the upper respiratory tract (including the nasal cavity, pharynx and/or larynx) of a subject.

The inhaler, nebulizer or bronchial spray of the eleventh aspect of the invention can be used to administer the composition of the first, second, third, fourth or fifth aspect of the invention to the oral cavity, the upper respiratory tract (including the nasal cavity, pharynx and/or larynx) and/or the lower respiratory tract (including the trachea, bronchi and/or lungs) of a subject. In one embodiment, the nebulizer is a Vibrating Mesh Nebuliser (VMN), a Jet Nebuliser (JN) or an Ultrasonic Nebulizer. In one embodiment, the nebulizer is a Vibrating Mesh Nebuliser (VMN).

A twelfth aspect of the invention provides a mask or other face covering coated or impregnated with an aqueous composition comprising (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; and (ii) hydrogen carbonate ions or an equivalent thereof.

The twelfth aspect of the invention also provides a mask or other face covering coated or impregnated with an aqueous composition comprising an N-substituted aminosulphonic acid and hydrogen carbonate ions.

The twelfth aspect of the invention also provides a mask or other face covering coated or impregnated with the composition of the first, second, third, fourth or fifth aspect of the invention.

In one embodiment of the twelfth aspect of the invention, the mask or other face covering comprises an air permeable material, wherein the air permeable material is coated or impregnated with the composition, and wherein the mask or other face covering is configured to allow the wearer to breathe through the air permeable material.

A thirteenth aspect of the invention provides a method of cleaning an object, the method comprising applying the composition of the first, second, third, fourth or fifth aspect of the invention to the object. In a preferred embodiment the composition comprises an essential oil, or an extract or component thereof, with antiviral, antibacterial or antifungal activity. Preferably the essential oil, or the extract or component thereof, is selected from clove oil, eucalyptus oil, basil oil, ginger oil, saffron oil, cannabis oil, tea tree oil, black seed oil, or an extract or component of any of these. Preferably the essential oil, or the extract or component thereof, is selected from clove oil, eucalyptus oil, basil oil, ginger oil, or an extract or component of any of these. In a preferred embodiment the composition further comprises a buffer.

The object to be cleaned can be anything from which it is desired to remove or reduce the amount of viruses, bacteria and/or fungi. Typical objects are pens, counters, shopping carts, tables, doorknobs, light switches, handles, stair rails, hand rails, elevator buttons, desks, keyboards, phones, toilets, faucets, sinks, and other objects that are or have high-frequency touch points.

In one embodiment of the first to thirteenth aspect of the invention, the water used is distilled water. In another embodiment of the first to thirteenth aspect of the invention, the water used is sterile water. In another embodiment of the first to thirteenth aspect of the invention, the water used is ultrapure water.

### Definitions

For the purposes of the present invention, the term “anti-infective” includes antimicrobial, antiviral and virucidal, wherein the term “antimicrobial” includes antibacterial and antifungal. The term “infection” refers to a disease, disorder or condition caused by an “infectious agent”, wherein the infectious agent can be a bacterium, fungus or virus.

A “virucidal” agent inactivates viruses so that they cannot enter host cells, either by killing them or by changing their surface structure. If a virus loses the ability to replicate because of an agent, then the agent is “antiviral”.

For the purposes of the present invention, an “airborne viral infection” is an infection transmitted by an airborne virus. “Airborne viruses” are those in which the disease spreads in particles in exhaled air. These particles include aerosols, which are smaller than 5 micrometres in diameter and which can remain airborne for extended periods, and also larger droplets through which transmission can occur over relatively short distances. Airborne viruses include (i) RNA viruses, such as coronaviruses (such as MERS-CoV, SARS-CoV, and SARS-CoV-2), influenza viruses (such as influenza A virus, influenza B virus, influenza C virus, and parainfluenza virus), rhinoviruses, Measles virus, Mumps virus, Rubella virus, and human respiratory syncytial virus, and (ii) DNA viruses, such as Parvovirus B19, adenoviruses and adeno-associated viruses, herpes viruses (such as Varicella-Zoster virus (VZV or HHV-3), Epstein-Barr virus (EBV or HHV-4), human herpes virus 6 (HHV-6A and HHV-6B), and human herpes virus 7 (HHV-7)), polyomaviruses (such as BK polyomavirus and WU polyomavirus), and Variola virus.

Airborne viral infections cause a variety of diseases, for example, SARS-CoV-2 causes COVID-19, influenza viruses cause the flu, and various airborne viruses can cause viral tonsillitis.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of a viral infection, the stabilisation (i.e., not worsening) of a viral infection, the delay or slowing of progression/worsening of a viral infection/symptom, the amelioration or palliation of a viral infection/symptom, and remission (whether partial or total), whether detectable or undetectable. The term “amelioration” as used herein means that the extent and/or undesirable manifestations of a viral infection or symptom are lessened and/or the time course of the progression is slowed or lengthened, as compared to not administering a composition of the invention. The term “prophylactic treatment” as used herein relates to preventative therapy, as well as therapy to reduce the risk of developing a viral infection. The term “prophylactic treatment” includes both the avoidance of occurrence of a viral infection, and the delay in onset of a viral infection.

An “alkyl” group may be linear (i.e. straight-chained) or branched. Examples of alkyl groups include methyl, ethyl, n-propyl, i-propyl, n-butyl, i-butyl, t-butyl and n-pentyl groups. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

Unless stated otherwise, where a group is prefixed by the term “halo”, such as a “haloalkyl” or “halomethyl” group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Similarly, where a group is prefixed by the term “hydroxy”, such as a “hydroxyalkyl” group, it is to be understood that the group in question is substituted with one or more (such as one, two, three, four or five) hydroxy groups.

An “alkenyl” group refers to an unsaturated alkyl group having one or more carbon-carbon double bonds. Examples of alkenyl groups include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” group refers to an unsaturated alkyl group having one or more carbon-carbon triple bonds. Examples of alkynyl groups include ethynyl, propargyl, but-1-ynyl and but-2-ynyl groups. Typically an alkynyl group is a C₂-C₁₂ alkynyl group. More typically an alkynyl group is a C₂-C₆ alkynyl group. An “alkynylene” group is similarly defined as a divalent alkynyl group.

A “cycloalkyl” group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl group may include monocyclic, bicyclic or polycyclic hydrocarbyl rings.

A “cycloalkenyl” group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl group may include monocyclic, bicyclic or polycyclic hydrocarbyl rings.

A “heterocyclic group” is a non-aromatic cyclic group including one or more carbon atoms and one or more (such as one, two, three or four) heteroatoms, e.g. N, O or S, in the ring structure. Examples of heterocyclic groups are azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups. In one embodiment, a heterocyclic group is 4- to 7-membered. In another embodiment, a heterocyclic group is 5- to 6-membered.

A “heteroaryl group” is an aromatic cyclic group including one or more carbon atoms and one or more (such as one, two, three or four) heteroatoms, e.g. N, O or S, in the ring structure. Examples of heteroaryl groups are pyridinyl, pyridazinyl, pyrimidinyl, pyrazinyl, triazinyl, pyrrolyl, furanyl, thiophenyl, pyrazolyl, imidazolyl, oxazolyl, isoxazolyl, thiazolyl, isothiazolyl, triazolyl, oxadiazolyl and thiadiazolyl groups. In one embodiment, a heteroaryl group is 5- to 6-membered.

The term “halo” includes fluoro, chloro, bromo and iodo.

For the purposes of the present specification, where it is stated that a first atom or group is “directly attached” to a second atom or group it is to be understood that the first atom or group is covalently bonded to the second atom or group with no intervening atom(s) or group(s) being present. For example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

A peptide comprises two or more amino acids linked by a peptide bond. A peptide is a polymer comprising two or more amino acid monomers. A peptide bond is an amide bond (—CO—NH—) formed between the carboxylic acid group of one amino acid and the amino group of another amino acid. For the purposes of the present invention, a “peptide equivalent” may comprise one or more amino acids, but it also comprises at least one amino acid equivalent, such as for example an amino sulphonic acid or an amino sulphinic acid. A peptide equivalent is a polymer which may comprise one or more amino acid monomers, but which also comprises at least one amino acid equivalent monomer, such as for example an amino sulphonic acid monomer or an amino sulphinic acid monomer. Therefore a peptide equivalent has either (i) a C-terminal amino acid equivalent or (ii) at least one bond linking two monomers which is not a peptide bond, for example, an amino sulphonic acid is linked to another monomer by a sulphonamide bond (—SO—NH—), or an amino sulphinic acid is linked to another monomer by a sulphinamide bond (—SO—NH—).

For the purposes of the present invention, pH can be measured as set out in the European Pharmacopoeia 5.0, volume 1, 2005, paragraph 2.2.3.

Buffer capacity (P) quantifies the ability of a buffer composition to resist changes in pH by either absorbing or desorbing H⁺ and OH⁻ ions. When an acid or base is added to a buffer composition, the effect on pH change can be large or small, depending on both the initial pH and the capacity of the buffer composition to resist change in pH. Buffer capacity (P) is defined as the moles of an acid or base necessary to change the pH of a buffer composition by 1, divided by the volume of the buffer composition in litres; it is a unitless number.

While some of the components of the compositions of the present invention are explicitly said to be present in the composition as ions (i.e. in ionic form), it will be understood that many other components may also be present in ionic form in the compositions of the present invention.

Any of the components of the compositions of the present invention may be present in salt form.

Any of the components of the compositions of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I. For example, histidine can be in the form of L-histidine-d₃ (α-d₁, imidazole-2,5-d₂).

In the context of the present invention, “substantially all” means 98% or more, or 99% or more, or 99.5% or more, or 99.9% or more, by weight.

In the context of the present invention, where a composition is said to be “substantially free” of a component, this means that the composition comprises 2% or less, or 1% or less, or 0.5% or less, or 0.1% or less of that component, by weight.

For the purposes of the present invention, an “enantiomerically enriched” isomer of a compound comprises less than 40% of other isomers of the same compound by weight.

An “enantiomerically pure” isomer of a compound comprises less than 5% of other isomers of the same compound, more typically less than 2%, and most typically less than 0.5% by weight.

### FIGURES OF THE INVENTION

FIG. 1 lists buffers of the invention. All of the buffers listed in FIG. 1 are considered to be Good's buffers. Good's buffers may be classified as N-substituted aminosulphonic acid Good's buffers and non-sulphonic acid Good's buffers, as indicated in FIG. 1.

FIG. 2 shows a SARS-CoV-2 growth curve analysis in infected Vero cells treated with test buffer or control medium. 1×10⁵ Vero cells per well were pre-treated with test buffer or control medium 1 hour prior to infection with SARS-CoV-2 at moi 0.1. Adsorption was carried out at 4° C. The infected monolayer was then washed twice with cold DMEM+0.2% BSA, followed by 1-hour incubation at 37° C. either in the presence of test buffer or control medium. After 1 hour, the monolayer was washed again with DMEM-2 (FBS 2%) to remove unbound virus. Cells were then incubated for the indicated times when virus yield was collected and titrated for infectivity. Graph and statistical analysis were performed with Graphpad Prism 6 software. Data was plotted as mean f SD from 3 replicates; *p<0.05; ***p<0.001; ****P<0.0001.

FIG. 3 shows the % neutralisation of SARS-CoV-2 spike pseudotype reporter particles by test buffers at various dilutions compared to control. SARS-CoV-2 spike pseudotype reporter particles were incubated with test buffers at various dilutions or control at 37° C. for 1 hour. Then 4×10⁴ HuH7 cells (human hepatocytes) were added and incubated at 37° C. for 72 hours, after which infection rates were determined by measuring luminescence/luciferase activity.

FIG. 4 shows the amount of eugenol permeated through goat nasal mucosa vs time using four compositions (composition 7B comprising eugenol, buffer and zinc ions; composition 7D comprising eugenol and buffer but without zinc ions; composition 7N comprising eugenol and zinc ions but without buffer; and composition 70 comprising eugenol but without buffer and without zinc ions). The slope gives the value of steady state flux (J).

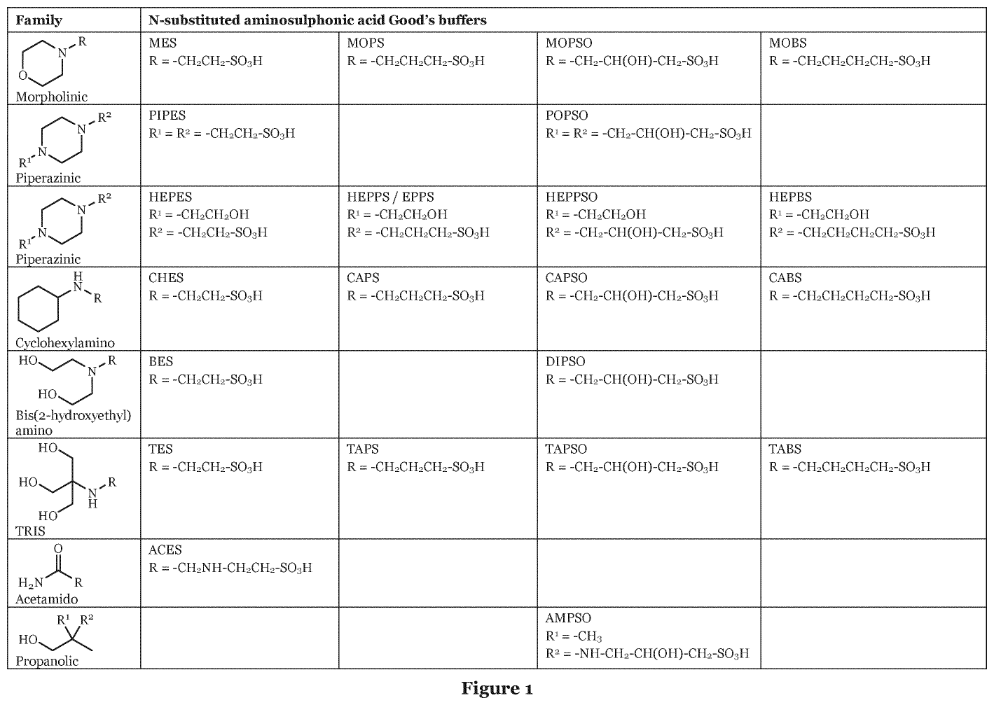

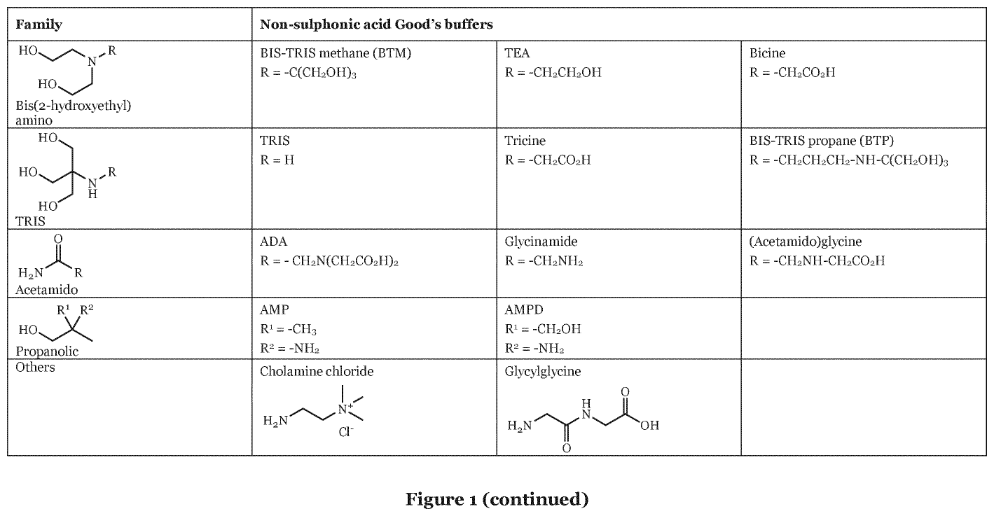

### EXAMPLES

### Example 1

An aqueous buffer solution was prepared by stirring the components set out in Table 1 in 1 L of sterile water.

TABLE 1

| | | |
|---|---|---|
| Weight | Molar | |
| Amount | Amount | Component |

| | | | | |
|---|---|---|---|---|
| 1.066 | g | 5 | mmoles | N,N-bis(2-hydroxyethyl)-2- |
| | | | | aminoethanesulphonic acid |
| | | | | (BES) |
| 138.725 | mg | 1.25 | mmoles | calcium chloride |
| 42.845 | mg | 0.45 | mmoles | magnesium chloride |
| 2.100 | g | 25 | mmoles | sodium hydrogen carbonate |
| 372.757 | mg | 5 | mmoles | potassium chloride |
| 6.428 | g | 110 | mmoles | sodium chloride |

### Example 2 (Hypothetic)

An aqueous buffer solution was prepared by stirring the components set out in Table 2 in 1 L of sterile water.

TABLE 2

| | | |
|---|---|---|
| Weight | Molar | |
| Amount | Amount | Component |

| | | | | |
|---|---|---|---|---|
| 1.066 | g | 5 | mmoles | N,N-bis(2-hydroxyethyl)-2- |
| | | | | aminoethanesulphonic acid |
| | | | | (BES) |
| 138.725 | mg | 1.25 | mmoles | calcium chloride |
| 42.845 | mg | 0.45 | mmoles | magnesium chloride |
| 2.100 | g | 25 | mmoles | sodium hydrogen carbonate |
| 372.757 | mg | 5 | mmoles | potassium chloride |
| 6.428 | g | 110 | mmoles | sodium chloride |
| 6.814 | mg | 50 | μmoles | zinc chloride |

### Example 3 (Hypothetic)

The capacity of the subject buffer solutions to prevent or reduce viral infection of human bronchial epithelial cells may be tested using a method such as that described by Harcourt et al (J Vis Exp, 2013, vol 72, e50157). In the disclosed method, a polarized layer of human airway epithelial Calu-3 cells may be prepared in liquid-covered cultures (LCC) in Transwells.

Polarisation of the LCC may be evaluated by determining the trans-epithelial electrical resistance (TEER) and/or equilibration of sodium fluorescein (both methods as described in Harcourt et al). Viral-induced depolarisation of the cell layer may be evaluated as described in Harcourt et al.

LCC are washed in serum-free medium, such as serum-free EMEM. The subject buffer solution is added to the basolateral compartments of all test Transwells. For “uninfected” control groups, the subject buffer solution is also added to the apical compartment of the appropriate Transwells. For “mock-infected” control groups, inactivated virus diluted in the subject buffer solution is added to the apical compartment of the appropriate Transwells, and for “virus-infected” test groups, virus diluted in the subject buffer solution is added to the apical compartments of appropriate Transwells.

The effect of the subject buffer solutions on virus-induced depolarisation of the cell layer may then be determined by measuring the TEER and/or equilibration of sodium fluorescein as described in Harcourt et al.

### Example 4

Aqueous buffer solutions A, B, C and D were prepared by stirring the components set out in Table 3 in 11 L of ultrapure water (ATSM Type 1, 18.2 MΩ/cm at 25° C.).

TABLE 3

| | | Buffer A | | Buffer B | | Buffer C | | Buffer D | |
|---|---|---|---|---|---|---|---|---|---|
| | Molecular | Molar | | Molar | | Molar | | Molar | |
| Component | weight | Amount | | Amount | | Amount | | Amount | |
| N,N-bis(2-hydroxyethyl)-2-amino- | 213.25 | 5.00 | mmoles | 5.00 | mmoles | 5.00 | mmoles | 5.00 | mmoles |
| ethanesulphonic acid (BES) | | | | | | | | | |
| CaCl₂•2 H₂O | 147.0146 | 1.25 | mmoles | 1.25 | mmoles | 1.25 | mmoles | 1.25 | mmoles |
| MgCl₂•6 H₂O | 203.30 | 0.45 | mmoles | 0.45 | mmoles | 0.45 | mmoles | 0.45 | mmoles |
| NaHCO₃ | 84.01 | 25.00 | mmoles | 25.00 | mmoles | 25.00 | mmoles | 25.00 | mmoles |
| KCl | 74.55 | 5.00 | mmoles | 5.00 | mmoles | 5.00 | mmoles | 5.00 | mmoles |
| NaCl | 58.50 | 110.00 | mmoles | 110.00 | mmoles | 110.00 | mmoles | 110.00 | mmoles |
| ZnCl₂ | 136.30 | — | | 0.1 | mmoles | — | | 0.1 | mmoles |
| D-glucose•H₂O | 198.17 | 10.00 | mmoles | 10.00 | mmoles | 10.00 | mmoles | 10.00 | mmoles |
| Glycerol | 92.0938 | 0.11 | mmoles | 0.11 | mmoles | 0.11 | mmoles | 0.11 | mmoles |
| L-Glutamic acid | 147.1293 | 0.30 | mmoles | 0.30 | mmoles | 0.30 | mmoles | 0.30 | mmoles |
| L-Glutamine | 146.146 | 0.40 | mmoles | 0.40 | mmoles | 0.40 | mmoles | 0.40 | mmoles |
| L-Aspartic acid | 133.11 | 0.02 | mmoles | 0.02 | mmoles | 0.02 | mmoles | 0.02 | mmoles |
| L-Carnitine ‘inner salt’ | 161.20 | 0.05 | mmoles | 0.05 | mmoles | 0.05 | mmoles | 0.05 | mmoles |
| Choline Chloride | 139.62 | 0.01 | mmoles | 0.01 | mmoles | 0.01 | mmoles | 0.01 | mmoles |

| | | | | | | | |
|---|---|---|---|---|---|---|---|
| Thiamine Pyrophosphate Chloride | 460.77 | — | — | 40.00 | nmoles | 40.00 | nmoles |
| Human recombinant insulin | | — | — | 28 | mIU | 28 | mIU |

| | | | | |
|---|---|---|---|---|
| Measured Osmolarity [mOsmoles/L] | 270 | 272 | 286 | 288 |
| Conductivity [mS/cm] | 12.33 | 12.37 | 12.21 | 12.25 |
| pH at 37° C. | 7.29 | 7.15 | 7.30 | 7.22 |
| Viscosity at 20° C. [centipoise] | | 0.91 | | |

### Example 5—Antiviral Trial Test with Buffers to Assess Coronavirus Entry Interference

Host: Vero-CCL81

Virus: SARS-CoV-2 (stock titer 1.67×10⁶ PFU/mL in Vero) (PFU=plaque forming unit)

I. Objective: Infection of Vero cells with SARS-CoV-2, either in the presence or absence of test buffers designed to prevent (or decrease) endosome acidification and virus entry into cells.

I. Study Design:
1. 7e4 cells were seeded into each well of a 24-well plate the day before testing. 24 hours later, right before infection, the number of cells counted per well was 1e5.
2. Moi (multiplicity of infection) used in the experiment was 0.1 (1 PFU to every 10 cells).
3. 1 hour prior to infection, cells were pre-treated with test buffer or control medium by replacing overlay media (DMEM-10) with 0.5 mL of test buffer or control medium.
4. The adsorption was carried out at 4° C., with virus inoculum prepared in test buffer or control medium. Virus inoculum was prepared to have the calculated virus titer in 100 μL/well.
5. After adsorption, unbound virus was removed by washing the monolayer twice with cold binding buffer (DMEM+0.2% BSA).
6. Following adsorption, infected cells were incubated for 1 hour at 37° C. to synchronize infection and to promote entry, either in the presence of test buffer or control medium.
7. After 1 hour, the monolayer was washed 3 times with DMEM-2 (FBS 2%), an extra step to remove unbound virus. Then the monolayer was covered with 0.5 mL of DMEM-2 for the times indicated below when virus in the overlay was collected and titrated for infectivity through plaque assay in Vero cells.
8. Time points: 1 (after 1 hour incubation at 37° C.), 3, 6, 24 and 48 hours post infection (hpi).
9. Graph and statistical analysis were performed with Graphpad Prism 6 software. Data was plotted as mean f SD from 3 replicates; *p<0.05; ***p<0.001; ****P<0.0001.

III. Conditions:
Trial A. medium=serum free low pH=5.5 adjusted control (negative control)
   Forced entry
Trial B. medium=serum free low pH+100 mM NH₄Cl (positive control)
   Blocked entry
   Note: Vero cells got rounded and detached from the well after treatment with low pH media. Since the protocol involves many washing steps, too many cells were lost. The remaining cells recovered after replacement of low pH media with DMEM-2, but their total numbers were too different for a statistical comparison with the other groups. The same was not observed with the test buffers, Vero cells showed no cytotoxic signs after treatment with the test buffers.
Trial C. medium=buffer A of example 4
Trial D. medium=buffer B of example 4
Trial E. medium=buffer C of example 4
Trial F. medium=buffer D of example 4
Trial G. medium=Uninfected control—DMEM-2
   Monolayer of Vero cells was intact and showed no signs of damage or cytotoxicity after treatment with DMEM-2
Trial H.=Untreated control. Infected moi 0.1—DMEM-2
   24-well Plate Design (one plate per time point):

| | 1 | 2 | 3 | 4 | 5 | 6 |
|---|---|---|---|---|---|---|
| A | A | A | A | E | E | E |
| B | B | B | B | F | F | F |
| C | C | C | C | G | G | G |
| D | D | D | D | H | H | H |

IV. Virus: Sars-CoV-2 titrated in Vero cells on the day of experiment.
Plaque counts:19 PFUs at dilution 10-4 (10-fold dilution)
Titer on day of infection: 1.9×10⁶ PFU/ml

V. Results:

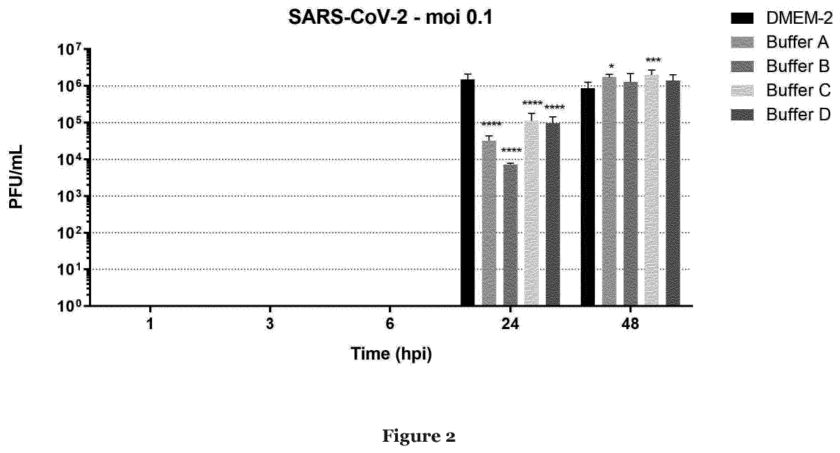

The results are represented graphically in FIG. 2 which shows a SARS-CoV-2 growth curve analysis in infected Vero cells treated with test buffer or control medium. As can be seen, all four test buffers of example 4 suppressed viral replication for at least 24 hours to a statistically significant extent compared to untreated control in DMEM-2.

### Example 6—Insight into Mode of Action

Host: HuH7 cells (human hepatocytes)

Virus: SARS-CoV-2 spike pseudotype reporter particle

I. Objectives: Test for effect of buffers to prevent or decrease viral entry/first cycle of replication. Investigate the effect of reducing ZnCl₂ concentration in buffers (100 μM to 0.39 μM)

II. Study Design:

Entry of coronavirus into host cells is mediated by the spike S protein. SARS-CoV-2 spike pseudotype reporter particles were used as a model that simulates infection by SARS-CoV-2 by replacing the envelope proteins in the vector virus with the spike S protein. The vector virus contains a reporter luminescent gene. By detecting the luminescence in target cells, it is possible to screen the ability of a test buffer to neutralize the virus i.e. the ability of a test buffer to reduce viral infectivity. Therefore this experiment investigates the early stage of infection, i.e. entry and first cycle of replication only.

Dilution of buffers and incubation with pseudovirus:
1. In a flat-bottom 96-well plate (ThermoFisher, #136101), dilutions of buffers A and B of example 4 (in duplicate) were prepared to a final dilution of 1/256 of buffer in a total volume of 100 μl per well.
2. 1×10⁵ RLU of SARS-CoV-2 pseudotyped lentiviral particles were added to each well and incubated at 37° C. for 1 hour.
3. The first column (8 control wells) received SARS-CoV-2 pseudotyped lentiviral particles and cells only (virus control) and the second column received cells only (background control).

Preparation of HuH7 cells and challenge:
1. Media was removed from T75 containing adherent HuH7 cells and the cells were rinsed with PBS.
2. 2 ml of trypsin was added to the cells and the cells were returned to the incubator for 5 minutes.
3. It was confirmed using a microscope that cells had become detached and the cells were resuspended in 8 ml of FBS-containing DMEM, pipetting up and down to produce a suspension of single cells.
4. The concentration of cells was determined using a cell counter.
5. The cells were diluted in DMEM to a final concentration of 4×10⁵ cells/ml.
6. 100 μl of cell suspension containing 4×10⁴ cells was added to each well of the 96-well plate.
7. The plate was incubated at 37° C. and 5% CO₂ for 72 hours.

Assessing neutralisation of infection by buffers:
1. 150 μl of media/supernatant was removed from each well and 50 μl of Steady-Glo® Luciferase Assay System (Promega) was added.
2. Luminescence/luciferase activity was measured using CLARIOstar Plate Reader (BMG Labtech).

Analysis using PRISM 8:
1. The curves of relative infection rates (in %) versus the buffer dilutions (log 10 values) against a negative control of pooled sera collected prior to 2016 (Sigma) and serum of a positive neutraliser were plotted using Prism 8 (GraphPad).
2. A non-linear regression (curve fit) method was used to determine the dilution fold that neutralised 50%.

II. Results:

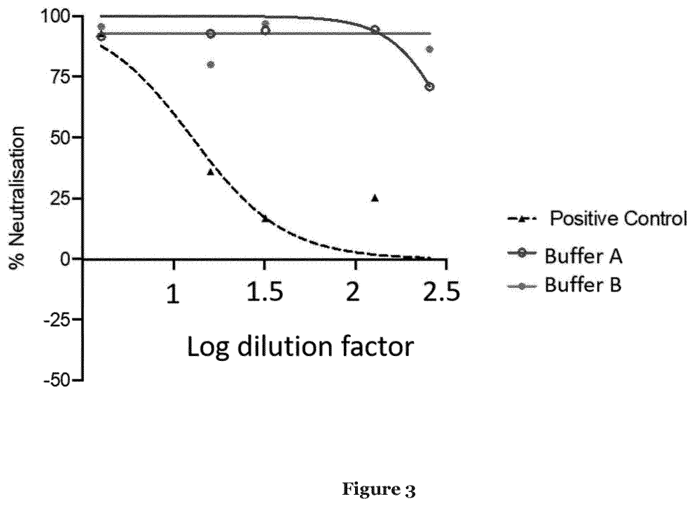

The results are represented graphically in FIG. 3 which shows the % neutralisation versus the buffer dilutions (log 10 values). As can be seen, buffers A and B of example 4 effectively inhibited the infection of HuH7 cells with SARS-CoV-2 spike pseudotype reporter particles, even at 256× dilution.

### Example 7—Synthetic

Eight compositions according to the invention were prepared as follows. Compositions A, B and C vary in the amount of essential oils (ginger oil, eucalyptus oil, basil oil, and clove oil) they comprise, namely a total of 2%, 1% and 0.4% respectively. Composition D is the same as composition B, but without zinc chloride. Compositions E, F, G and H vary in the type of essential oil, each composition comprising only one of ginger oil, eucalyptus oil, basil oil or clove oil respectively.

TABLE 4
components of phosphate buffer

| Component | Quantity | |
|---|---|---|
| KH₂PO₄ | 0.3532 | g |
| Na₂HPO₄ | 1.4542 | g |
| Water for injection | Qs | |
| Total | 100 | ml |

TABLE 5A

| | Quantity | | Quantity | | Quantity | | Quantity | |
|---|---|---|---|---|---|---|---|---|
| Component | Composition A | | Composition B | | Composition C | | Composition D | |
| NaCl | 0.6 | g | 0.6 | g | 0.6 | g | 0.6 | g |
| KCl | 0.0075 | g | 0.0075 | g | 0.0075 | g | 0.0075 | g |
| MgCl₂•6 H₂O | 0.009 | g | 0.009 | g | 0.009 | g | 0.009 | g |
| NaHCO₃ | 0.21 | g | 0.21 | g | 0.21 | g | 0.21 | g |
| Xylitol | 1 | g | 1 | g | 1 | g | 1 | g |
| EDTA | 0.1 | g | 0.1 | g | 0.1 | g | 0.1 | g |
| CaCl₂•2 H₂O | 0.0183 | g | 0.0183 | g | 0.0183 | g | 0.0183 | g |
| ZnCl₂ | 0.0001 | g | 0.0001 | g | 0.0001 | g | — | |
| Glycerol | 0.001 | g | 0.001 | g | 0.001 | g | 0.001 | g |
| HPMC | 0.5 | g | 0.5 | g | 0.5 | g | 0.5 | g |
| Ginger oil | 0.5 | g | 0.25 | g | 0.1 | g | 0.25 | g |
| Eucalyptus oil | 0.5 | g | 0.25 | g | 0.1 | g | 0.25 | g |
| Basil oil | 0.5 | g | 0.25 | g | 0.1 | g | 0.25 | g |
| Clove oil | 0.5 | g | 0.25 | g | 0.1 | g | 0.25 | g |
| Water for injection | 40 | ml | 40 | ml | 40 | ml | 40 | ml |
| PEG 400 | 5 | ml | 5 | ml | 5 | ml | 5 | ml |
| Poloxamer 188 | 1.2 | g | 1.2 | g | 1.2 | g | 1.2 | g |
| Benzalkonium chloride | 0.01 | g | 0.01 | g | 0.01 | g | 0.01 | g |
| Phosphate Buffer | Qs | | Qs | | Qs | | Qs | |
| Sodium Hyaluronate | 0.2 | g | 0.2 | g | 0.2 | g | 0.2 | g |
| Total | 100 | ml | 100 | ml | 100 | ml | 100 | ml |

TABLE 5B

| | Quantity | | Quantity | | Quantity | | Quantity | |
|---|---|---|---|---|---|---|---|---|
| Component | Composition E | | Composition F | | Composition G | | Composition H | |
| NaCl | 0.6 | g | 0.6 | g | 0.6 | g | 0.6 | g |
| KCl | 0.0075 | g | 0.0075 | g | 0.0075 | g | 0.0075 | g |
| MgCl₂•6 H₂O | 0.009 | g | 0.009 | g | 0.009 | g | 0.009 | g |
| NaHCO₃ | 0.21 | g | 0.21 | g | 0.21 | g | 0.21 | g |
| Xylitol | 1 | g | 1 | g | 1 | g | 1 | g |
| EDTA | 0.1 | g | 0.1 | g | 0.1 | g | 0.1 | g |
| CaCl₂•2 H₂O | 0.0183 | g | 0.0183 | g | 0.0183 | g | 0.0183 | g |
| ZnCl₂ | 0.0001 | g | 0.0001 | g | 0.0001 | g | 0.0001 | g |
| Glycerol | 0.001 | g | 0.001 | g | 0.001 | g | 0.001 | g |
| HPMC | 0.5 | g | 0.5 | g | 0.5 | g | 0.5 | g |
| Ginger oil | 0.25 | g | — | | — | | — | |
| Eucalyptus oil | — | | 0.25 | g | — | | — | |
| Basil oil | — | | — | | 0.25 | g | — | |
| Clove oil | — | | — | | — | | 0.25 | g |
| Water for injection | 40 | ml | 40 | ml | 40 | ml | 40 | ml |
| PEG 400 | 5 | ml | 5 | ml | 5 | ml | 5 | ml |
| Poloxamer 188 | 1.2 | g | 1.2 | g | 1.2 | g | 1.2 | g |
| Benzalkonium chloride | 0.01 | g | 0.01 | g | 0.01 | g | 0.01 | g |
| Phosphate Buffer | Qs | | Qs | | Qs | | Qs | |
| Sodium Hyaluronate | 0.2 | g | 0.2 | g | 0.2 | g | 0.2 | g |
| Total | 100 | ml | 100 | ml | 100 | ml | 100 | ml |

Method of Manufacturing:

Phase I: Preparation of Phosphate Buffer
1. KH₂PO₄ was dissolved in water for injection (24 ml) to obtain a clear solution.
2. Na₂HPO₄ was added to the solution and stirred to obtain a clear solution.
3. The volume was made up to 100 ml with water for injection.
4. The pH of the buffer was checked and found to be in the range of 7.2 to 7.5.

Phase II: Preparation of Aqueous Phase
1. Poloxamer was dissolved in water for injection (10 ml). Then sodium hyaluronate was added and the mixture was allowed to swell to give mixture A.
2. HPMC was allowed to swell in water for injection (10 ml) to give mixture B.
3. NaCl, KCl, MgCl₂. 6 H₂O, NaHCO₃, xylitol, EDTA, CaCl₂·2 H₂O, and ZnCl₂ (where used), one after the other in this order, were dissolved in water for injection (20 ml) to give mixture C.

Phase III: Preparation of Oil Phase and Mixing
1. PEG 400 and all essential oils (ginger oil, eucalyptus oil, basil oil, and/or clove oil) were added together to give mixture D.
2. Mixtures A and B were mixed together, and then mixture C was added to form a blend.
3. Mixture D was added to this blend, and then glycerol and benzalkonium chloride were added.
4. The volume was made up to 100 ml with the previously prepared phosphate buffer.
5. This mixture was homogenized at 8000-9000 rpm for 15-20 minutes.
6. The homogenized composition was filtered through a Whatman filter (paper of size 11 μm).
7. The pH of the composition was checked and found to be in the range of 7.2 to 7.7. The buffer capacity with respect to 1M HCl was found to be 0.02. The viscosity of composition 7B was found to be 36 centipoise at 20° C.
8. The composition was dispensed into spray bottles.

Five further compositions (I-M) without buffer were prepared.

TABLE 5C

| | Quantity | | Quantity | | Quantity | | Quantity | | Quantity | |
|---|---|---|---|---|---|---|---|---|---|---|
| Component | Comp. I | | Comp. J | | Comp. K | | Comp. L | | Comp. M | |
| HPMC | 0.5 | g | 0.5 | g | 0.5 | g | 0.5 | g | 0.5 | g |
| Ginger oil | 0.25 | g | 0.25 | g | — | | — | | — | |
| Eucalyptus oil | 0.25 | g | — | | 0.25 | g | — | | — | |
| Basil oil | 0.25 | g | — | | — | | 0.25 | g | — | |
| Clove oil | 0.25 | g | — | | — | | — | | 0.25 | g |
| Water for injection | Qs | | Qs | | Qs | | Qs | | Qs | |
| Poloxamer 188 | 1.2 | g | 1.2 | g | 1.2 | g | 1.2 | g | 1.2 | g |
| Total | 100 | ml | 100 | ml | 100 | ml | 100 | ml | 100 | ml |

Method of Manufacturing:
1. Poloxamer was dissolved in water for injection (10 ml) to give mixture A.
2. HPMC was allowed to swell in water for injection (10 ml) to give mixture B.
3. Mixtures A and B were mixed together.
4. The essential oil (ginger oil, eucalyptus oil, basil oil, and/or clove oil) was added to this blend.
5. The volume was made up to 100 ml with water for injection.
6. This mixture was homogenized at 8000-9000 rpm for 15-20 minutes.
7. The homogenized composition was filtered through a Whatman filter (paper of size 11 μm).
8. The pH of the composition was checked and found to be in the range of 6.7-7.0.
9. The composition was dispensed into spray bottles.

Two further compositions (N and O) without buffer were prepared.

TABLE 5D

| | | |
|---|---|---|
| | Quantity | Quantity |
| Component | Composition N | Composition O |

| NaCl | 0.6 | g | 0.6 | g |
|---|---|---|---|---|
| KCl | 0.0075 | g | 0.0075 | g |
| MgCl₂•6 H₂O | 0.009 | g | 0.009 | g |
| Xylitol | 1 | g | 1 | g |
| EDTA | 0.1 | g | 0.1 | g |
| CaCl₂•2 H₂O | 0.0183 | g | 0.0183 | g |
| ZnCl₂ | 0.0001 | g | — | |
| Glycerol | 0.001 | g | 0.001 | g |
| HPMC | 0.5 | g | 0.5 | g |
| Ginger oil | 0.25 | g | 0.25 | g |
| Eucalyptus oil | 0.25 | g | 0.25 | g |
| Basil oil | 0.25 | g | 0.25 | g |
| Clove oil | 0.25 | g | 0.25 | g |

| | | |
|---|---|---|
| Water for injection | Qs | Qs |

| | | | | |
|---|---|---|---|---|
| PEG 400 | 5 | ml | 5 | ml |
| Poloxamer 188 | 1.2 | g | 1.2 | g |
| Benzalkonium chloride | 0.01 | g | 0.01 | g |
| Sodium Hyaluronate | 0.2 | g | 0.2 | g |
| Total | 100 | ml | 100 | ml |

Method of Manufacturing:

Phase I: Preparation of Aqueous Phase
1. Poloxamer was dissolved in water for injection (10 ml). Then sodium hyaluronate was added and the mixture was allowed to swell to give mixture A.
2. HPMC was allowed to swell in water for injection (10 ml) to give mixture B.
3. NaCl, KCl, MgCl₂. 6 H₂O, xylitol, EDTA, CaCl₂·2 H₂O, and ZnCl₂ (where used), one after the other in this order, were dissolved in water for injection (20 ml) to give mixture C.

Phase II: Preparation of Oil Phase and Mixing
1. PEG 400 and all essential oils (ginger oil, eucalyptus oil, basil oil, and clove oil) were added together to give mixture D.
2. Mixtures A and B were mixed together, and then mixture C was added to form a blend.
3. Mixture D was added to this blend, and then glycerol and benzalkonium chloride were added.
4. The volume was made up to 100 ml with water for injection.
5. This mixture was homogenized at 8000-9000 rpm for 15-20 minutes.
6. The homogenized composition was filtered through a Whatman filter (paper of size 11 μm).
7. The composition was dispensed into spray bottles.

### Example 8—Antimicrobial/Germ Kill Assay

This study was conducted to evaluate the antimicrobial activity of the compositions of examples 7B and 7C. The assay measured the changes in a population of aerobic microorganisms within a specified sampling time (30 or 60 seconds) when Test Item (compositions of examples 7B and 7C) was present.

Test Item (composition of example 7B or 7C) was brought into contact with a known population of microorganisms for a specified period of time (30 or 60 seconds) at room temperature. Then the sample was neutralised to quench the antimicrobial activity of the Test Item, and the surviving microorganisms were enumerated. The percent reduction was calculated by comparison with the microbial population before treatment.

TABLE 6
composition of example 7B

| | % reduction | % reduction |
|---|---|---|
| Microorganisms | after 30 sec | after 60 sec |
| Salmonella abony | 99.998 | 99.999 |
| Staphylococcus aureus | 99.874 | 99.895 |
| Escherichia coli | 99.912 | 99.942 |
| Aspergillus brasiliensis | 99.954 | 99.980 |
| Candida albicans | 99.979 | 99.984 |
| Listeria monocytogenes | 99.852 | 99.891 |
| Staphylococcus epidermidis | 99.953 | 99.967 |

TABLE 7
composition of example 7C

| | % reduction | % reduction |
|---|---|---|
| Microorganisms | after 30 sec | after 60 sec |
| Salmonella abony | 99.992 | 99.999 |
| Staphylococcus aureus | 99.998 | 99.998 |
| Escherichia coli | 99.998 | 99.999 |
| Aspergillus brasiliensis | 93.239 | 94.376 |
| Candida albicans | 99.999 | 99.999 |
| Listeria monocytogenes | 99.999 | 99.999 |
| Staphylococcus epidermidis | 99.998 | 99.999 |

The study results are summarised in Tables 6 and 7. Compositions of examples 7B and 7C both showed substantial antimicrobial activity against bacteria (such as Salmonella abony, Staphylococcus aureus, Escherichia coli, Listeria monocytogenes, and Staphylococcus epidermidis) as well as and fungi (such as Aspergillus brasiliensis and Candida albicans).

### Example 9—Virucidal Effect Against SARS-CoV-2 and Influenza (H1N1)

Study A

This study was conducted to evaluate the virucidal activity of the composition of example 7A against SARS-COV-2 and Influenza A (H1N1) virus in vitro. Test Item (composition of example 7A) was tested for virucidal activity by liquid-liquid contact of virus solution with Test Item. Test Item at two test concentrations was incubated with the causative agent of novel coronavirus (SARS-CoV-2 strain USA-WA1/2020) and influenza A (H1N1 pdmo9) for 5 minutes. Subsequently, virus incubated with Test Item was neutralized and added to a confluent layer of host cells. Surviving virus was quantified by standard end-point dilution assay. The Reed-Muench method was used to determine end-point titers (50% cell culture infectious dose, CCID50) of the samples, and the log reduction value (LRV) of the Test Item compared to the negative (water) control was calculated (LRV<1 indicates no virucidal activity, LRV>1 indicates virucidal activity).

Study Design A

Host Cells—SARS-COV-2: VeroE6, Influenza: MDCK

Pre-Incubation of Test Item with virus—5 minutes

Incubation of cells post infection with virus—5 days
SARS-CoV-2 virus stock was prepared by growing virus in VeroE6 cells. Influenza A (H1N1) virus stock was prepared by growing virus in MDCK cells. Test medium used was MEM supplemented with 10 U/mL trypsin, 1 μg/mL EDTA, and 50 μg/mL gentamicin.
Test Item was mixed with virus solution at two concentrations (called 90% and 50%) and incubated together at room temperature for 5 minutes. 90% —Test Item was mixed with virus solution, so that there was 90% composition of example 7A and 10% virus by volume. 50% —Test Item was mixed with virus solution, so that there was 50% composition of example 7A and 50% virus by volume.
Following the contact period, solutions were neutralized by 1/10 dilution in test medium.
Surviving virus was quantified by standard end-point dilution assay.
Samples were serially diluted using eight 10-fold dilutions in test medium and added to host cells.
Plates were incubated at 37° C. with 5% CO₂.
On day 5 post-infection, plates were scored for the presence or absence of viral cytopathic effect (CPE). The Reed-Muench method was used to determine end-point titers (50% cell culture infectious dose, CCID50) of the samples, and the log reduction value (LRV) of the Test Item.

Results A

The study results are summarised in Tables 8 and 9.

TABLE 8

| Concentration (% v/v) of | | | |
|---|---|---|---|
| Test Item incubated with | Final essential | | % Virucidal |
| SARS-CoV-2 virus | oil concentration | LRV | effect |
| 90% | 1.8% | >1.8 log | 90% |
| 50% | 1% | >1.8 log | 90% |

TABLE 9

| Concentration (% v/v) of | | | | |
|---|---|---|---|---|
| Test Item incubated with | Final essential | | | % Virucidal |
| influenza A virus | oil concentration | LRV | | effect |
| 90% | 1.8% | >3.8 | log | 99.9% |
| 50% | 1% | 1.8 | log | 90% |

The composition of example 7A demonstrated virucidal activity against SARS-CoV-2 and influenza A (H1N1) when tested at two different concentrations for 5 minutes.

Study B

This study was conducted to evaluate the virucidal activity of the buffered compositions of examples 7E to 7G and the non-buffered compositions of examples 7J to 7M, all of which comprise only one essential oil each, against SARS-COV-2 in vitro.

Study Design B

Host cells—SARS-COV-2: Vero76

Pre-Incubation of Test Item with virus—5 minutes

Incubation of cells post infection with virus—7 days
SARS-CoV-2 virus stock was prepared by growing virus in Vero76 cells.
Test medium used was DMEM supplemented with penicillin (100 units/mL), streptomycin (100 μg/mL), amphotericin B (0.25 μg/mL) and 2% FBS.
Test Item was mixed with virus solution, so that there was 90% composition of one of examples 7E to 7G or 7J to 7M and 10% virus by volume, and incubated at room temperature for 5 minutes.
Following the contact period, solutions were neutralized by 1/10 dilution in test medium.
Surviving virus was quantified by standard end-point dilution assay.
Samples were serially diluted using six 10-fold dilutions in test medium and added to host cells.
Plates were incubated at 37° C. with 5% CO₂.
On day 7 post-infection, plates were scored for the presence or absence of viral cytopathic effect (CPE). The Reed-Muench method was used to determine end-point titers (50% cell culture infectious dose, CCID50) of the samples, and the log reduction value (LRV) of the Test Item.

Results B

The study results are summarised in Table 16.

TABLE 16

| Composition | LRV | % Virucidal effect |
|---|---|---|
| 7E | 2.27 log | >99% |
| 7F | 1.27 log | >94% |
| 7G | 2.27 log | >99% |
| 7J | 0.94 log | >88% |
| 7K | 1.10 log | >92% |
| 7L | 0.27 log | >46% |
| 7M | 2.27 log | >99% |

Clove oil, eucalyptus oil, ginger oil and basil oil all showed virucidal activity against SARS-CoV-2 when tested for 5 minutes in non-buffered composition. Moreover, virucidal activity was improved when the oils were used in a buffered composition.

### Example 10—Protease Inhibition In Vitro

In this study, the effect of the composition of example 7B was studied on inhibition of proteases such as cathepsin L, 3CL, furin and DPP4, which are relevant in COVID-19, using cell-free assays. Cathepsin L mediates the cleavage of the S1 subunit of the coronavirus surface spike glycoprotein and thus facilitates coronavirus entry into human host cells, virus and host cell endosome membrane fusion, and viral RNA release for next round of replication. 3C-like protease (3CLpro) is essential for SARS-CoV replication. DPP4 interacts with spike glycoprotein Sib domain to promote virus entry. Furin plays a role in the cleavage of SARS-CoV-2 and its entry into the host cell. Purified proteases were incubated with Test Item (composition of example 7B)/Positive Control and corresponding fluorogenic substrates were used to evaluate the inhibitory effect of Test Item/Positive Control.

Study Design

Procedure
Protease inhibition assays were performed using cell-free biochemical kits from BPS Bioscience, US, as per manufacturer's protocol.
Positive controls (PC) were provided in the kits.
The composition of example 7B was diluted with assay buffer from the kit to obtain the desired final % concentrations of essential oils as indicated in tables 10-13.
Percentage inhibition in fluorescence values was determined in comparison with enzyme control (i.e. without Test Item/Positive Control).

TABLE 10
3CL protease

| Sample | Concentration | Percentage inhibition |
|---|---|---|
| GC376 (μM) (PC) | 0.1 | 24.2 ** |
| | 1 | 65.4 ** |
| | 10 | 82.4 ** |
| | 100 | 79.1 ** |
| Composition of example 7B | 0.001 | −16.6 |
| (essential oil %) | 0.005 | −1.9 |
| | 0.01 | 0.0 |
| | 0.1 | −1.9 |
| | 0.2 | 16.0 ** |

(** represent significant values, where p < 0.001, as compared to control)

TABLE 11
DPP4 protease

| Sample | Concentration | Percentage inhibition |
|---|---|---|
| Sitagliptin (μM) (PC) | 0.001 | 32.4 ** |
| | 0.01 | 76.2 ** |
| | 0.1 | 87.1 ** |
| | 1 | 99.5 ** |
| Composition of example 7B | 0.001 | 18.4 * |
| (essential oil %) | 0.005 | 16.1 * |
| | 0.01 | 15.4 * |
| | 0.025 | 14.4 * |
| | 0.05 | 9.4 |

(* and ** represent significant values, where p < 0.01 and p < 0.001 respectively, as compared to control)

TABLE 12
cathepsin L protease

| Sample | Concentration | Percentage inhibition |
|---|---|---|
| E-64 (μM) (PC) | 0.0001 | −15.5 |
| | 0.001 | −1.1 |
| | 0.01 | 17.2 ** |
| | 0.1 | 56.3 ** |
| Composition of example 7B | 0.001 | −93.1 |
| (essential oil %) | 0.01 | 43.7 ** |
| | 0.025 | 66.7 ** |
| | 0.05 | 48.9 ** |
| | 0.1 | 31.0 ** |

(** represent significant values, where p < 0.001, as compared to control)

TABLE 13
furin protease

| Sample | Concentration | Percentage inhibition |
|---|---|---|
| Chloromethylketone (μM) (PC) | 0.001 | 45.6 ** |
| | 0.01 | 95.3 ** |
| | 0.05 | 100.6 ** |
| Composition of example 7B | 0.001 | −13.4 |
| (essential oil %) | 0.01 | −3.2 |
| | 0.025 | 10.3 ** |
| | 0.05 | 26.2 ** |
| | 0.1 | 45.7 ** |

(** represent significant values, where p < 0.001, as 3compared to control)

Results

The study results are summarised in Tables 10-13. The composition of example 7B resulted in 16%, 18.4%, 66.7% and 45.7% inhibition of 3CL, DPP4, cathepsin L and furin protease respectively. The results demonstrated that the composition of example 7B led to significant inhibition (p<0.01 or p<0.001) of 3CL, DPP4, cathepsin L and furin protease as compared to enzyme control.

### Example 11—Inhibition of Spike S1-ACE2 Interaction

SARS-CoV2 enters the human body via SpikeS1 protein that binds to ACE2 receptors present on cells of the nasal mucosa and lungs. Inhibition of binding of SpikeS1 protein and ACE2 receptors has been widely considered as a preventive strategy for COVID-19. In this study, the effect of the composition of example 7B was studied on binding inhibition of SpikeS1 protein with ACE2 receptors, using an ELISA-like colorimetric kit in a cell-free assay. Different concentrations of Test Item (composition of example 7B) and Positive Control along with ACE2 Inhibitor Screening reagent were added in a 96-well plate (pre-coated with Rabbit Fc-tagged SARS-Cov-2 Spike S1 RBD) and incubated. Further, Spike Inhibitor Screening reagent was added to the plate and incubated. Finally, the plate was treated with Anti-His-HRP Conjugate, followed by addition of TMB Substrate to produce absorbance, which was then measured using a spectrophotometer at 450 nm wavelength.

Study Design

Procedure
The inhibition of SpikeS1-ACE2 interaction was studied using a cell-free assay kit, SARS-CoV-2 Spike-ACE2 Interaction Inhibitor Screening Assay kit from Cayman Chemical Company, US, as per manufacturer's protocol.
Emodin was used as Positive Control (PC).
The composition of example 7B was diluted with serum free medium to obtain the desired final % concentrations of essential oils as indicated in Table 14.
SARS-CoV2 Inhibitor Control was used as internal kit control (provided in the kit).
Percentage inhibition in SpikeS1-ACE2 interaction was determined as compared to 100% initial activity.

TABLE 14

| | | Percentage inhibition |
|---|---|---|
| Sample | Concentration | (wrt 100% initial activity) |
| 100% Initial Activity | | 0.0 |
| SARS-CoV2 Inhibitor | | 81.9 ** |
| Emodin (PC) (μg/ml) | 0.01 | 35.5 ** |
| | 0.1 | 38.9 ** |
| | 1 | 44.3 ** |
| | 10 | 48.9 ** |
| | 50 | 62.8 ** |
| Composition of example 7B | 0.001 | 48.5 ** |
| (essential oil %) | 0.005 | 52.1 ** |
| | 0.01 | 42.8 ** |
| | 0.05 | 47.5 ** |
| | 0.1 | 55.2 ** |
| | 0.33 | 63.9 ** |

** represent significant values, where p < 0.001, as compared to control

Results

The composition of example 7B significantly (p<0.001) inhibited SpikeS1-ACE2 binding by 63.9% as compared to control. Based on these results, it can be concluded that the composition of example 7B inhibits binding of SpikeS1 protein and ACE2 receptors.

### Example 12

This example was conducted to show that BES (as used in example 4) can be replaced by taurine in the buffer compositions of the present invention.

Aqueous buffer solutions A to F were prepared by stirring the components set out in Table 15 in 1 L of ultrapure water (ATSM Type I, 18.2 MΩ/cm at 25° C.).

TABLE 15

| | | Buffer A | Buffer B | Buffer C | Buffer D | Buffer E | Buffer F |
|---|---|---|---|---|---|---|---|
| | Molecular | Molar | Molar | Molar | Molar | Molar | Molar |
| Component | weight | Amount | Amount | Amount | Amount | Amount | Amount |
| BES | 213.25 | 5 mmoles | 10 mmoles | 20 mmoles | — | — | — |
| Taurine | 125.14 | — | — | — | 5 mmoles | 10 mmoles | 20 mmoles |
| NaHCO₃ | 84.01 | 25 mmoles | 25 mmoles | 25 mmoles | 25 mmoles | 25 mmoles | 25 mmoles |
| pH at 37° C. | | 7.18 | 6.99 | 6.81 | 7.70 | 7.57 | 7.43 |

It was found that all six buffer solutions A to F are buffer compositions having a suitable pH for use in the treatments of the present invention.

### Example 13—Manufacturing Example

Thiamine pyrophosphate chloride was prepared as a 0.4 mg/mL stock solution in MilliQ endotoxin-free purified water and stored frozen in dark glass vials. Choline chloride was prepared as a 17.45 mg/mL stock solution in MilliQ endotoxin-free purified water and stored frozen in glass vials. Human recombinant insulin was prepared as a 0.5 mIU/mL stock solution in MilliQ endotoxin-free purified water acidified to pH 2.4 with 0.1N hydrochloric acid and stored frozen in glass vials.

In the following preparations, MilliQ endotoxin-free purified water was used throughout, both in the initial stirring, and in the final dilution.

For the preparation, a stainless steel container was filled with 8 litres of MilliQ endotoxin-free purified water and the following components were added while constantly stirring, in the following order: 642.96 grams of sodium chloride, 37.28 grams of potassium chloride, 18.38 grams of calcium chloride dihydrate, 9.14 grams of magnesium chloride hexahydrate, 1.363 grams zinc chloride, 106.61 grams of N,N-bis(2-hydroxyethyl)-2-aminoethanesulphonic acid (BES), optionally 1.84 milligrams of thiamine pyrophosphate chloride (using 4.6 mL of the stock solution), 0.9899 grams of L-carnitine, 0.1396 grams of choline chloride (using 8 mL of the stock solution), 1.013 grams of glycerol, optionally 2.8 mIU of human recombinant insulin (using 5.6 mL of the stock solution), 0.310 grams of L-aspartate sodium salt, 180.2 grams of anhydrous D-glucose, 5.07 grams of L-glutamate sodium salt and 5.84 grams of L-glutamine. The mixture was stirred until completely dissolved and then the final volume of 10 litres was produced by adding further MilliQ endotoxin-free purified water. The solution was filtered through a sterile filter (0.2 μm Sartobran PH) into 100 mL sterile sealed glass bottles. This solution is a lox concentrate of the solution intended for use. The concentrate can be stored under dark conditions at 3-8° C. for up to five years.

For use, 100 mL of the concentrate was diluted with 900 mL of double deionised or MilliQ endotoxin-free purified water to 1 litre with the addition of 2.1 grams of endotoxin-free sodium bicarbonate and stored at 8-10° C. prior to use. For storage stability, it is preferable not to add sodium bicarbonate to the concentrate before it is stored.

### Example 14—Ex-Vivo Permeation Study

Eugenol is an active component of clove oil and basil oil. An ex-vivo permeation study was conducted to ascertain whether eugenol permeates through goat nasal mucosa.

Goat nasal mucosa was kept in phosphate buffer pH 6.4 and then cartilage was removed carefully. The nasal mucosa was stabilized in a Franz diffusion cell (Logan Instrument Corporation, NJ USA) containing phosphate buffer pH 6.4 in both donor and accepter compartments. The composition of example 7B, 7D, 7N or 70 (3 ml) was dispensed in the donor compartment. Eugenol permeated through an area of 0.15 cm². ml of sample was removed from the acceptor compartment from the time interval of 5 min to 8 hour and an equal volume of PBS pH 6.4 was added to maintain sink conditions. Samples were analysed by GC-MS. The amount of eugenol which had permeated through the nasal mucosa was calculated using the following formula:

$$amount of eugenol permeated = \frac{concentration ( mg / L ) \times volume of release medium ( mL )}{permeation area ( {cm}^{2} )}$$

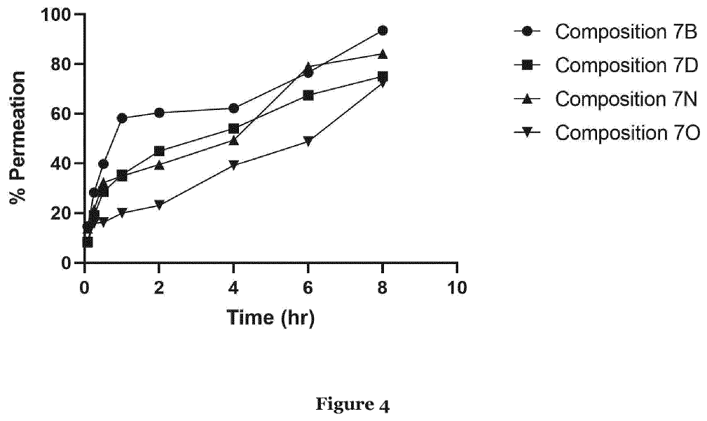

A graph of the amount of eugenol permeated vs time is shown in FIG. 4. The slope gives the value of steady state flux (J).

RESULTS: The ex-vivo permeation of eugenol through goat nasal mucosa in 8 hours was found to be 94% for composition 7B, 84% for composition 7N (without buffer), 75% for composition 7D (without zinc ions), and 72% for composition 70 (without buffer and without zinc ions). This shows that both the buffer and the zinc ions increase eugenol permeation.

It will be understood that the present invention has been described above by way of example only. The examples are not intended to limit the scope of the invention. Various modifications and embodiments can be made without departing from the scope and spirit of the invention, which is defined by the following claims only.

```
SEQ ID NO: 1 is the following sequence, wherein Xaa can be any naturally
occurring amino acid:
Ser Leu Thr His Arg Lys Phe Gly Gly Ser Gly Gly Ser Pro Phe
1 5 10 15
Ser Gly Leu Ser Ser Ile Ala Val Arg Ser Gly Ser Tyr Leu Asp
                                20 25 30
Xaa Ile Ile Ile Asp Gly Val His His Gly Gly Ser Gly Gly Asn
                                35 40 45
Leu Ser Pro Thr Phe Thr Phe Gly Ser Gly Glu Tyr Ile Ser Asn
                                50 55 60
Met Thr Ile Arg Ser Gly Asp Tyr Ile Asp Asn Ile Ser Phe Glu
                                65 70 75
Thr Asn Met Gly Arg Arg Phe Gly Pro Tyr Gly Gly Ser Gly Gly
                                80 85 90
Ser Ala Asn Thr Leu Ser Asn Val Lys Val Ile Gln Ile Asn Gly
                                95 100 105
Ser Ala Gly Asp Tyr Leu Asp Ser Leu Asp Ile Tyr Tyr Glu Gln
                                110 115 120
Tyr
```

1. A composition comprising one or more of clove oil, eucalyptus oil, basil oil, ginger oil, an extract or component of any of these, or a combination thereof, for use in the treatment, prophylactic treatment or amelioration of an airborne viral infection, or for use in reducing or preventing viral replication in a subject infected with an airborne virus or exposed to an airborne virus capable of causing an airborne viral infection in the subject.

2. The composition of claim 1, further comprising a buffer.

3. An aqueous composition comprising:

| | | |
|---|---|---|
| (i) | from 1 to 500 mmoles/L | buffer; and |
| (ii) | from 0.1 to 200 g/L | essential oil, or an extract or component thereof, |
| | | with antiviral, antibacterial or antifungal activity. |

4. The composition of claim 3, wherein the essential oil is selected from clove oil, eucalyptus oil, basil oil, ginger oil, saffron oil, cannabis oil, tea tree oil, black seed oil, an extract or component of any of these, or a combination thereof.

5. The composition of any one of the preceding claims, wherein the extract or component of the essential oil is selected from eugenol, zingiberene, eucalyptol, jensenone, ursolic acid, caryophyllene, estragole, 1,8-cineole, camphor, thymol, eugenol epoxide, apigenin, linalool, rosmarinic acid, 6-gingerol, 6-shogaol, gingeronone A, crocetin, crocin, picrocrocin, safranal, caflanone, tetrahydrocannabinol, cannabidiol (or its metabolite 7-hydroxycannabidiol), cannabinol, tetrahydrocannabivarin, terpinen-4-ol, terpinolene, α-terpineol, thymoquinone, ar-curcumene, β-sesquiphellandrene, α-farnesene, α-phellandrene, β-phellandrene, camphene, β-bisabolene, α-terpinene, γ-terpinene, p-cymene, α-pinene, or a mono- or polyunsaturated fatty acid such as palmitoleic acid, heptadecenoic acid, oleic acid, linoleic acid, α-linolenic acid, γ-linolenic acid, arachidonic acid, or erucic acid.

6. The composition of any one of the preceding claims, wherein the essential oil, or the extract or component thereof, has antiviral activity.

7. The composition of any one of claims 2 to 6, wherein the buffer comprises hydrogen carbonate ions or an equivalent thereof.

8. The composition of any one of claims 2 to 7, wherein the buffer comprises, consists of or consists essentially of (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; and (ii) hydrogen carbonate ions or an equivalent thereof.

9. The composition of any one of claims 2 to 8, wherein the buffer comprises, consists of or consists essentially of:
(i) from 1 to 100 mmoles/L Good's buffer, aminosulphonic acid, aminosulphinic acid, phosphate, phosphite, heteroaryl, phenolic acid, amino acid, peptide, peptide equivalent, polymeric buffer, urea, ionic liquid buffer, or a combination thereof; and
(ii) from 21 to 35 mmoles/L hydrogen carbonate ions or an equivalent thereof.

10. The composition of any one of claims 2 to 9, wherein the buffer comprises, consists of or consists essentially of (i) one or more phosphates, and (ii) hydrogen carbonate ions.

11. The composition of any one of claims 2 to 10, wherein the buffer comprises, consists of or consists essentially of KH₂PO₄, Na₂HPO₄ and NaHCO₃.

12. The composition of any one of the preceding claims, further comprising from 0.1 to 200 μmoles/L zinc ions.

13. The composition of any one of the preceding claims, further comprising from 1 to 100 μmoles/L transferrin and/or iron ions.

14. The composition of any one of the preceding claims, comprising:

| | | |
|---|---|---|
| (i) | from 1 to 500 mmoles/L | buffer; |
| (ii) | from 0.1 to 50 g/L | clove oil or an extract or component thereof; |
| (iii) | from 0.1 to 50 g/L | eucalyptus oil or an extract or component thereof; |
| (iv) | from 0.1 to 50 g/L | basil oil or an extract or component thereof; and |
| (v) | from 0.1 to 50 g/L | ginger oil or an extract or component thereof. |

15. The composition of any one of the preceding claims, comprising:

| | | |
|---|---|---|
| (i) | from 1 to 500 mmoles/L | buffer; |
| (ii) | from 0.1 to 200 μmoles/L | zinc ions; |
| (iii) | from 0.1 to 50 g/L | clove oil or an extract or component thereof; |
| (iv) | from 0.1 to 50 g/L | eucalyptus oil or an extract or component thereof; |
| (v) | from 0.1 to 50 g/L | basil oil or an extract or component thereof; and |
| (vi) | from 0.1 to 50 g/L | ginger oil or an extract or component thereof. |

16. The composition of any one of the preceding claims, comprising:

| | | |
|---|---|---|
| (i) | from 25 to 250 mmoles/L | one or more phosphates; |
| (ii) | from 1 to 45 mmoles/L | hydrogen carbonate ions; |
| (iii) | from 0.1 to 10 g/L | clove oil or an extract or component thereof; |
| (iv) | from 0.1 to 10 g/L | eucalyptus oil or an extract or component thereof; |
| (v) | from 0.1 to 10 g/L | basil oil or an extract or component thereof; and |
| (vi) | from 0.1 to 10 g/L | ginger oil or an extract or component thereof. |

17. The composition of any one of the preceding claims, comprising:

| | | |
|---|---|---|
| (i) | from 25 to 250 mmoles/L | one or more phosphates; |
| (ii) | from 1 to 45 mmoles/L | hydrogen carbonate ions; |
| (iii) | from 0.1 to 50 μmoles/L | zinc ions; |
| (iv) | from 0.1 to 10 g/L | clove oil or an extract or component thereof; |
| (v) | from 0.1 to 10 g/L | eucalyptus oil or an extract or component thereof; |
| (vi) | from 0.1 to 10 g/L | basil oil or an extract or component thereof; and |
| (vii) | from 0.1 to 10 g/L | ginger oil or an extract or component thereof. |

18. The composition of any one of the preceding claims, wherein the composition further comprises:

| (i) | calcium ions and magnesium ions at a molar concentration ratio of from 5:1 to | |
|---|---|---|
| | 1:1, wherein said calcium ions are at a concentration of from 0.1 to 2.5 | |
| | mmoles/L; | |
| (ii) | from 2.5 to 20 mmoles/L | potassium ions; |
| (iii) | from 96 to 126 mmoles/L | chloride ions; and |
| (iv) | from 100 to 300 mmoles/L | sodium ions. |

19. The composition of any one of the preceding claims, wherein the composition further comprises one or more additional components selected from NaCl, KCl, MgCl₂, xylitol, EDTA, CaCl₂, glycerol, HPMC, PEG 400, poloxamer 188, benzalkonium chloride and sodium hyaluronate.

20. The composition of any one of the preceding claims, wherein the composition is an emulsion or comprises microdroplets.

21. A buffer composition having a pH of from 6.7 to 7.9 at a temperature of 37° C., for use in the treatment, prophylactic treatment or amelioration of an airborne viral infection, or for use in reducing or preventing viral replication in a subject infected with an airborne virus or exposed to an airborne virus capable of causing an airborne viral infection in the subject.

22. An aqueous composition comprising (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; and (ii) hydrogen carbonate ions or an equivalent thereof; wherein the aqueous composition is for use in the treatment, prophylactic treatment or amelioration of an airborne viral infection, or for use in reducing or preventing viral replication in a subject infected with an airborne virus or exposed to an airborne virus capable of causing an airborne viral infection in the subject.

23. The composition of any one of the preceding claims, wherein the composition is an aqueous composition comprising:

| (i) | from 1 to 100 mmoles/L (preferably from 1 to 12 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | calcium ions and magnesium ions at a molar concentration ratio of from 5:1 to | |
| | 1:1, wherein said calcium ions are at a concentration of from 0.1 to 2.5 | |
| | mmoles/L; | |
| (iii) | from 21 to 35 mmoles/L | hydrogen carbonate ions or an equivalent thereof; |
| (iv) | from 2.5 to 6.2 mmoles/L | potassium ions; |
| (v) | from 96 to 126 mmoles/L | chloride ions; and |
| (vi) | from 100 to 150 mmoles/L | sodium ions. |

24. An aqueous composition comprising (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; (ii) hydrogen carbonate ions or an equivalent thereof; and (iii) zinc ions.

25. An aqueous composition comprising (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; (ii) hydrogen carbonate ions or an equivalent thereof; and (iii) transferrin and/or iron ions.

26. The composition of any one of the preceding claims, wherein the composition is an aqueous composition comprising:

| (i) | from 1 to 100 mmoles/L (preferably from 1 to 12 mmoles/L) Good's buffer, | |
|---|---|---|
| | aminosulphonic acid, aminosulphinic acid, phosphate, phosphite, heteroaryl, | |
| | phenolic acid, amino acid, peptide, peptide equivalent, polymeric buffer, urea, | |
| | ionic liquid buffer, or a combination thereof; | |
| (ii) | calcium ions and magnesium ions at a molar concentration ratio of from 5:1 to | |
| | 1:1, wherein said calcium ions are at a concentration of from 0.1 to 2.5 | |
| | mmoles/L; | |
| (iii) | from 21 to 35 mmoles/L | hydrogen carbonate ions or an equivalent thereof; |
| (iv) | from 2.5 to 6.2 mmoles/L | potassium ions; |
| (v) | from 96 to 126 mmoles/L | chloride ions; |
| (vi) | from 100 to 150 mmoles/L | sodium ions; and |
| (vii) | from 0.1 to 200 μmoles/L | zinc ions. |

27. The composition of any one of the preceding claims, wherein the composition is an aqueous composition comprising:
(i) from 1 to 100 mmoles/L (preferably from 1 to 12 mmoles/L) Good's buffer, aminosulphonic acid, aminosulphinic acid, phosphate, phosphite, heteroaryl, phenolic acid, amino acid, peptide, peptide equivalent, polymeric buffer, urea, ionic liquid buffer, or a combination thereof;

| (ii) | calcium ions and magnesium ions at a molar concentration ratio of from 5:1 to | |
|---|---|---|
| | 1:1, wherein said calcium ions are at a concentration of from 0.1 to 2.5 | |
| | mmoles/L; | |
| (iii) | from 21 to 35 mmoles/L | hydrogen carbonate ions or an equivalent thereof; |
| (iv) | from 2.5 to 6.2 mmoles/L | potassium ions; |
| (v) | from 96 to 126 mmoles/L | chloride ions; |
| (vi) | from 100 to 150 mmoles/L | sodium ions; |
| (vii) | from 1 to 100 μmoles/L | transferrin; and |
| (viii) | from 1 to 100 μmoles/L | iron ions. |

28. The composition of any one of the preceding claims, further comprising one or more of:

| | | |
|---|---|---|
| (a) | from 2 to 11 mmoles/L | glucose; |
| (b) | from 50 to 150 μmoles/L | glycerol; |
| (c) | from 7 to 15 μmoles/L | choline ions; |
| (d) | from 5 to 400 μmoles/L | glutamate; |
| (e) | from 5 to 200 μmoles/L | aspartate; |
| (f) | from 100 to 2000 μmoles/L | glutamine; |
| (g) | from 20 to 215 μmoles/L | pyroglutamate; |
| (h) | from 20 to 200 μmoles/L | arginine; |
| (i) | from 1 to 250 μmoles/L | thiamine pyrophosphate ions; |
| (j) | from 40 to 100 μmoles/L | carnitine; |
| (k) | from 5 to 600 mIU/L | porcine or human insulin; |
| (l) | from 20 to 200 μmoles/L | hyaluronic acid; |
| (m) | from 1 to 100 μmoles/L | transferrin; |
| (n) | from 20 to 250 μmoles/L | leucine; |
| (o) | from 10 to 100 μmoles/L | linoleic acid; |
| (p) | from 200 to 1000 μmoles/L | cholesterol; |
| (q) | from 20 to 500 μmoles/L | pyridoxal-5-phosphate; or |
| (r) | from 10 to 250 μmoles/L | chitosan. |

29. The composition of any one of the preceding claims, further comprising an antibiotic, an emulsifying agent and/or an antioxidant.

30. The composition of any one of the preceding claims, having a pH of from 6.7 to 7.9 at a temperature of 37° C.

31. The composition of any one of the preceding claims, wherein the composition is suitable for inhalation into the oral cavity, upper respiratory tract, lower respiratory tract, nasal cavity, pharynx, larynx, trachea, bronchi or lungs.

32. The composition of any one of the preceding claims, wherein the composition is suitable for administration by inhaler, nebulizer, nasal spray, mouth spray, bronchial spray, nasal drops, nasal wash, nasal lavage, neti pot, nasal packing, mouthwash or gargle, or in the form of a cream, gel, emulsion or ointment.

33. The composition of any one of the preceding claims, wherein the composition is suitable for application to a mask or other face covering, or wherein the composition is suitable for use in a receptacle configured to allow an oxygen-containing gas to be bubbled through the composition prior to inhalation of the gas by a subject, or wherein the composition is suitable for diffusing or spraying into the air prior to inhalation of the air by a subject.

34. The composition of any one of the preceding claims, for use in the treatment, prophylactic treatment or amelioration of an airborne viral infection, or for use in reducing or preventing viral replication in a subject infected with an airborne virus or exposed to an airborne virus capable of causing an airborne viral infection in the subject.

35. The composition of claim 34, wherein the airborne viral infection is caused by (i) an RNA virus, such as a coronavirus (including a coronavirus selected from MERS-CoV, SARS-CoV, and SARS-CoV-2), an influenza virus (including an influenza virus selected from influenza A virus, influenza B virus, influenza C virus, and parainfluenza virus), a rhinovirus, Measles virus, Mumps virus, Rubella virus, or human respiratory syncytial virus, or (ii) a DNA virus, such as Parvovirus B19, an adenovirus, an adeno-associated virus, a herpes virus (including a herpes virus selected from Varicella-Zoster virus (VZV or HHV-3), Epstein-Barr virus (EBV or HHV-4), human herpes virus 6 (HHV-6A and HHV-6B), and human herpes virus 7 (HHV-7)), a polyomavirus (including a polyomavirus selected from BK polyomavirus and WU polyomavirus), or Variola virus.

36. A method of preparing the composition of any one of claims 1 to 35, comprising combining all components with a solvent (preferably water), optionally making up to the desired volume, optionally filtering and optionally storing in a sealed vessel.

37. A concentrate for the preparation of the composition of any one of claims 1 to 35, comprising water and all components, wherein the concentrate is dilutable with water to form the composition of any one of claims 1 to 35.

38. A concentrate for the preparation of the composition of any one of claims 1 to 35, comprising water and all components except the hydrogen carbonate ions or the equivalent thereof and their countercations, wherein the concentrate is dilutable with water comprising the hydrogen carbonate ions or the equivalent thereof and their countercations to form the composition of any one of claims 1 to 35.

39. A method of treatment, prophylactic treatment or amelioration of an airborne viral infection in a subject, the method comprising administering an effective amount of the composition of any one of claims 1 to 35 to the subject.

40. A method of reducing or preventing viral replication in a subject infected with an airborne virus or exposed to an airborne virus capable of causing an airborne viral infection in the subject, the method comprising administering an effective amount of the composition of any one of claims 1 to 35 to the subject.

41. The method of claim 39 or 40, wherein the airborne viral infection is caused by (i) an RNA virus, such as a coronavirus (including a coronavirus selected from MERS-CoV, SARS-CoV, and SARS-CoV-2), an influenza virus (including an influenza virus selected from influenza A virus, influenza B virus, influenza C virus, and parainfluenza virus), a rhinovirus, Measles virus, Mumps virus, Rubella virus, or human respiratory syncytial virus, or (ii) a DNA virus, such as Parvovirus B19, an adenovirus, an adeno-associated virus, a herpes virus (including a herpes virus selected from Varicella-Zoster virus (VZV or HHV-3), Epstein-Barr virus (EBV or HHV-4), human herpes virus 6 (HHV-6A and HHV-6B), and human herpes virus 7 (HHV-7)), a polyomavirus (including a polyomavirus selected from BK polyomavirus and WU polyomavirus), or Variola virus.

42. A method of reducing the risk of viral infection with an airborne virus in a subject, the method comprising applying the composition of any one of claims 1 to 35 to a mask or other face covering.

43. A method of reducing the risk of viral infection with an airborne virus in a subject, the method comprising bubbling an oxygen-containing gas through the composition of any one of claims 1 to 35 prior to inhalation of the gas by the subject.

44. A method of reducing the risk of viral infection with an airborne virus in a subject, the method comprising diffusing or spraying the composition of any one of claims 1 to 35 into the air prior to inhalation of the air by the subject.

45. A spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising an aqueous composition comprising (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; and (ii) hydrogen carbonate ions or an equivalent thereof.

46. A spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising the composition of any one of claims 1 to 35.

47. A spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising (i) the concentrate of claim 37, and (ii) water.

48. A spray, inhaler, nebulizer, nasal spray, mouth spray, bronchial spray, nasal drops, nasal wash, nasal lavage, neti pot, nasal packing, mouthwash or gargle, receptacle, cream, gel, emulsion or ointment, comprising (i) the concentrate of claim 38, and (ii) water comprising hydrogen carbonate ions or an equivalent thereof and their countercations.

49. A mask or other face covering coated or impregnated with an aqueous composition comprising (i) a Good's buffer, an aminosulphonic acid, an aminosulphinic acid, a phosphate, a phosphite, a heteroaryl, a phenolic acid, an amino acid, a peptide, a peptide equivalent, a polymeric buffer, a urea, an ionic liquid buffer, or a combination thereof; and (ii) hydrogen carbonate ions or an equivalent thereof.

50. A mask or other face covering coated or impregnated with the composition of any one of claims 1 to 35.

51. A method of cleaning an object, the method comprising applying the composition of any one of claims 1 to 35 to the object.


SEQUENCE LISTING (1 sequence)

SEQ ID NO: 1 - protein, 121 aa, Artificial Sequence
  Synthetic
  misc_feature (31)..(31): Xaa can be any naturally occurring amino acid
SLTHRKFGGSGGSPFSGLSSIAVRSGSYLDXIIIDGVHHGGSGGNLSPTFTFGSGEYISNMTIRSGDYIDNISFETNMGRRFGPYGGSGGSANTLSNVKVIQINGSAGDYLDSLDIYYEQY